SEQUENCE LISTING (133 sequences)

SEQ ID NO: 1 - RNA, 84 nt, Artificial Sequence
  Synthetic oligonucleotide
  misc_feature (25)...(26): squaramide linkage
cgccccgaggagcgcugcaacagggccgccggcccugccaggcucggggauggcgccccaucguuccaacggcgcucagcgaaa

SEQ ID NO: 2 - RNA, 73 nt, Artificial Sequence
  Synthetic oligonucleotide
ccugccgaggagcgcugcgacccgaaaucacgccgggccaggcucggcaaccgaacaacggcgcucgcaaacc

SEQ ID NO: 3 - RNA, 67 nt, Artificial Sequence
  Synthetic oligonucleotide
uuuccgaggagcguugcgacgggcaccggcccgccaggcucggaaaucuucaccaggcgguggcccg

SEQ ID NO: 4 - RNA, 68 nt, Artificial Sequence
  Synthetic oligonucleotide
guuuccgaggagcguugcgacgggccccgcccgccaggcucggaaauggucaaccgagcggugugucu

SEQ ID NO: 5 - RNA, 73 nt, Artificial Sequence
  Synthetic oligonucleotide
caauccgaggagcgcugcagggccgucgaucgacgguuucaggcucggauuccuuaaacggcgcucauccgac

SEQ ID NO: 6 - RNA, 97 nt, Artificial Sequence
  Synthetic oligonucleotide
ccauccgaggagcguugcagcguuccccagccagccaugagcuggccugcgcggggcgugaggcucggauuucaucccgcaacgacgcucgcccacg

SEQ ID NO: 7 - RNA, 74 nt, Artificial Sequence
  Synthetic oligonucleotide
cacuccgaggagcguugcaacggauggccucgccuuccgccaggcucggaauguuucaacggcgcucgcaguuc

SEQ ID NO: 8 - RNA, 71 nt, Artificial Sequence
  Synthetic oligonucleotide
ucugccgaggagcgcugcgacccuuuaauucgggggccaggcucggcaaugaucaacggcgcucgcaaacc

SEQ ID NO: 9 - RNA, 85 nt, Artificial Sequence
  Synthetic oligonucleotide
cucuccgaggagcguugcagcggcccccacacaucaggcguggucgcgaggcucggagucauaccugcaacgacgcucgcccacg

SEQ ID NO: 10 - RNA, 83 nt, Artificial Sequence
  Synthetic oligonucleotide
ccguucgaggagcguugcgacacacccaugcuuuuuuggggugcuaggcucgaacaagaaauaaacaacggcgcucacgcuuc

SEQ ID NO: 11 - RNA, 82 nt, Artificial Sequence
  Synthetic oligonucleotide
gcgccgaggagcgcugcgacggcccgucgagggccgccaggcucggcggggacaaucgguuuuccaacggcgcucuguuuau

SEQ ID NO: 12 - RNA, 78 nt, Artificial Sequence
  Synthetic oligonucleotide
acuuucgaggagcguugcaacucccgcccgggagccaggcucgaaagucaucccccgaugcaacggcgcucacccgca

SEQ ID NO: 13 - RNA, 74 nt, Artificial Sequence
  Synthetic oligonucleotide
gacuucgaggagcgcugcgaggccggacccggccccaggcucgaagcggaaucacccaacggcgcucaccugua

SEQ ID NO: 14 - RNA, 91 nt, Artificial Sequence
  Synthetic oligonucleotide
cucgccgaggagcgcugcaacaggacacgucuuggauuccugccaggcucggcgauuaucgggaccuuuaaaccaacggcgcucgguucag

SEQ ID NO: 15 - RNA, 78 nt, Artificial Sequence
  Synthetic oligonucleotide
uuguaccgaggagcgcugcaacggucuaucucgugaggcucggacaaaugaacaacccaacaacggcgcucuuucacu

SEQ ID NO: 16 - RNA, 78 nt, Artificial Sequence
  Synthetic oligonucleotide
cagaccgaggagcgcugcaacgggcauugucccgcgaggcucggcaaggaaggaucgcuacaacggcgcucguucauu

SEQ ID NO: 17 - RNA, 92 nt, Artificial Sequence
  Synthetic oligonucleotide
ccguccgaggggcgcugcagcaggucggucgacaccgguuugucaggcucggauggggcguuaaccauacgcaucaacggcgcccauucgca

SEQ ID NO: 18 - RNA, 73 nt, Artificial Sequence
  Synthetic oligonucleotide
cuuccugaggagcguugcaacccacaucacguggaccaggcucaggacuucauugcaaccacgcucacccgca

SEQ ID NO: 19 - RNA, 106 nt, Artificial Sequence
  Synthetic oligonucleotide
ccguucgaggggcgcugcagcaggccuaggccugucaggcucgaacggagcgcgcucucaggccggccucgccggucugucaacgcaccaacggcgcccauucgca

SEQ ID NO: 20 - RNA, 107 nt, Artificial Sequence
  Synthetic oligonucleotide
cuguccgaggggcgcugcagcaggcaugaccugucaggcucggauggggcguuguugguacagggcucccuguauggacacuaaacgcacaacggcgcccauucgca

SEQ ID NO: 21 - RNA, 97 nt, Artificial Sequence
  Synthetic oligonucleotide
cuguccgaggggcgcugcagcaggcuaggccugucaggcucggauggggcguugcucgcucccgcgagcgcuuaacgcacaacggcgcccauucgca

SEQ ID NO: 22 - RNA, 87 nt, Artificial Sequence
  Synthetic oligonucleotide
ccacccgaggggcgcugcagcagguuccagaaccugucaggcucggauggggcguuuccucgaacgcaucaacggcgcccauucgca

SEQ ID NO: 23 - RNA, 110 nt, Artificial Sequence
  Synthetic oligonucleotide
cuguccgaggggcgcugcagcagguuuacccugucaggcucggauggggcguuguuugucagguugaucacagccggcaagccuuaaacgcacaacggcgcccauucgca

SEQ ID NO: 24 - RNA, 84 nt, Artificial Sequence
  Synthetic oligonucleotide
cauuccgaggagcguugcaacgaccggcccaaguucagacggggcggccgccaggcucggaaguucaaacggcgcucgcaguau

SEQ ID NO: 25 - RNA, 75 nt, Artificial Sequence
  Synthetic oligonucleotide
auuccgaggagcguugcaacggaccggcauccgcccggccgccaggcucggaaguucaaacggcgcucgcacuau

SEQ ID NO: 26 - RNA, 71 nt, Artificial Sequence
  Synthetic oligonucleotide
ucuuccaaggagcguugcagucggccacauggccggucaggcuuggaugaccccaacgacgcucaccugau

SEQ ID NO: 27 - RNA, 82 nt, Artificial Sequence
  Synthetic oligonucleotide
cugccgaggggcgcugcgaccggaucccuugaggccggccaggcucggccaggugguacugcaacaacggcgcccggccaga

SEQ ID NO: 28 - RNA, 68 nt, Artificial Sequence
  Synthetic oligonucleotide
caaccugaggagcgcugcaacggggacuucccgccaggcucagguucauguaacggcgcucgcugacu

SEQ ID NO: 29 - RNA, 88 nt, Artificial Sequence
  Synthetic oligonucleotide
cgcuccgaggagcguugcagcgccucccaccggcagggccggcggggcgugaggcucgggcaucaucuggcaacgacgcucauccaca

SEQ ID NO: 30 - RNA, 78 nt, Artificial Sequence
  Synthetic oligonucleotide
cuuuccgaggagcgcugcaacgggcccuuacggcccgccaggcucggaaaggaaaaucuacaacggcgcucacucaca

SEQ ID NO: 31 - RNA, 112 nt, Artificial Sequence
  Synthetic oligonucleotide
ccuuccgaggagcgcugcaacgggccccacguccccucccggcccgccaggcucggaagggcguuguuugugcccuccgcgugccuaacccccucaacggcgcucacucgca

SEQ ID NO: 32 - DNA, 157 bp, Artificial Sequence
  Synthetic oligonucleotide
ccgtccgaggggcgctgcagcaggtttgttgagtagagtgtgagctccgtaactagttacatcgcaagatgtaactgaatgaaatggtgaaggacgggtccagtttgtcaggctcggatggggcgttaaccatacgcatcaacggcgcccattcgca

SEQ ID NO: 33 - DNA, 151 bp, Artificial Sequence
  Synthetic oligonucleotide
ctcgccgaggagcgctgcaacaggattgttgagtagagtgtgagctccgtaactagttacatcgcaagatgtaactgaatgaaatggtgaaggacgggtccatcctgccaggctcggcgattatcgggacctttaaaccaacggcgctcgg

SEQ ID NO: 34 - DNA, 154 bp, Artificial Sequence
  Synthetic oligonucleotide
cgccccgaggagcgctgcaacagggttgttgagtagagtgtgagctccgtaactagttacatcgcaagatgtaactgaatgaaatggtgaaggacgggtccaccctgccaggctcggggatggcgccccatcgttccaacggcgctcagcgaaa

SEQ ID NO: 35 - DNA, 143 bp, Artificial Sequence
  Synthetic oligonucleotide
cctgccgaggagcgctgcgacccgattgttgagtagagtgtgagctccgtaactagttacatcgcaagatgtaactgaatgaaatggtgaaggacgggtccaccgggccaggctcggcaaccgaacaacggcgctcgcaaacc

SEQ ID NO: 36 - DNA, 151 bp, Artificial Sequence
  Synthetic oligonucleotide
cagaccgaggagcgctgcaacgggcttgttgagtagagtgtgagctccgtaactagttacatcgcaagatgtaactgaatgaaatggtgaaggacgggtccatcccgcgaggctcggcaaggaaggatcgctacaacggcgctcgttcatt

SEQ ID NO: 37 - DNA, 140 bp, Artificial Sequence
  Synthetic oligonucleotide
tgccgaggagcgctgcgacccttttgttgagtagagtgtgagctccgtaactagttacatcgcaagatgtaactgaatgaaatggtgaaggacgggtccagggggccaggctcggcaatgatcaacggcgctcgcaaacc

SEQ ID NO: 38 - DNA, 142 bp, Artificial Sequence
  Synthetic oligonucleotide
tcttccaaggagcgttgcagtcggcttgttgagtagagtgtgagctccgtaactagttacatcgcaagatgtaactgaatgaaatggtgaaggacgggtccagccggtcaggcttggatgaccccaacgacgctcacctgat

SEQ ID NO: 39 - DNA, 151 bp, Artificial Sequence
  Synthetic oligonucleotide
actttcgaggagcgttgcaactcccttgttgagtagagtgtgagctccgtaactagttacatcgcaagatgtaactgaatgaaatggtgaaggacgggtccagggagccaggctcgaaagtcatcccccgatgcaacggcgctcacccgca

SEQ ID NO: 40 - DNA, 142 bp, Artificial Sequence
  Synthetic oligonucleotide
caatccgaggagcgctgcagggccgttgttgagtagagtgtgagctccgtaactagttacatcgcaagatgtaactgaatgaaatggtgaaggacgggtccacggtttcaggctcggattccttaaacggcgctcatccgac

SEQ ID NO: 41 - DNA, 161 bp, Artificial Sequence
  Synthetic oligonucleotide
ttccgaggagcgttgcagctcgttgttgagtagagtgtgagctccgtaactagttacatcgcaagatgtaactgaatgaaatggtgaaggacgggtccacgagtgaggctcggagcacgcggccctgatggggctgcgacagtcaacggcgctcacccact

SEQ ID NO: 42 - DNA, 147 bp, Artificial Sequence
  Synthetic oligonucleotide
gacttcgaggagcgctgcgaggccgttgttgagtagagtgtgagctccgtaactagttacatcgcaagatgtaactgaatgaaatggtgaaggacgggtccacggccccaggctcgaagcggaatcacccaacggcgctcacctgta

SEQ ID NO: 43 - DNA, 145 bp, Artificial Sequence
  Synthetic oligonucleotide
cttcctgaggagcgttgcaacccacttgttgagtagagtgtgagctccgtaactagttacatcgcaagatgtaactgaatgaaatggtgaaggacgggtccagtggaccaggctcaggacttcattgcaaccacgctcacccgca

SEQ ID NO: 44 - DNA, 152 bp, Artificial Sequence
  Synthetic oligonucleotide
cgccccgaggagcgctgcaacaggttgttgagtagagtgtgagctccgtaactagttacatcgcaagatgtaactgaatgaaatggtgaaggacgggtccacctgccaggctcggggatggcgccccatcgttccaacggcgctcagcgaaa

SEQ ID NO: 45 - DNA, 141 bp, Artificial Sequence
  Synthetic oligonucleotide
cctgccgaggagcgctgcgacccgttgttgagtagagtgtgagctccgtaactagttacatcgcaagatgtaactgaatgaaatggtgaaggacgggtccacgggccaggctcggcaaccgaacaacggcgctcgcaaacc

SEQ ID NO: 46 - DNA, 138 bp, Artificial Sequence
  Synthetic oligonucleotide
tttccgaggagcgttgcgacgggttgttgagtagagtgtgagctccgtaactagttacatcgcaagatgtaactgaatgaaatggtgaaggacgggtccacccgccaggctcggaaatcttcaccaggcggtggcccg

SEQ ID NO: 47 - DNA, 140 bp, Artificial Sequence
  Synthetic oligonucleotide
tttccgaggagcgttgcgacgggcttgttgagtagagtgtgagctccgtaactagttacatcgcaagatgtaactgaatgaaatggtgaaggacgggtccagcccgccaggctcggaaatcttcaccaggcggtggcccg

SEQ ID NO: 48 - DNA, 140 bp, Artificial Sequence
  Synthetic oligonucleotide
gtttccgaggagcgttgcgacgggttgttgagtagagtgtgagctccgtaactagttacatcgcaagatgtaactgaatgaaatggtgaaggacgggtccacccgccaggctcggaaatggtcaaccgagcggtgtgtct

SEQ ID NO: 49 - DNA, 142 bp, Artificial Sequence
  Synthetic oligonucleotide
gtttccgaggagcgttgcgacgggcttgttgagtagagtgtgagctccgtaactagttacatcgcaagatgtaactgaatgaaatggtgaaggacgggtccagcccgccaggctcggaaatggtcaaccgagcggtgtgtct

SEQ ID NO: 50 - DNA, 140 bp, Artificial Sequence
  Synthetic oligonucleotide
caatccgaggagcgctgcagggccttgttgagtagagtgtgagctccgtaactagttacatcgcaagatgtaactgaatgaaatggtgaaggacgggtccaggtttcaggctcggattccttaaacggcgctcatccgac

SEQ ID NO: 51 - DNA, 142 bp, Artificial Sequence; identical to SEQ ID NO: 40
  Synthetic oligonucleotide

SEQ ID NO: 52 - DNA, 144 bp, Artificial Sequence
  Synthetic oligonucleotide
ccatccgaggagcgttgcagcgttttgttgagtagagtgtgagctccgtaactagttacatcgcaagatgtaactgaatgaaatggtgaaggacgggtccaggcgtgaggctcggatttcatcccgcaacgacgctcgcccacg

SEQ ID NO: 53 - DNA, 146 bp, Artificial Sequence
  Synthetic oligonucleotide
ccatccgaggagcgttgcagcgttcttgttgagtagagtgtgagctccgtaactagttacatcgcaagatgtaactgaatgaaatggtgaaggacgggtccagggcgtgaggctcggatttcatcccgcaacgacgctcgcccacg

SEQ ID NO: 54 - DNA, 140 bp, Artificial Sequence
  Synthetic oligonucleotide
cactccgaggagcgttgcaacggattgttgagtagagtgtgagctccgtaactagttacatcgcaagatgtaactgaatgaaatggtgaaggacgggtccatccgccaggctcggaatgtttcaacggcgctcgcagttc

SEQ ID NO: 55 - DNA, 142 bp, Artificial Sequence
  Synthetic oligonucleotide
cactccgaggagcgttgcaacggatttgttgagtagagtgtgagctccgtaactagttacatcgcaagatgtaactgaatgaaatggtgaaggacgggtccattccgccaggctcggaatgtttcaacggcgctcgcagttc

SEQ ID NO: 56 - DNA, 144 bp, Artificial Sequence
  Synthetic oligonucleotide
ctctccgaggagcgttgcagcggcttgttgagtagagtgtgagctccgtaactagttacatcgcaagatgtaactgaatgaaatggtgaaggacgggtccagtcgcgaggctcggagtcatacctgcaacgacgctcgcccacg

SEQ ID NO: 57 - DNA, 146 bp, Artificial Sequence
  Synthetic oligonucleotide
ctctccgaggagcgttgcagcggccttgttgagtagagtgtgagctccgtaactagttacatcgcaagatgtaactgaatgaaatggtgaaggacgggtccaggtcgcgaggctcggagtcatacctgcaacgacgctcgcccacg

SEQ ID NO: 58 - DNA, 138 bp, Artificial Sequence
  Synthetic oligonucleotide
tgccgaggagcgctgcgaccctttgttgagtagagtgtgagctccgtaactagttacatcgcaagatgtaactgaatgaaatggtgaaggacgggtccaggggccaggctcggcaatgatcaacggcgctcgcaaacc

SEQ ID NO: 59 - DNA, 145 bp, Artificial Sequence
  Synthetic oligonucleotide
ccgttcgaggagcgttgcgacacattgttgagtagagtgtgagctccgtaactagttacatcgcaagatgtaactgaatgaaatggtgaaggacgggtccaggtgctaggctcgaacaagaaataaacaacggcgctcacgcttc

SEQ ID NO: 60 - DNA, 147 bp, Artificial Sequence
  Synthetic oligonucleotide
ccgttcgaggagcgttgcgacacacttgttgagtagagtgtgagctccgtaactagttacatcgcaagatgtaactgaatgaaatggtgaaggacgggtccagggtgctaggctcgaacaagaaataaacaacggcgctcacgcttc

SEQ ID NO: 61 - DNA, 150 bp, Artificial Sequence
  Synthetic oligonucleotide
gcgccgaggagcgctgcgacggcttgttgagtagagtgtgagctccgtaactagttacatcgcaagatgtaactgaatgaaatggtgaaggacgggtccagccgccaggctcggcggggacaatcggttttccaacggcgctctgtttat

SEQ ID NO: 62 - DNA, 152 bp, Artificial Sequence
  Synthetic oligonucleotide
gcgccgaggagcgctgcgacggccttgttgagtagagtgtgagctccgtaactagttacatcgcaagatgtaactgaatgaaatggtgaaggacgggtccaggccgccaggctcggcggggacaatcggttttccaacggcgctctgtttat

SEQ ID NO: 63 - DNA, 149 bp, Artificial Sequence
  Synthetic oligonucleotide
actttcgaggagcgttgcaactccttgttgagtagagtgtgagctccgtaactagttacatcgcaagatgtaactgaatgaaatggtgaaggacgggtccaggagccaggctcgaaagtcatcccccgatgcaacggcgctcacccgca

SEQ ID NO: 64 - DNA, 145 bp, Artificial Sequence
  Synthetic oligonucleotide
gacttcgaggagcgctgcgaggccttgttgagtagagtgtgagctccgtaactagttacatcgcaagatgtaactgaatgaaatggtgaaggacgggtccaggccccaggctcgaagcggaatcacccaacggcgctcacctgta

SEQ ID NO: 65 - DNA, 154 bp, Artificial Sequence
  Synthetic oligonucleotide
ctcgccgaggagcgctgcaacaggttgttgagtagagtgtgagctccgtaactagttacatcgcaagatgtaactgaatgaaatggtgaaggacgggtccacctgccaggctcggcgattatcgggacctttaaaccaacggcgctcggttcag

SEQ ID NO: 66 - DNA, 151 bp, Artificial Sequence
  Synthetic oligonucleotide
ttgtaccgaggagcgctgcaacggtttgttgagtagagtgtgagctccgtaactagttacatcgcaagatgtaactgaatgaaatggtgaaggacgggtccactcgtgaggctcggacaaatgaacaacccaacaacggcgctctttcact

SEQ ID NO: 67 - DNA, 153 bp, Artificial Sequence
  Synthetic oligonucleotide
ttgtaccgaggagcgctgcaacggtcttgttgagtagagtgtgagctccgtaactagttacatcgcaagatgtaactgaatgaaatggtgaaggacgggtccatctcgtgaggctcggacaaatgaacaacccaacaacggcgctctttcact

SEQ ID NO: 68 - DNA, 149 bp, Artificial Sequence
  Synthetic oligonucleotide
cagaccgaggagcgctgcaacgggttgttgagtagagtgtgagctccgtaactagttacatcgcaagatgtaactgaatgaaatggtgaaggacgggtccacccgcgaggctcggcaaggaaggatcgctacaacggcgctcgttcatt

SEQ ID NO: 69 - DNA, 155 bp, Artificial Sequence
  Synthetic oligonucleotide
ccgtccgaggggcgctgcagcaggttgttgagtagagtgtgagctccgtaactagttacatcgcaagatgtaactgaatgaaatggtgaaggacgggtccatttgtcaggctcggatggggcgttaaccatacgcatcaacggcgcccattcgca

SEQ ID NO: 70 - DNA, 143 bp, Artificial Sequence
  Synthetic oligonucleotide
cttcctgaggagcgttgcaacccattgttgagtagagtgtgagctccgtaactagttacatcgcaagatgtaactgaatgaaatggtgaaggacgggtccatggaccaggctcaggacttcattgcaaccacgctcacccgca

SEQ ID NO: 71 - DNA, 174 bp, Artificial Sequence
  Synthetic oligonucleotide
ccgttcgaggggcgctgcagcaggttgttgagtagagtgtgagctccgtaactagttacatcgcaagatgtaactgaatgaaatggtgaaggacgggtccacctgtcaggctcgaacggagcgcgctctcaggccggcctcgccggtctgtcaacgcaccaacggcgcccattc

SEQ ID NO: 72 - DNA, 176 bp, Artificial Sequence
  Synthetic oligonucleotide
ccgttcgaggggcgctgcagcaggcttgttgagtagagtgtgagctccgtaactagttacatcgcaagatgtaactgaatgaaatggtgaaggacgggtccagcctgtcaggctcgaacggagcgcgctctcaggccggcctcgccggtctgtcaacgcaccaacggcgcccattc

SEQ ID NO: 73 - DNA, 174 bp, Artificial Sequence
  Synthetic oligonucleotide
ctgtccgaggggcgctgcagcaggttgttgagtagagtgtgagctccgtaactagttacatcgcaagatgtaactgaatgaaatggtgaaggacgggtccacctgtcaggctcggatggggcgttgttggtacagggctccctgtatggacactaaacgcacaacggcgcccat

SEQ ID NO: 74 - DNA, 176 bp, Artificial Sequence
  Synthetic oligonucleotide
ctgtccgaggggcgctgcagcaggcttgttgagtagagtgtgagctccgtaactagttacatcgcaagatgtaactgaatgaaatggtgaaggacgggtccaacctgtcaggctcggatggggcgttgttggtacagggctccctgtatggacactaaacgcacaacggcgcccat

SEQ ID NO: 75 - DNA, 169 bp, Artificial Sequence
  Synthetic oligonucleotide
ctgtccgaggggcgctgcagcaggttgttgagtagagtgtgagctccgtaactagttacatcgcaagatgtaactgaatgaaatggtgaaggacgggtccacctgtcaggctcggatggggcgttgctcgctcccgcgagcgcttaacgcacaacggcgcccattcgca

SEQ ID NO: 76 - DNA, 171 bp, Artificial Sequence
  Synthetic oligonucleotide
ctgtccgaggggcgctgcagcaggcttgttgagtagagtgtgagctccgtaactagttacatcgcaagatgtaactgaatgaaatggtgaaggacgggtccagcctgtcaggctcggatggggcgttgctcgctcccgcgagcgcttaacgcacaacggcgcccattcgca

SEQ ID NO: 77 - DNA, 156 bp, Artificial Sequence
  Synthetic oligonucleotide
ccacccgaggggcgctgcagcaggttgttgagtagagtgtgagctccgtaactagttacatcgcaagatgtaactgaatgaaatggtgaaggacgggtccacctgtcaggctcggatggggcgtttcctcgaacgcatcaacggcgcccattcgca

SEQ ID NO: 78 - DNA, 158 bp, Artificial Sequence
  Synthetic oligonucleotide
ccacccgaggggcgctgcagcaggtttgttgagtagagtgtgagctccgtaactagttacatcgcaagatgtaactgaatgaaatggtgaaggacgggtccaacctgtcaggctcggatggggcgtttcctcgaacgcatcaacggcgcccattcgca

SEQ ID NO: 79 - DNA, 181 bp, Artificial Sequence
  Synthetic oligonucleotide
cctgtccgaggggcgctgcagcagttgttgagtagagtgtgagctccgtaactagttacatcgcaagatgtaactgaatgaaatggtgaaggacgggtccactgtcaggctcggatggggcgttgtttgtcaggttgatcacagccggcaagccttaaacgcacaacggcgcccattcgca

SEQ ID NO: 80 - DNA, 183 bp, Artificial Sequence
  Synthetic oligonucleotide
cctgtccgaggggcgctgcagcaggttgttgagtagagtgtgagctccgtaactagttacatcgcaagatgtaactgaatgaaatggtgaaggacgggtccacctgtcaggctcggatggggcgttgtttgtcaggttgatcacagccggcaagccttaaacgcacaacggcgcccattcgca

SEQ ID NO: 81 - DNA, 159 bp, Artificial Sequence
  Synthetic oligonucleotide
ttccgaggagcgttgcagctcttgttgagtagagtgtgagctccgtaactagttacatcgcaagatgtaactgaatgaaatggtgaaggacgggtccagagtgaggctcggagcacgcggccctgatggggctgcgacagtcaacggcgctcacccact

SEQ ID NO: 82 - DNA, 161 bp, Artificial Sequence; identical to SEQ ID NO: 41
  Synthetic oligonucleotide

SEQ ID NO: 83 - DNA, 140 bp, Artificial Sequence
  Synthetic oligonucleotide
tcttccaaggagcgttgcagtcggttgttgagtagagtgtgagctccgtaactagttacatcgcaagatgtaactgaatgaaatggtgaaggacgggtccaccggtcaggcttggatgaccccaacgacgctcacctgat

SEQ ID NO: 84 - DNA, 137 bp, Artificial Sequence
  Synthetic oligonucleotide
ttccgaggagcgttgcaacggcttgttgagtagagtgtgagctccgtaactagttacatcgcaagatgtaactgaatgaaatggtgaaggacgggtccagccgccaggctcggaagttcaaacggcgctcgcactat

SEQ ID NO: 85 - DNA, 139 bp, Artificial Sequence
  Synthetic oligonucleotide
ttccgaggagcgttgcaacggccttgttgagtagagtgtgagctccgtaactagttacatcgcaagatgtaactgaatgaaatggtgaaggacgggtccaggccgccaggctcggaagttcaaacggcgctcgcactat

SEQ ID NO: 86 - DNA, 148 bp, Artificial Sequence
  Synthetic oligonucleotide
ctgccgaggggcgctgcgaccggttgttgagtagagtgtgagctccgtaactagttacatcgcaagatgtaactgaatgaaatggtgaaggacgggtccaccggccaggctcggccaggtggtactgcaacaacggcgcccggccaga

SEQ ID NO: 87 - DNA, 150 bp, Artificial Sequence
  Synthetic oligonucleotide
ctgccgaggggcgctgcgaccggattgttgagtagagtgtgagctccgtaactagttacatcgcaagatgtaactgaatgaaatggtgaaggacgggtccagccggccaggctcggccaggtggtactgcaacaacggcgcccggccaga

SEQ ID NO: 88 - DNA, 140 bp, Artificial Sequence
  Synthetic oligonucleotide
caacctgaggagcgctgcaacgggttgttgagtagagtgtgagctccgtaactagttacatcgcaagatgtaactgaatgaaatggtgaaggacgggtccacccgccaggctcaggttcatgtaacggcgctcgctgact

SEQ ID NO: 89 - DNA, 142 bp, Artificial Sequence
  Synthetic oligonucleotide
caacctgaggagcgctgcaacggggttgttgagtagagtgtgagctccgtaactagttacatcgcaagatgtaactgaatgaaatggtgaaggacgggtccatcccgccaggctcaggttcatgtaacggcgctcgctgact

SEQ ID NO: 90 - DNA, 144 bp, Artificial Sequence
  Synthetic oligonucleotide
cgctccgaggagcgttgcagcgccttgttgagtagagtgtgagctccgtaactagttacatcgcaagatgtaactgaatgaaatggtgaaggacgggtccaggcgtgaggctcgggcatcatctggcaacgacgctcatccaca

SEQ ID NO: 91 - DNA, 146 bp, Artificial Sequence
  Synthetic oligonucleotide
cgctccgaggagcgttgcagcgcctttgttgagtagagtgtgagctccgtaactagttacatcgcaagatgtaactgaatgaaatggtgaaggacgggtccagggcgtgaggctcgggcatcatctggcaacgacgctcatccaca

SEQ ID NO: 92 - DNA, 95 bp, Artificial Sequence
  Synthetic oligonucleotide
gatgtaactgaatgaaatggtgaaggacgggtccagtaggctgcttcggcagcctacttgttgagtagagtgtgagctccgtaactagttacatc

SEQ ID NO: 93 - DNA, 95 bp, Artificial Sequence
  Synthetic oligonucleotide
gcagcctacttgttgagtagagtgtgagctccgtaactagttacatcgcaagatgtaactgaatgaaatggtgaaggacgggtccagtaggctgc

SEQ ID NO: 94 - DNA, 152 bp, Artificial Sequence
  Synthetic oligonucleotide
cgccccgaggagcgctgcaacaggttgttgagtagagtgtgagctccgtaactagttacatcgcaagatgtaactgaatgaaatggtgaaggacgggtccacctgcctcgctcggggatggcgccccatcgttccaacggcgctcagcgaaa

SEQ ID NO: 95 - DNA, 152 bp, Artificial Sequence
  Synthetic oligonucleotide
cgccccgaggagcgctccaacaggttgttgagtagagtgtgagctccgtaactagttacatcgcaagatgtaactgaatgaaatggtgaaggacgggtccacctgccaggctcggggatggcgccccatcgttccaacggcgctcagcgaaa

SEQ ID NO: 96 - DNA, 154 bp, Artificial Sequence
  Synthetic oligonucleotide
ctcgccgaggagcgctgcaacaggttgttgagtagagtgtgagctccgtaactagttacatcgcaagatgtaactgaatgaaatggtgaaggacgggtccacctgcctcgctcggcgattatcgggacctttaaaccaacggcgctcggttcag

SEQ ID NO: 97 - DNA, 154 bp, Artificial Sequence
  Synthetic oligonucleotide
ctcgccgaggagcgctccaacaggttgttgagtagagtgtgagctccgtaactagttacatcgcaagatgtaactgaatgaaatggtgaaggacgggtccacctgccaggctcggcgattatcgggacctttaaaccaacggcgctcggttcag

SEQ ID NO: 98 - DNA, 151 bp, Artificial Sequence
  Synthetic oligonucleotide
actttcgaggagcgttgcaactcccttgttgagtagagtgtgagctccgtaactagttacatcgcaagatgtaactgaatgaaatggtgaaggacgggtccagggagcctcgctcgaaagtcatcccccgatgcaacggcgctcacccgca

SEQ ID NO: 99 - DNA, 151 bp, Artificial Sequence
  Synthetic oligonucleotide
actttcgaggagcgttccaactcccttgttgagtagagtgtgagctccgtaactagttacatcgcaagatgtaactgaatgaaatggtgaaggacgggtccagggagccaggctcgaaagtcatcccccgatgcaacggcgctcacccgca

SEQ ID NO: 100 - DNA, 24 bp, Artificial Sequence
  Synthetic oligonucleotide
ccaagtaatacgactcactatagg

SEQ ID NO: 101 - DNA, 47 bp, Artificial Sequence
  Synthetic oligonucleotide
tagcataaccccttggggcctctaaacgggtcttgaggggttttttg

SEQ ID NO: 102 - RNA, 16 nt, Artificial Sequence
  Synthetic oligonucleotide
  misc_feature 1, 2, 11: n = C or U
  misc_feature 3, 7, 15, 16: n = A or G
nnnaggngcgnugcnn

SEQ ID NO: 103 - RNA, 10 nt, Artificial Sequence
  Synthetic oligonucleotide
  misc_feature 1, 8: n = U or C
  misc_feature 9, 10: n = A or G
ncaggcunnn

SEQ ID NO: 104 - RNA, 12 nt, Artificial Sequence
  Synthetic oligonucleotide
  misc_feature 10: n = U or C
  misc_feature 6, 12: n = A or G
caacgncgcncn

SEQ ID NO: 105 - RNA, 16 nt, Artificial Sequence
  Synthetic oligonucleotide
ccgaggggcgcugcag

SEQ ID NO: 106 - RNA, 10 nt, Artificial Sequence
  Synthetic oligonucleotide
ucaggcucgg

SEQ ID NO: 107 - RNA, 12 nt, Artificial Sequence
  Synthetic oligonucleotide
caacggcgccca

SEQ ID NO: 108 - RNA, 95 nt, Artificial Sequence
  Synthetic oligonucleotide
gcagccuacuuguugaguagagugugagcuccguaacuaguuacaucgcaagauguaacugaaugaaauggugaaggacggguccaguaggcugc

SEQ ID NO: 109 - RNA, 152 nt, Artificial Sequence
  Synthetic oligonucleotide
cgccccgaggagcgcugcaacagguuguugaguagagugugagcuccguaacuaguuacaucgcaagauguaacugaaugaaauggugaaggacggguccaccugccaggcucggggauggcgccccaucguuccaacggcgcucagcgaaa

SEQ ID NO: 110 - RNA, 18 nt, Artificial Sequence
  Synthetic oligonucleotide
  misc_feature 1, 11: n = U or C
  misc_feature 7, 15, 16, 18: n = A or G
ncgaggngcgnugcnncn

SEQ ID NO: 111 - RNA, 12 nt, Artificial Sequence
  Synthetic oligonucleotide
  misc_feature 1, 3: n = U or C
  misc_feature 12: n = A or G
ngncaggcucgn

SEQ ID NO: 112 - RNA, 13 nt, Artificial Sequence
  Synthetic oligonucleotide
  misc_feature 9, 12, 13: n = T or C
  misc_feature 11: n = A or G
caaggcgcncnnn

SEQ ID NO: 113 - DNA, 33 bp, Artificial Sequence
  Synthetic oligonucleotide
gcccggatagctcagtcggtagagcagcggccg

SEQ ID NO: 114 - RNA, 8 nt, Artificial Sequence
  Synthetic oligonucleotide
aggacggg

SEQ ID NO: 115 - RNA, 11 nt, Artificial Sequence
  Synthetic oligonucleotide
guagaguguga

SEQ ID NO: 116 - RNA, 21 nt, Artificial Sequence
  Synthetic oligonucleotide
agcuccguaacuaguuacauc

SEQ ID NO: 117 - RNA, 20 nt, Artificial Sequence
  Synthetic oligonucleotide
gcuccguaacuaguuacauc

SEQ ID NO: 118 - RNA, 24 nt, Artificial Sequence
  Synthetic oligonucleotide
gauguaacugaaugaaauggugaa

SEQ ID NO: 119 - RNA, 23 nt, Artificial Sequence
  Synthetic oligonucleotide
gauguaacugaaugaaaugguga

SEQ ID NO: 120 - DNA, 11 bp, Artificial Sequence
  Synthetic oligonucleotide
gtagagtgtga

SEQ ID NO: 121 - DNA, 10 bp, Artificial Sequence
  Synthetic oligonucleotide
gtagagtgtg

SEQ ID NO: 122 - DNA, 77 bp, Artificial Sequence
  Synthetic oligonucleotide
ttgttgagtagagtgtgagctccgtaactagttacatcgcaagatgtaactgaatgaaatggtgaaggacgggtcca

SEQ ID NO: 123 - DNA, 136 bp, Artificial Sequence
  Synthetic oligonucleotide
  misc_feature 1, 2, 3, 4, 21, 22, 23, 24, 25, 103, 104, 105, 106, 107,118, 131, 132, 133, 134, 135, 136: n = A,T,C or G
  misc_feature 5, 6, 7, 15, 108, 115, 128: n = T or C
  misc_feature 7, 11, 19, 20, 116, 117, 124, 130: n = A or G
nnnnnnnaggngcgnugcnnnnnnnttgttgagtagagtgtgagctccgtaactagttacatcgcaagatgtaactgaatgaaatggtgaaggacgggtccannnnnncaggctnnnncaacgncgcncnnnnnnn

SEQ ID NO: 124 - RNA, 120 nt, Artificial Sequence
  Synthetic oligonucleotide
  misc_feature 1, 18, 96,  107, 120: n = A,U,C or G
  misc_feature 2, 3, 12, 97, 104, 105, 117: n = U or C
  misc_feature 4, 8, 16, 17, 105, 106, 113, 119: n = A or G
nnnnaggngcgnugcnnnuuguugaguagagugugagcuccguaacuaguuacaucgcaagauguaacugaaugaaauggugaaggacggguccanycaggcunnnncaacgncgcncnn

SEQ ID NO: 125 - DNA, 136 bp, Artificial Sequence
  Synthetic oligonucleotide
  misc_feature 1, 2, 3, 4, 21, 22, 23, 24, 25, 103, 104, 105, 106, 107,118, 131, 132, 133, 134, 135, 136: n = A,T,C or G
  misc_feature 5, 15, 108, 109: n = T or C
  misc_feature 19, 20, 124, 130: n = A or G
nnnnncgaggggcgntgcnnnnnnnttgttgagtagagtgtgagctccgtaactagttacatcgcaagatgtaactgaatgaaatggtgaaggacgggtccannnnnnnaggctcggncaacgncgcccnnnnnnn

SEQ ID NO: 126 - DNA, 120 bp, Artificial Sequence
  Synthetic oligonucleotide
  misc_feature 1, 18, 96, 107, 120: n = A,T,C or G
  misc_feature 2, 12, 97, 98: n = T or C
  misc_feature 16, 17, 113, 119: n = A or G
nncgaggggcgntgcnnnttgttgagtagagtgtgagctccgtaactagttacatcgcaagatgtaactgaatgaaatggtgaaggacgggtccannnaggctcggncaacgncgcccnn

SEQ ID NO: 127 - DNA, 136 bp, Artificial Sequence
  Synthetic oligonucleotide
  misc_feature 1, 2, 3, 4, 21, 22, 23, 24, 25, 103, 104, 105, 106, 107,118, 131, 132, 133, 134, 135, 136: n = A,T,C or G
  misc_feature 15, 108: n = A,T,C or G
  misc_feature 19, 20, 130: n = A,T,C or G
nnnnccgaggggcgntgcnnnnnnnttgttgagtagagtgtgagctccgtaactagttacatcgcaagatgtaactgaatgaaatggtgaaggacgggtccannnnnncaggctcggncaacggcgcccnnnnnnn

SEQ ID NO: 128 - DNA, 120 bp, Artificial Sequence
  Synthetic oligonucleotide
  misc_feature 1, 18, 96, 107, 120: n = A,T,C or G
  misc_feature 12, 97: n = T or C
  misc_feature 16, 17, 119: n = A or G
nccgaggggcgntgcnnnttgttgagtagagtgtgagctccgtaactagttacatcgcaagatgtaactgaatgaaatggtgaaggacgggtccanncaggctcggncaacggcgcccnn

SEQ ID NO: 129 - DNA, 136 bp, Artificial Sequence
  Synthetic oligonucleotide
  misc_feature 1, 2, 3, 4, 21, 22, 23, 24, 25, 103, 104, 105, 106, 107,118, 131, 132, 133, 134, 135, 136: n = A,T,C or G
  misc_feature 15, 108: n = T or C
  misc_feature 20, 130: n = A or G
nnnnccgaggggcgntgcannnnnnttgttgagtagagtgtgagctccgtaactagttacatcgcaagatgtaactgaatgaaatggtgaaggacgggtccannnnnncaggctcggncaacggcgcccnnnnnnn

SEQ ID NO: 130 - DNA, 120 bp, Artificial Sequence
  Synthetic oligonucleotide
  misc_feature 1, 18, 96, 107, 120: n = A,T,C or G
  misc_feature 12, 97: n = T or C
  misc_feature 17, 119: n = A or G
nccgaggggcgntgcannttgttgagtagagtgtgagctccgtaactagttacatcgcaagatgtaactgaatgaaatggtgaaggacgggtccanncaggctcggncaacggcgcccnn

SEQ ID NO: 131 - DNA, 24 bp, Artificial Sequence
  Synthetic oligonucleotide
gagcccgtaagcccgttcaggtcg

SEQ ID NO: 132 - protein, 26 aa, Artificial Sequence
  Synthetic polypeptide
ARTKQTARKSTGGKAPRKQLAGGKKC

SEQ ID NO: 133 - DNA, 40 bp, Artificial Sequence
  Synthetic oligonucleotide
cggccgcgggtccagggttcaagtccctgttcgggcgcca

(19) United States
(12) Patent Application Publication
(10) Pub. No.: US 2018/0267027 A1
(43) Pub. Date: Sep. 20, 2018

(54) FLUORESCENT BIOSENSOR FOR METHYLTRANSFERASE ASSAY

(71) Applicant: The Regents of the University of California, Oakland, CA (US)

(72) Inventors: Ming Hammond, Berkeley, CA (US); Yichi Su, Berkeley, CA (US); Scott F. Hickey, Berkeley, CA (US); Samantha G.L. Keyser, Alameda, CA (US)

(21) Appl. No.: 15/760,532

(22) PCT Filed: Oct. 25, 2016
(86) PCT No.: PCT/US16/58706

§ 371 Date: Mar. 15, 2018

Related U.S. Application Data
(60) Provisional application No. 62/246,953, filed on Oct. 27, 2015.

(51) Int. Cl. G01N 33/53; C12N 15/115; G01N 33/573; G01N 33/58

(52) U.S. Cl. CPC G01N 33/5308; C12N 15/115; G01N 33/573; G01N 2333/91011; C12N 2310/16; C12N 2310/3519; C12N 2310/3517; G01N 33/582

(57) ABSTRACT

A single stranded nucleic acid biosensor for S-adenosylhomocysteine (SAH) is provided. The single stranded nucleic acid may include a SAH-binding riboswitch domain comprising a P2′ stem and a contiguous Spinach aptamer domain terminated at a P2 stem that is operably connected to the P2′ stem of the SAH-binding riboswitch domain via a P2/P2′ stem comprising 5 base pairs or less. The SAH biosensor may further include a signaling chromophore specifically bound to the Spinach aptamer domain, where the sensor is configured to fluorescently activate the signaling chromophore upon specific binding of SAH to the SAH-binding riboswitch domain. Also provided are methods in which the subject SAH biosensors fmd use including methods for determining the level of SAH in a sample and methods for determining the level of methyltransferase activity in a cell. Nucleic acid constructs for the single stranded nucleic acid and host cells including the same are also provided.

### CROSS-REFERENCE

This application claims the benefit of U.S. Provisional Patent Application No. 62/246,953, filed Oct. 27, 2015, which application is incorporated herein by reference in its entirety.

### STATEMENT REGARDING FEDERALLY SPONSORED RESEARCH

This invention was made with government support under Grant Nos. OD008677 and GM066698 awarded by the National Institutes of Health. The government has certain rights in the invention.

### INTRODUCTION

Epigenetic regulation is an essential mechanism in cellular development, and dysregulation of epigenetic processes is closely linked to diseases including cancer. One epigenetic modification of interest is methylation carried out by S-adenosyl methionine (SAM) dependent methyltransferses (MTases). SAM is a universal biological cofactor that is found in all branches of life where it plays a critical role in the transfer of methyl groups to various biomolecules, including DNA, proteins and small-molecule secondary metabolites. The methylation process thus has important implications in various disease processes and applications in industrial chemical processing. This methyl transfer is catalyzed by SAM-dependent methyltransferases (MTases) which convert co-substrate SAM into S-adenosyl homocysteine (SAH). Aberrant MTase activity has been implicated in cancer, HIV infection, and diabetes, and so has been established as an important class of therapeutic target. The identification of new MTases, validation of their methylation substrates, determination of their enzymatic kinetic parameters, and development of selective inhibitors is of interest. High-throughput screening (HTS) assays are indispensable to the modern biotech and pharma enterprises for drug and biomarker discovery. A sensitive and selective HTS assay for MTase activity would be of interest these purposes.

### SUMMARY

A single stranded nucleic acid biosensor for S-adenosylhomocysteine (SAH) is provided. The single stranded nucleic acid may include a SAH-binding riboswitch domain comprising a P2′ stem and a contiguous Spinach aptamer domain terminated at a P2 stem that is operably connected to the P2′ stem of the SAH-binding riboswitch domain via a P2/P2′ stem comprising 5 base pairs or less. The SAH biosensor may further include a signaling chromophore specifically bound to the Spinach aptamer domain, where the sensor is configured to fluorescently activate the signaling chromophore upon specific binding of SAH to the SAH-binding riboswitch domain Also provided are methods in which the subject SAH biosensors find use including methods for determining the level of SAH in a sample and methods for determining the level of methyltransferase activity in a cell. Nucleic acid constructs for the single stranded nucleic acid and host cells including the same are also provided.

Aspects of the present disclosure include single stranded nucleic acids. In some embodiments, the single stranded nucleic acid includes: a S-adenosylhomocysteine (SAH)-binding riboswitch domain comprising: a 5′-terminal domain comprising the following sequence: YYRAGGRGCGYUGCRR (SEQ ID NO:102), wherein Y is C or U and R is G or A; a 3′-terminal domain comprising the following sequences: YCAGGCUYRR (SEQ ID NO:103) and CAACGRCGCYCR (SEQ ID NO: 104), wherein Y is C or U and R is G or A; and a P2′ stem; and a contiguous Spinach aptamer domain terminated at a P2 stem that is operably connected to the P2′ stem of the SAH-binding riboswitch domain via a P2/P2′ stem comprising 5 base pairs or less. In certain embodiments, the 5′-terminal domain comprises a sequence having at least 80% sequence identity to the following sequence: CCGAGGGGCGCUGCAG (SEQ ID NO: 105). In certain embodiments, the 5′-terminal domain comprises the following sequence: CCGAGGGGCGCUGCAG (SEQ ID NO: 105). In certain embodiments, the 3′-terminal domain comprises a sequence having at least 80% sequence identity to the following sequence: UCAGGCUCGG (SEQ ID NO: 106). In certain embodiments, the 3′-terminal domain comprises a sequence having at least 80% sequence identity to the following sequence: CAACGGCGCCCA (SEQ ID NO: 107). In certain embodiments, the 3′-terminal domain comprises the following sequences: UCAGGCUCGG (SEQ ID NO: 106) and CAACGGCGCCCA (SEQ ID NO: 107).

Aspects of the present disclosure include a nucleic acid construct encoding the single stranded nucleic acid. Aspects of the present disclosure include a host cell comprising the nucleic acid construct.

Aspects of the present disclosure include biosensors. In some embodiments, the biosensor includes: a single stranded nucleic acid comprising: a S-adenosylhomocysteine (SAH)-binding riboswitch domain comprising a P2′ stem; and a contiguous Spinach aptamer domain terminated at a P2 stem that is operably connected to the P2′ stem of the SAH-binding riboswitch domain via a P2/P2′ stem of 5 base pairs or less in length; and a signaling chromophore specifically bound to the Spinach aptamer domain; wherein the sensor is configured to fluorescently activate the signaling chromophore upon specific binding of SAH to the SAH-binding riboswitch domain. In certain embodiments, the fluorescence activation of the DFHBI fluorophore is by 40% or more. In certain embodiments, the biosensor is configured to specifically bind SAH with at least 10-fold stronger affinity over SAM. In certain embodiments, the biosensor includes the single stranded nucleic acid.

Aspects of the present disclosure include a method for determining the level of SAH in a sample. In some embodiments, the method includes: contacting the sample with a biosensor; and detecting fluorescence from the biosensor thereby determining the level of SAH in the sample. In certain embodiments, the determined level of SAH in the sample is independent of the level of SAM in the sample. In certain embodiments, the method further comprises determining a methyltransferase activity of the sample based on the determined level of SAH. In certain embodiments, the sample is a cellular sample.

Aspects of the present disclosure include a method for determining level of methyltransferase activity in a cell. In some embodiments, the method comprises: contacting the cell with a single stranded nucleic acid according to claim 1 and a signaling chromophore to produce a SAH biosensor in situ; and detecting fluorescence from the signaling chromophore of the SAH biosensor thereby determining the level of methyltransferase activity in the cell. In certain embodiments, the single stranded nucleic acid is expressed by the cell. In certain embodiments, the method further comprises monitoring fluorescence of the signaling chromophore upon application of a stimulus to the cell.

Aspects of the present disclosure include a kit including a single stranded nucleic acid or a nucleic acid construct encoding the single stranded nucleic acid; and one or more components selected from a signaling chromophore, SAH, SAM, a promoter, a cell, a cloning vector and an expression cassette.

### BRIEF DESCRIPTION OF THE DRAWINGS

It is understood that the drawings, described below, are for illustration purposes only. The drawings are not intended to limit the scope of the present teachings in any way.

FIG. 1 depicts a circular permutation strategy for the design of an SAH riboswitch-based fluorescent biosensor. Circular permutation of Spinach2 is achieved by removal of the P2 loop (101) and addition of the P1 tetraloop (102). Fusion of cpSpinach2 with the SAH riboswitch via a P2/P2′ transducer stem (103) generates the single stranded nucleic acid SAH biosensor including a 5′-terminal riboswitch domain (104), a cpSinach2 domain (106) and a 3′-terminal riboswitch domain (105). cpSpinach2 aptamer binds 3,5-difluoro-4-hydroxybenzylidene imidazolinone (DFHBI) and activates its fluorescence with similar performance to Spinach2 aptamer.

FIG. 2 depicts a secondary structure model of cpSpinach2 (SEQ ID NO: 108) and the chemical structure of DFHBI.

FIG. 3, panels A and B, shows: (Panel A) an in vitro analysis of dissociation constants (K_{d}) and quantum yields (Q_{Y}) for Spinach2-DFHBI and cpSpinach2-DFHBI complexes. Error bars indicate the standard deviation for 3 independent replicates and the best-fit curves are shown; and (Panel B) a graph of the results of a folding assay for determining the fraction of RNA aptamers that is properly folded for DFHBI binding. The average percentages of correctly folded RNA are indicated above each set of bars.

FIG. 4, panels A and B, shows: (Panel A) in vitro fluorescence activation and binding affinity measurements for RNA aptamers with DFHBI; (Panel B) in vitro analysis of cpSpinach2 and Spinach2 binding affinity (K_{d}) for DFHBI at 37° C. Data was collected from 3 independent replicates and the best-fit curves are shown. Background fluorescence of DFHBI (without RNA aptamer) was subtracted from all data points. Error bars indicate the standard deviation for 3 independent replicates.

FIG. 5, panels A and B, shows the results of in vivo analysis of dose-dependent fluorescence activation of DFHBI by cpSpinach2 and Spinach2 aptamers. Insets show the non-specific background fluorescence under the same conditions, noting the different y-axis scales. For panel A, error bars indicate difference between 2 independent replicates.

FIG. 6, panels A and B, shows the results of live cell fluorescence measured by flow cytometry for E. coli BL21* cells expressing plasmid encoding RNA aptamers and incubated in media containing DFHBI. All error bars represent standard deviation between three independent replicates.

FIG. 7, panels A and B, illustrates RNA-based fluorescent biosensors of interest that detect SAH selectively and with a range of binding affinities: (Panel A) Secondary structure model of Aeh1-4 biosensor (SEQ ID NO: 109) and chemical structure of SAH; and (Panel B) Ligand profile for SAH biosensors: 1, H₂O; 2, SAH; 3, SAM; 4, Adenosine; 5, D,L-homocysteine; 6, L-methionine; 7, ATP; 8, NAD+.

FIG. 8 shows in vitro analysis of biosensors binding affinity for SAH. Fraction of biosensor bound was determined by normalizing to the fluorescence signal with saturating ligand (1.0) and without ligand (0). All error bars represent standard deviation of three independent replicates.

FIG. 9A and FIG. 9B illustrate a sequence alignment (Jalview alignment) of exemplary sequences of natural SAH riboswitch aptamers used in the phylogenetic screen for SAH riboswitch-based fluorescent biosensors. Aligned putative natural P2′ stem sequences are highlighted in FIG. 9.

FIG. 10, panels A and B, shows that the results of a screen of SAH riboswitch candidates reveal 17 RNA sequences with selective response to SAH. Biosensors incorporate riboswitch aptamers with (Panel A) 4 base-pair or (Panel B) 5 base-pair P2′ stems, and are alphabetically ordered. The nomenclature is based on species origin of the sequence and the number of base pairs retained in the P2′ stem, e.g. Mxa1-5 stands for SAH riboswitch from Myxococcus xanthus, sequence 1 with 5 base pairs of P2′ stem retained. SAH biosensors selected for further characterization are indicated with solid arrows, while other responsive sequences (greater than 0.8×fluorescence increase at 100 μM SAH) are indicated with dashed arrows. For data with error bars, results shown are averaged from 2 independent replicates.

FIG. 11, panels A-C, illustrates the fluorescence activation of RNA-based biosensors Aeh1-4 (panel A), Mpe1-5 (panel B) and Nmo1-4 (panel C) for SAH versus SAM. Error bars indicate the standard deviation for 2 independent replicates.

FIG. 12 depicts use of an exemplary RNA-based fluorescent biosensor for high-throughput screening of MTase activity and inhibition. Schematic of MTase enzyme reaction and subsequent detection of SAH by fluorescent biosensors.

FIG. 13 illustrates Aeh1-4 biosensor detects methylation activity of protein lysine MTase SET7/9 (bar 5, grey). Enzyme reactions that lack one component served as negative controls (bars 1-3, white) and SAH served as a positive control (bar 4, black).

FIG. 14, panels A-C, illustrates RNA-based fluorescent biosensors of interest detect methylation activity of bacterial DNA methyltransferase M.Sssl. For the SAH positive control, 1 μM SAH was used. Error bars indicate the standard deviation for 3 independent replicates.

FIG. 15, panels A-B, shows the results of: (Panel A) sinefungin inhibition of MTase SET7/9 as measured using Aeh1-4. Percent enzyme activity was determined by normalizing to the fluorescence signal without inhibitor (100%) and without substrate (0%); and (PanelB) The Z′ scores for MTase activity and inhibition assays using Aeh1-4 were determined in 384-well format. Error bars represent standard deviation of three independent replicates (Panel A) or 16 replicates (Panel B).

FIG. 16 shows the results of a time-course of SET7/9 enzymatic activity performed using Aeh1-4 biosensor to detect SAH production. Error bars indicate the standard deviation for 3 independent replicates.

FIG. 17 shows the results of dose-dependent inhibition of SET7/9 MTase by sinefungin as characterized with 40 μM as the starting concentration of SAM. Best-fit curve is shown.

FIG. 18 illustrates the determination of Z′ score for MTase inhibition assay using Nmo 1-4 performed in HTS format. Both enzymatic reaction and fluorescent measurements were carried out in a 384-well plate. Error bars represent standard deviation of 16 independent replicates.

FIG. 19, panels A-B, depicts the detection of SAH in live E. coli by RNA-based fluorescent biosensors. (Panel A) In E. coli, SAH is catabolized by methylthioadenosine nucleosidase (MTAN) to SRH. (Panel B) Nmo1-4 and Mpe1-5 biosensors detect accumulation of SAH in E.coli BL21*MTAN-knockout strain compared to WT BL21*. Live cell fluorescence is measured by flow cytometry of cells expressing plasmids encoding negative control (DESCRIBE), cpSpinach2, active biosensors, or mutant inactive biosensors.

FIG. 20, panels A and B, depicts the detection of SAH in live E. coli by RNA-based fluorescent biosensors. (Panel A) SAH accumulation caused by BuT-DADMe-ImmA inhibition of MTAN in wild-type E.coli BL21* cells is directly detected using Nmo1-4 biosensor. (Panel B) Dynamic change of SAH levels in wild-type E. coli BL21* population upon inhibitor treatment is measured using Nmo1-4. Graph shows mean fluorescence intensities with and without inhibitor. A set of representative flow cytometry profiles also is shown, the arrow indicates increase in fluorescence. n.s. not significant; ^{**}, p <0.05 (Student's two-tailed t test: red, unpaired; black, paired). Error bars represent standard deviation of three independent biological replicates (Panel A) or 2 replicates (Panel B).

FIG. 21 depicts a representation of structural and sequence motifs of one exemplary riboswitch domain of a SAH biosensor of interest. From 5′ to 3′: YCGAGGRGCGYUGCRRCR (SEQ ID NO: 110), YGYCAGGCUCGR (SEQ ID NO: 111) and CAAGGCGCYCRYY (SEQ ID NO: 112), where Y is C or U and R is G or A.

FIG. 22, panels A and B, show results of in vitro characterization of SAH biosensors and corresponding mutants under mock physiological conditions. (Panel A) In vitro analysis of biosensor binding affinities for SAH under condition of low concentration of magnesium ion (3 mM). (Panel B) Profile of SAH RNA-based biosensors and their corresponding mutants in response to SAH. Error bars represent standard deviation of 2 independent replicates.

FIG. 23 illustrates the live cell detection of SAH accumulation in E. coli BL21*MTAN knockout strain 2 using SAH RNA-based biosensors. The number above each pair of bars represents fold change in fluorescence between wild-type and the MTAN knockout strain. Error bars represent standard deviation of 3 independent biological replicates.

FIG. 24 illustrates the live cell detection of SAH level for examining the effect of SAHH inhibitor, Adox, treatment in WT E. coli cells. Error bars represent standard deviation of 2 independent replicates.

FIG. 25 illustrates the live cell detection of SAH accumulation caused by methylthioadenosine nucleosidase (MTAN) inhibition in E. coli BL21* wild-type strain using SAH biosensors Nmo1-4 and Mpe1-5. Graph shows mean fluorescence intensities with or without MTAN inhibitor. Error bars represent standard deviation of 3 independent biological replicates.

FIG. 26, panels A and B, show selectivity profile of SAH biosensors for (Panel A) SAH and MTAN inhibitor, BuT-DADMe-ImmA, and for (Panel B) SAH and Methylthioadenosine (MTA). Error bars represent standard deviation of 2 independent replicates.

### DEFINITIONS

Before describing exemplary embodiments in greater detail, the following definitions are set forth to illustrate and define the meaning and scope of the terms used in the description. Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, N.Y. (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

As used herein, the term “aptamer” refers to a nucleic acid molecule, such as RNA or DNA (in some cases RNA), that is capable of binding to a specific molecule with high affinity and specificity (Ellington et al., Nature 346: 818-22, 1990; and Tuerk et al., Science 249: 505-10, 1990). Although aptamer in general can bind a wide variety of exemplary ligands, including, without limitation, small molecules, such as drugs, metabolites, intermediates, cofactors, transition state analogs, ions, metals, nucleic acids, polysaccharides, glycoproteins, hormones, receptors and cell surfaces (such as cell walls and cell membranes), and toxins, aptamers that may be used in the present disclosure bind target molecules such as fluorophores and SAH.

As used herein, the term “stem” refers to secondary structural feature of a single stranded nucleic acid that includes base pairing. The stem-loop structure in which a base-paired helix ends in a short unpaired loop is a common feature and is a building block for larger structural motifs, such as cloverleaf structures, which are four-helix junctions such as those found in transfer RNA. Internal loops (a short series of unpaired bases in a longer paired helix) and bulges (regions in which one strand of a helix has “extra” inserted bases with no counterparts in the opposite strand) are also frequent. A pseudoknot is a nucleic acid secondary structure containing at least two stem-loop structures in which half of one stem is intercalated between the two halves of another stem. Pseudoknots fold into knot-shaped three-dimensional conformations but are not true topological knots. The base pairing in pseudoknots is not well nested; that is, base pairs occur that “overlap” one another in sequence position.

The terms “specific binding,” “specifically binds,” and the like, refer to the ability of one binding moiety to preferentially bind directly to a target molecule relative to other molecules or moieties in a sample. In certain embodiments, the affinity between a given binding moiety and the molecule or moiety to which it specifically binds when they are specifically bound to each other in a binding complex is characterized by a K_{D} (dissociation constant) of 10⁻⁶ M or less, 10 ⁻⁷ M or less, 10⁻⁸ M or less, 10⁻⁹ M or less, 10⁻¹⁰ M or less, 10 ⁻¹¹ M or less, 10⁻¹² M or less, 10⁻¹³ M or less, 10⁻¹⁴ M or less, or 10⁻¹⁵ M or less (it is noted that these values can apply to any specific binding pair interactions mentioned elsewhere in this description, in certain embodiments).

As used herein, the terms “nucleic acid”, “oligonucleotide” and “polynucleotide” are used interchangeably and refer to a polymer of 2 or more nucleotides, such as a DNA sequence or analog thereof, or an RNA sequence or analog thereof. Nucleic acids are formed from nucleotides, including, but not limited to, the nucleotides listed herein. In some instances, the terms “nucleic acid molecule” and “polynucleotide” refer to deoxyribonucleotides or ribonucleotides and polymers thereof in either single- or double-stranded form. The term “RNA” refers to ribonucleic acid, in some cases, in single-stranded form. Unless specifically limited, the terms encompass nucleic acids/RNAs containing known analogues of natural nucleotides which have similar binding properties as the reference nucleic acid and are metabolized in a manner similar to naturally occurring nucleotides. The terms may also encompass nucleic acids/RNAs containing chemical modifications, such as modifications at the base moiety, sugar moiety, and/or phosphate backbone that tend to increase stability or half-life of the molecules in vivo. For example, these molecules may have naturally occurring phosphodiester linkages, as well as those having non-naturally occurring linkages, e.g., for stabilization purposes, or for enhancing hydrophobic interaction with protein ligands.

“Isolated” or “purified” generally refers to isolation of a substance (compound, polynucleotide, protein) such that the substance comprises the majority percent of the sample in which it resides. In some cases, in a sample a substantially purified component comprises 50%or more, such as 80%-85% or more, or 90-95% or more of the sample. Techniques for purifying polynucleotides and polypeptides of interest are well-known in the art and include, for example, ion-exchange chromatography, affinity chromatography and sedimentation according to density.

“Nucleotide” refers to naturally- and non-naturally-occurring nucleotides and nucleotide analogs. Nucleotides include, but are not limited to, adenosine, cytosine, guanosine, thymidine, uracil, 4-acetylcytosine, 8-hydroxy-N-6-methyladenosine, aziridinyl-cytosine, pseudoisocytosine, 5-(carboxyhydroxylmethyl) uracil, 5-fluorouracil, 5-bromouracil, 5-carboxymethylaminomethyl-2-thiouracil, 5-carboxy-methylaminomethyluracil, dihydrouracil, inosine, N6-iso-pentenyladenine, 1-methyladenine, 1-methylpseudouracil, 1-methylguanine, 1-methylinosine, 2,2-dimethyl-guanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonyl-methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine and 2,6-diaminopurine, 6-mercaptopurine, 5-fluorouracil, 5-iodo-2′-deoxyuridine and 6-thioguanine, cytosine exocyclic amines, substitution of 5-bromo-uracil, backbone modifications, methylations, and unusual base-pairing combinations. Additional analogs include at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil; beta-D-mannosylqueosine, 5-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

Exemplary modified base moiety may be selected from the group including, but not limited to: 5-fluorouracil, 5-bromouracil, 5-chlorouracil, 5-iodouracil, hypoxanthine, xanthine, 4-acetylcytosine, 5-(carboxyhydroxytriethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil; beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl)uracil, (acp3)w, and 2,6-diaminopurine.

Exemplary modified sugar moiety may be selected from the group including but not limited to arabinose, 2-fluoroarabinose, xylulose, and hexose.

Exemplary neutral peptide-like backbone modification include: peptide nucleic acid (PNA) (see, e.g., in Perry-O'Keefe et al. (1996) Proc. Natl. Acad. Sci. USA 93:14670 and in Eglom et al. (1993) Nature 365:566), or modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The nucleic acid may be in any physical form, e.g., linear, circular, or supercoiled.

As used herein, the term “domain” refers to a continuous or discontinuous sequence of amino acid residues or nucleotides. As used herein, the term “region” refers to a continuous sequence of amino acid residues or nucleotides.

“Complementary” refers to a nucleotide or polynucleotide sequence that hybridizes to a given nucleotide or polynucleotide sequence. For instance, for DNA, the nucleotide A is complementary to T, and vice versa, and the nucleotide C is complementary to G, and vice versa. For instance, in RNA, the nucleotide A is complementary to the nucleotide U, and vice versa, and the nucleotide C is complementary to the nucleotide G, and vice versa. Complementary nucleotides include those that undergo Watson and Crick base pairing and those that base pair in alternative modes. For instance, as used herein for RNA, the nucleotide G is complementary to the nucleotide U and vice versa, and the nucleotide A is complementary to the nucleotide G and vice versa. Therefore, in an RNA molecule, the complementary base pairs are A and U, G and C, G and U, and A and G. Other combinations, e.g., A and C, A and A, G and G, or C and U, are considered to be non-complementary base pairs.

A “complementary sequence” comprises individual nucleotides that are complementary to the individual nucleotides of a given sequence, where the complementary nucleotides are ordered such that they will pair sequentially with the nucleotides of the given sequence. Such a complementary sequence is said to be the “complement” of the given sequence.

As used herein, the terms “linker”, “linkage” or “linking group” refer to a linking moiety that connects two groups. In some instances, the linker may have a backbone of 100 atoms or less in length, e.g., 50 atoms or less in length, including 20 atoms or less in length. A linker may be a covalent bond that connects two groups or a chain of between 1 and 20 atoms in length, for example of about 1, 2, 3, 4, 5, 6, 8, 10, 12, 14, 16, 18 or 20 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. The bonds between backbone atoms may be saturated or unsaturated. In some instances, no more than one, two, or three unsaturated bonds will be present in a linker backbone. The linker may include one or more substituent groups, for example an alkyl, aryl or alkenyl group. A linker may include, without limitations, oligo(ethylene glycol), ethers, thioethers, tertiary amines, amino acid residues, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone.

In certain embodiments, the linking group comprises 1-15 carbon atoms and/or 0-6 heteroatoms. In certain embodiments, the linking group is selected from the group consisting of —(CH₂)n—C(O)—, or —C(O)(CH₂)n— or —C(O)(CH₂)n—NHC(O)—, or —C(O)(CH2)n—NHC(O)(CH₂)n—, or —(CH₂)nSCH₂C(O)—, or —(CH₂)n—C(O)NH—(CH2)n—, or —(CH₂)n—NH—C(O)—, or —(CH₂)n—NH—C(O)—(CH₂)n—, or —C(O)—(CH₂)n—, or —(CH₂)n—NH—; and n is an integer from 1 to 10, and including acid salts thereof. In certain embodiments, the linking group is —(CH₂)n—C(O)NH—(CH₂)n—, where each n is an integer from one to ten. In certain embodiments, the linking group is —(CH₂)n—C(O)NH—(CH₂)n—, where each n is one or two. In certain embodiments, the linking group is —(CH₂)n—, where n is an integer from one to ten. In certain embodiments, the linking group is —(CH₂)—. In certain embodiments, the linking group is —(CH₂)n—C(O)N(CH₂)n(CH₃)—(CH₂)n—, where each n is an integer from one to ten. In certain embodiments, the linking group is —(CH₂)n—C(O)N(CH₂)n(CH₃)—(CH₂)n—, where each n is one or two. In certain embodiments, the linking group is —(CH₂)n—C(O)N(CH₃)—(CH₂)n—, where each n is an integer from one to ten. In certain embodiments, the linking group is —(CH₂)n—C(O)N(CH₃)—(CH₂)n, where each n is one or two. In certain embodiments, the linking group comprises 10-15 carbon atoms and/or 0-6 heteroatoms. Additionally, linkers can comprise modified or unmodified nucleotides, nucleosides, polymers, sugars and other carbohydrates, polyethers, such as for example, polyethylene glycols, polyalcohols, polypropylenes, propylene glycols, mixtures of ethylene and propylene glycols, polyalkylamines, polyamines such as spermidine, polyesters such as poly(ethyl acrylate), polyphosphodiesters, and alkylenes.

A linker may be cleavable or non-cleavable. As used herein, the term “cleavable linker” refers to a linker that can be selectively cleaved to produce two products. Application of suitable cleavage conditions to a molecule containing a cleavable linker that is cleaved by the cleavage conditions will produce two cleavage products. A cleavable linker may be stable, e.g. to physiological conditions, until it is contacted with a stimulus capable of cleaving the cleavable linker.

The term “fluorophore” refers to a molecule that, when excited with light having a selected wavelength, emits light of a different wavelength, which may emit light immediately or with a delay after excitation. Fluorophores, include, without limitation, fluorescein dyes, e.g., 5-carboxyfluorescein (5-FAM), 6-carboxyfluorescein (6-FAM), 2′,4′,1,4,-tetrachlorofluorescein (TET), 2′,4′, 5′,7′,1,4-hexachlorofluorescein (HEX), and 2′,7′-dimethoxy-4′,5′-dichloro-6-carboxyfluorescein (JOE); cyanine dyes, e.g. Cy3, CYS, Cy5.5, QUASAR™ dyes etc.; dansyl derivatives; rhodamine dyes e. g. 6-carboxytetramethylrhodamine (TAMRA), CAL FLUOR dyes, tetrapropano-6-carboxyrhodamine (ROX). BODIPY fluorophores, ALEXA dyes, Oregon Green, pyrene, perylene, benzopyrene, squarine dyes, coumarin dyes, luminescent transition metal and lanthanide complexes and the like. The term fluorophore includes excimers and exciplexes of such dyes.

Other terms used herein and in the claims adopt their plain meanings as would have been understood by one of skill in the relevant art, that are not inconsistent with the usages in the instant specification. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques of described herein are those well known and commonly used in the art.

### DETAILED DESCRIPTION

As summarized above, aspects of the present disclosure include a single stranded nucleic acid biosensor for S-adenosylhomocysteine (SAH). The single stranded nucleic acid may include a SAH-binding riboswitch domain comprising a P2′ stem and a contiguous Spinach aptamer domain terminated at a P2 stem that is operably connected to the P2′ stem of the SAH-binding riboswitch domain via a P2/P2′ stem comprising 5 base pairs or less. The SAH biosensor may further include a signaling chromophore specifically bound to the Spinach aptamer domain, where the sensor is configured to fluorescently activate the signaling chromophore upon specific binding of SAH to the SAH-binding riboswitch domain. Also provided are methods in which the subject SAH biosensors find use including methods for determining the level of SAH in a sample and methods for determining the level of methyltransferase activity in a cell. Nucleic acid constructs for the single stranded nucleic acid and host cells including the same are also provided.

Before the various embodiments are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a biosensor” includes a plurality of such biosensors and reference to “the biosensor” includes reference to one or more biosensor and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Single Stranded Nucleic Acid Aptamer

Aspects of the present disclosure include a single stranded nucleic acid having a SAH binding riboswitch domain and a signaling chromophore-binding Spinach aptamer domain where the two domains are operably connected via a P2/P2′ stem. Any convenient aptamers and aptamer domains may be adapted for use in the subject biosensors. As used herein, by “operably connected” is meant the two aptamer domains are integrated into a single stranded nucleic acid, e.g., via a transducer stem located at or near to both the SAH and signaling chromophore binding sites, such that binding of the SAH target molecule to the SAH binding riboswitch domain may induce a conformational change to modulate (e.g., enhance) binding of the signaling chromophore to the Spinach aptamer domain to produce a detectable signal (e.g., a fluorescence enhancement).

The present disclosure provides a strategy for operably connecting a SAH binding riboswitch domain and a Spinach aptamer domain that includes connecting the two domains via an internal paired stem region that integrates the P2′ region of the riboswitch domain and the P2 region of the Spinach aptamer domain This strategy provides for proximity of the SAH and signaling chromophore binding sites in the integrated nucleic acid that are capable of interaction upon ligand binding, e.g., spatially, sterically, and/or via a change in structure of the ligand-bound complex.

Single stranded nucleic acids, such as aptamers, may fold to form a variety of complex secondary structures and are capable of specifically binding a target molecule. An aptamer may be obtained by in vitro selection for binding of a target molecule (e.g., a SAH ligand). Aptamers may be developed to bind particular ligands by employing known in vivo or in vitro (in some cases, in vitro) selection techniques known as SELEX (Ellington et al., Nature 346: 818-22, 1990; and Tuerk et al., Science 249, 505-10, 1990). Methods of making aptamers are also described in, for example, US-2009-0082217-A1, U.S. Pat. No. 5,582,981, PCT Publication No. WO 00/20040, U.S. Pat. No. 5,270,163, Lorsch and Szostak, Biochemistry, 33:973 (1994), Mannironi et al., Biochemistry 36:9726 (1997), Blind, Proc. Nat'l. Acad. Sci. USA 96:3606-3610 (1999), Huizenga and Szostak, Biochemistry, 34:656-665 (1995), PCT Publication Nos. WO 99/54506, WO 99/27133, WO 97/42317 and U.S. Pat. No. 5,756,291.

Aptamers have specific binding regions which are capable of forming complexes with an intended target molecule in an environment wherein other substances in the same environment are not complexed to the nucleic acid. The specificity of the binding is defined in terms of the comparative dissociation constants (K_{d}) of the aptamer for its ligand as compared to the dissociation constant of the aptamer for other materials in the environment or unrelated molecules in general. A ligand is one which binds to the aptamer with greater affinity than to unrelated material. In some cases, the K_{d} for the aptamer with respect to its ligand will be at least about 10-fold less than the K_{d} for the aptamer with unrelated material or accompanying material in the environment. In certain cases, the K_{d} will be at least 50-fold less, such as at least about 100-fold less, e.g., at least 200-fold less. An aptamer will in some cases be between 10 and 300 nucleotides in length. In certain instances, an aptamer will be between 30 and 100 nucleotides in length.

In some cases, the subject biosensor includes a nucleic acid having a SAH-binding riboswitch domain and a Spinach2 aptamer domain for binding a signaling chromophore (e.g., a fluorophore such as DFHBI or an analog thereof). In some instances, the riboswitch domain and the Spinach2 aptamer domain are operably connected to each other such that binding of SAH to the riboswitch domain activates the fluorescence of a DFHBI fluorophore bound to the Spinach2 aptamer domain

### SAH-Binding Riboswitch Domain

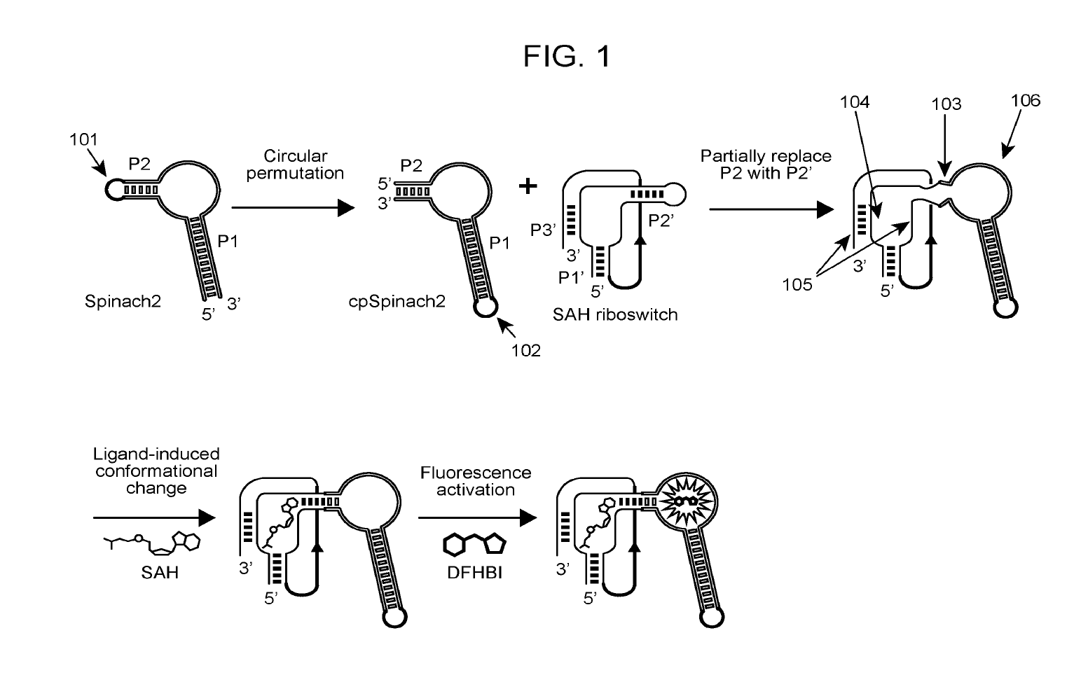

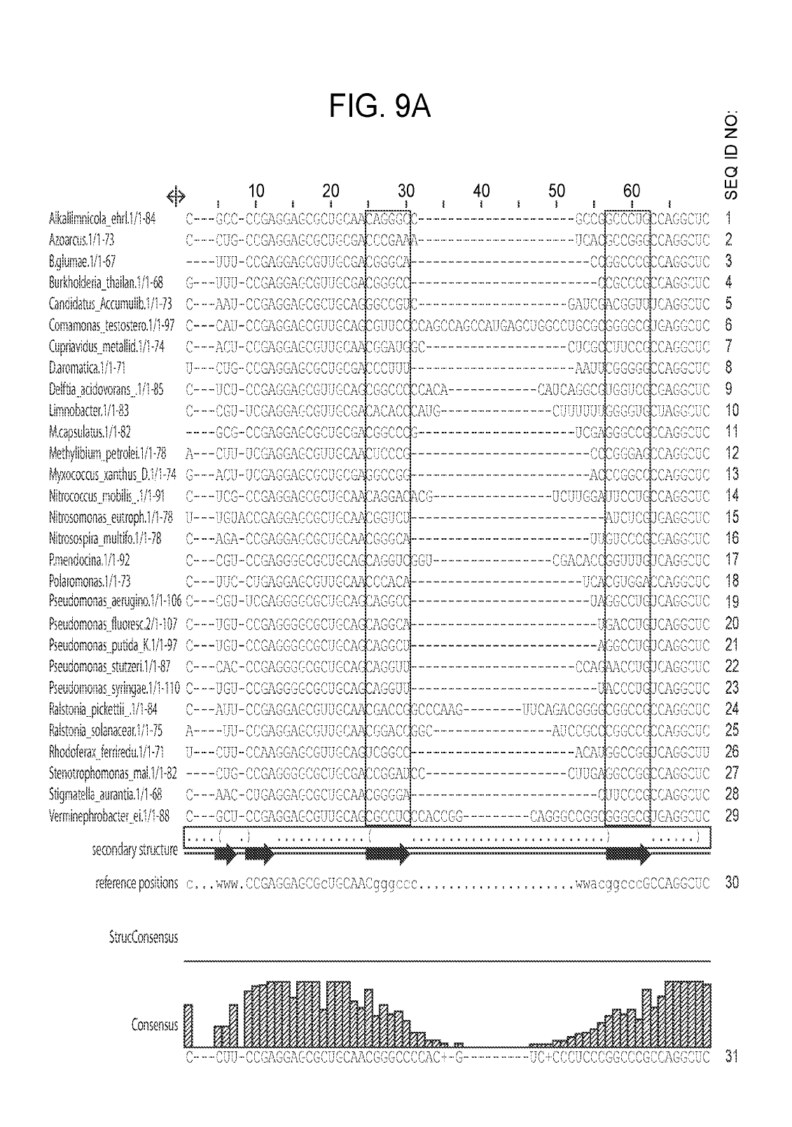

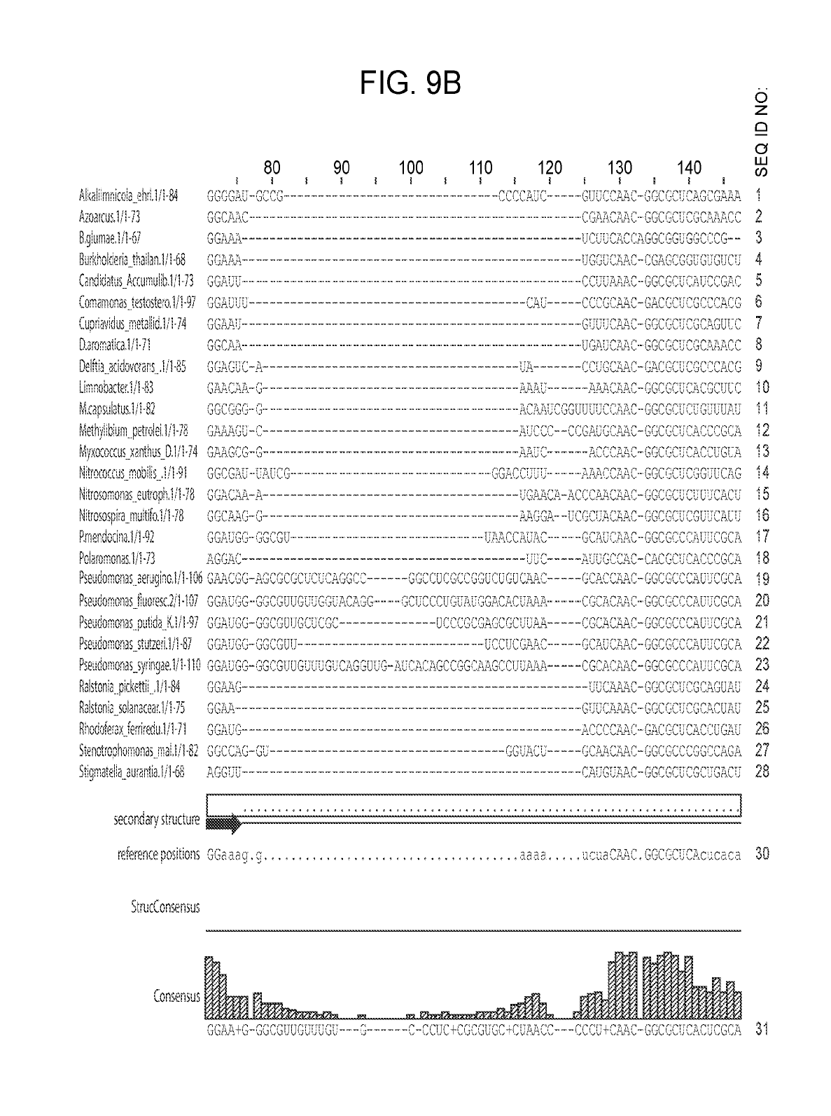

Any convenient riboswitch domains may be adapted for use in the subject nucleic acids and biosensors. By “adapted for use” is meant that a parent riboswitch of interest may be modified according to the strategy described herein (see e.g., FIG. 1) to produce a subject single stranded nucleic acid for use as a biosensor for specifically binding SAH. SAH-binding riboswitch domains of interest include, but are not limited to those described by Edwards et al., “Structural basis for recognition of S-adenosylhomocysteine by riboswitches”, RNA (2010), 16:2144-2155, the disclosure of which is herein incorporated by reference. In some embodiments, the SAH-binding riboswitch domain of interest is derived from one of the riboswitch domain sequences (SEQ ID NO: 1-31) shown in FIGS. 9A-9B.

In some embodiments, the SAH-binding riboswitch domain comprises a sequence having 80% or greater sequence identity (e.g., 85% or greater, 90% or greater, or 95% or greater sequence identity) to a sequence having one of SEQ ID NO:1-31 (see FIGS. 9A-9B) but excluding that portion of the P2′ stem region which is removed during Spinach aptamer domain integration as described herein. In some embodiments, the SAH-binding riboswitch domain comprises a 5′-terminal domain region and a discontinuous 3′-terminal domain region that together define the pseudo-knot riboswitch secondary structure, see e.g., FIG. 1, regions 104 and 105. The 5′-terminal domain region and 3′-terminal domain regions of the SAH-binding riboswitch domain define a SAH binding site in the pseudo-knot riboswitch secondary structure that provides for specific binding of SAH over SAM. The 5′-terminal domain region and 3′-terminal domain regions of the SAH-binding riboswitch domain may be arranged in the sequence of the subject nucleic acid via any convenient regions of polynucleotides, e.g., via regions of connecting sequences according to the exemplary sequences and structural motifs depicted in FIG. 21 and FIGS. 1 (104 and 105).

In some embodiments, the SAH-binding riboswitch domain comprises a 5′-terminal domain comprising the following sequence: YYRAGGRGCGYUGCRR (SEQ ID NO:102), wherein Y is C or U and R is G or A. In some embodiments, the SAH-binding riboswitch domain comprises a 3′-terminal domain comprising the following sequences: YCAGGCUYRR (SEQ ID NO:103) and CAACGRCGCYCR (SEQ ID NO: 104), wherein Y is C or U and R is G or A.

In certain embodiments, the 5′-terminal domain comprises a sequence having 80% or greater sequence identity (e.g., 85% or greater, 90% or greater sequence identity) to the following sequence: CCGAGGGGCGCUGCAG (SEQ ID NO: 105). In certain instances, the 5′-terminal domain comprises the following sequence: CCGAGGGGCGCUGCAG (SEQ ID NO: 105).

In some embodiments, the 3′-terminal domain comprises a sequence having at least 80% sequence identity (e.g., 85% or greater, 90% or greater, or 95% or greater, sequence identity) to the following sequence: UCAGGCUCGG (SEQ ID NO: 106). In certain embodiments, the 3′-terminal domain comprises a sequence having at least 80% sequence identity (e.g., 85% or greater, 90% or greater, or 95% or greater, sequence identity) to the following sequence: CAACGGCGCCCA (SEQ ID NO: 107). In certain instances, the 3′-terminal domain comprises the following sequences: UCAGGCUCGG (SEQ ID NO: 106) and CAACGGCGCCCA (SEQ ID NO: 107). Spinach aptamer domain

As used herein, the term “Spinach aptamer domain” refers to a signaling chromophore-binding aptamer domain comprising a closed P1 stem region and an open P2 stem region connected via a signaling chromophore-binding site comprising a cyclic sequence of nucleotides. The cpSpinach2 structure of FIG. 1 represents a schematic of the minimal structural features of the subject Spinach aptamer domain. By “closed P1 stem” region is meant that one end of the P1 stem region is closed by a 3′ to 5′ loop sequence, e.g., of 6 nucleotides or less, such as 5 or less, 4 or less or even less (e.g., a GCAA loop sequence), and the other end of the P1 stem is connected to the cyclic sequence defining the signaling chromophore-binding site. The P1 stem region may include 15-20 base pairs (e.g. 15, 16, 17, 18 19 or 20 base pairs) and may include a sequence of 35-55 nucleotides, such as 40-55, 45-55, 48-52 or 49 nucleotides.

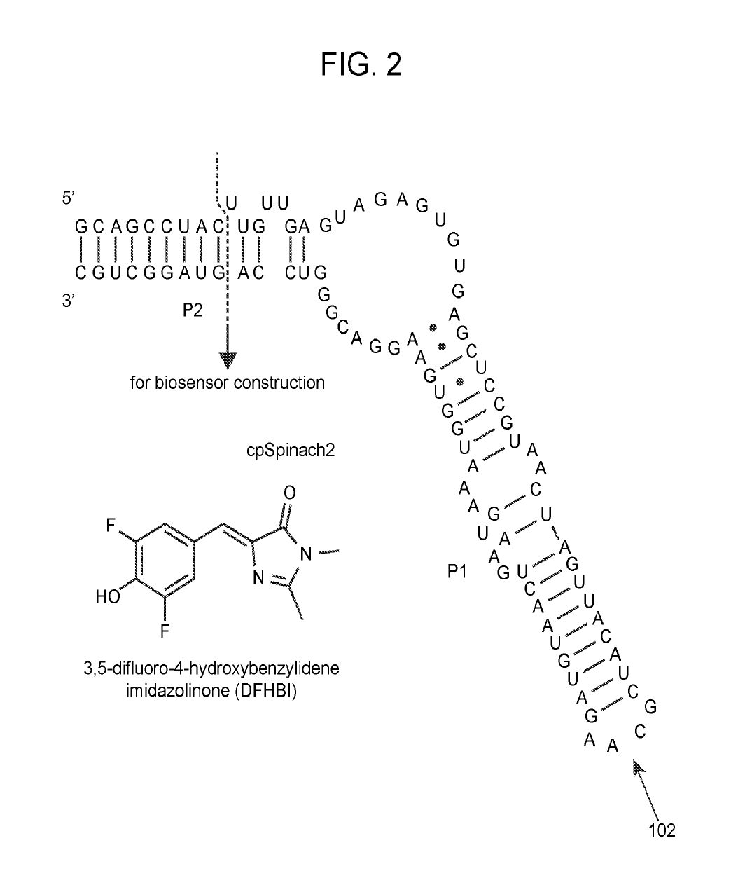

By “open P2 stem region” is meant that one end of the P2 stem has 3′ and 5′ termini and the other end of the P2 stem is connected to the cyclic sequence binding site. The P2 stem may be truncated in length (e.g., as described herein) relative to a parent Spinach aptamer. In some cases, the P2 stem has 5 or less base pairs, although not every nucleotide in the stem need by paired to a nucleotide in the opposing strand of the stem (e.g., a bulging nucleotide). The P2 stem may include 15 or fewer nucleotides in total. The signaling chromophore-binding site may comprise a discontinuous and cyclic sequence of nucleotides that includes a total of 30 nucleotides or less, such as 25 nucleotides or less, such as 17-21 nucleotides (e.g., 17, 18, 19, 20 or 21 nucleotides). FIG. 2 depicts one exemplary discontinuous cyclic sequence of nucleotides comprising a first sequence of 8 nucleotides (e.g., 5′-AGGACGGG-3′) (SEQ ID NO: 114) and a second sequence of 11 nucleotides (e.g., 5′-GUAGAGUGUGA-3′) (SEQ ID NO: 115) that together make up a discontinuous cyclic sequence which defines the signaling chromophore binding site.

Any convenient signaling chromophore-binding aptamers having may be adapted for use (e.g., as described in FIG. 1) in the subject single stranded nucleic acids. Signalling chromophore-binding aptamers which may be adapted for use (e.g., as described in FIG. 1) in the subject single stranded nucleic acid aptamers include, but are not limited to, the Spinach and Spinach2 aptamers described by Paige et al., Science 2011, 333, 642-646; Nawrocki et al., Nucleic Acids Research 2015, 43, D130-137; Strack et al. Nature Methods, 10, 1219-1224 (2013); Jaffrey et al., U.S. Publication No. 2014/0220560; and Krishnan et al., WO2015/033237, the disclosures of which are herein incorporated by reference. In some instances, the Spinach aptamer domain is derived from a Spinach2 aptamer that has been modified according to FIG. 1 (e.g., circularly modified to produce a modified cpSpinach2 aptamer). In some cases, the Spinach aptamer domain is derived from a Spinach aptamer that has been modified according to FIG. 1. In certain instances, the Spinach aptamer domain may optionally include one or more additional looped stem regions extending from the signaling chromophore binding site.

In some cases, the Spinach aptamer domain is a cpSpinach2 domain that is inserted internally in place of the natural P2 loop of the riboswitch aptamer, with a GCAA loop sequence added original 5′ and 3′ ends relative to a parent Spinach aptamer (FIG. 2). In certain embodiments, the Spinach aptamer domain is a region of a contiguous single stranded nucleic acid terminated at a P2 stem that is operably connected to the P2′ stem of the SAH-binding riboswitch domain

In some embodiments, the Spinach aptamer domain comprises the following formula (I):

5′-P2^{a}-BS^{a}-P1^{a}-loop-P1^{b}-BS^{b}-P2^{b}-3′

where:

P2^{a} and P2^{b} define a P2 stem region comprising 5 base pairs or less;

P1^{a} and P1^{b} define a P1 stem region comprising 15-20 base pairs;

loop is a sequence of 6 nucleotides or less (e.g., a GCAA loop sequence); and

BS^{a} and BS^{b} together define a discontinuous cyclic sequence of nucleotides that define a signaling chromophore-binding site (BS) comprising, in some cases, 15-23 nucleotides (e.g., 17-21 nucleotides), where BS^{a} may be a continuous sequence of 9-13 nucleotides and BS^{b} may be a continuous sequence of 6-10 nucleotides.

In some embodiments of formula (I), P2^{a} comprises the sequence UUGUUGA. In some embodiments of formula (I), P2^{b} comprises the sequence UCCA. In some embodiments of formula (I), the P2 stem comprises the following (discontinuous or continuous) sequence of base pairs: 5′-UGGA-3′ hybridized to 5′-UCCA-3′.

In some embodiments of formula (I), P1^{a} comprises the sequence:
AGCUCCGUAACUAGUUACAUC (SEQ ID NO:116). In some embodiments of formula (I), P1^{a} comprises the sequence: GCUCCGUAACUAGUUACAUC (SEQ ID NO:117). In some embodiments of formula (I), P1^{b} comprises the sequence: GAUGUAACUGAAUGAAAUGGUGAA (SEQ ID NO:118).
   In some embodiments of formula (I), P1^{b} comprises the sequence:
GAUGUAACUGAAUGAAAUGGUGA (SEQ ID NO:119). In some embodiments of formula (I), loop is a 4 or 5 nucleotide sequence, e.g., a GCAA loop sequence.

In some embodiments of formula (I), BS^{a} comprises the sequence: GTAGAGTGTGA (SEQ ID NO:120). In some embodiments of formula (I), BS^{a} comprises the sequence: GTAGAGTGTG (SEQ ID NO:121). In some embodiments of formula (I), BS^{b} comprises the sequence: AGGACGGG. In some embodiments of formula (I), BS^{b} comprises the sequence: GGACGGG.

In some embodiments of formula (I), the Spinach aptamer domain comprises a sequence having 80% or greater sequence similarity (e.g., 85% or greater, 90% or greater, 95% or greater, or 98% or greater sequence similarity) to the following sequence:
TTGTTGAGTAGAGTGTGAGCTCCGTAACTAGTTACATCGCAAGATGTAACTGAATGAAATG GTGAAGGACGGGTCCA (SEQ ID NO:122). In certain instances, when the sequence is an RNA sequence, the T nucleotides may be U nucleotides.

In some embodiments of formula (I), the Spinach aptamer domain comprises the following sequence:
TTGTTGAGTAGAGTGTGAGCTCCGTAACTAGTTACATCGCAAGATGTAACTGAATGAAATG GTGAAGGACGGGTCCA (SEQ ID NO:122). In certain instances, when the sequence is an RNA sequence, the T nucleotides may be U nucleotides.

### Signalling Chromophore

The Spinach aptamer domain may specifically bind any convenient signaling chromophore to provide for a fluorescent signal upon binding to SAH. Any convenient signaling chromophores that bind to a Spinach or Spinach2 aptamer domain may be utilized in the subject biosensors. In some instances, the signalling chromophore is a fluorophore. In some cases, the signalling chromophore is a fluorogenic compound, such that the chromophore has no significant fluorescence when it is not bound to the Spinach aptamer. The signaling chromophore may be switched to a fluorescent state of interest by binding to a Spinach aptamer domain. For example, binding of a nucleic acid molecule (e.g., as described herein) to the signaling chromophore substantially enhances fluorescence of the compound upon exposure to radiation of suitable wavelength. In some cases, the signaling chromophore is switched to a fluorescent state of interest by a SAH ligand-induced conformational change of the subject biosensor.

In certain embodiments, the signaling chromophore is a 4-hydroxybenzlidene imidazolinone (HBI) or derivative thereof, such as a chromophore described by Paige et al, Science, Vol. 333 no. 6042 pp. 642-646, 2011. In certain embodiments, the signaling chromophore is selected from one of the following:

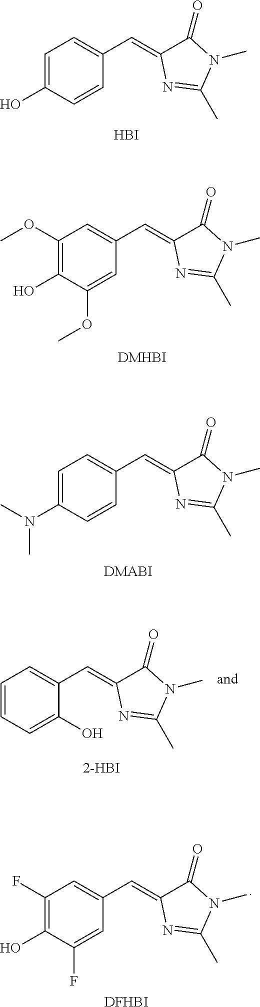

Signalling chromophores of interest that may be utilized in the subject biosensors for specifically binding to the Spinach aptamer domain include, but are not limited to, DFHBI, (Z)-4-(3,5-difluoro-4-hydroxybenzylidene)-2-methyl-1-(2,2,2-trifluoroethyl)-1H-imidazol-5(4H)-one (DFHBI-1T); (Z)-4-(3,5-difluoro-4-hydroxybenzylidene)-1-methyl-2-(trifluoromethyl)-1H-imidazol-5(4H)-one (DFHBI-2T); hydroxyl analogs of DFHBI (e.g., DFHBI-1HO, (Z)-4-(3,5-difluoro-4-hydroxy-benzylidene)-1-hydroxy-2-methyl-1H-imidazol-5(4H)-one); and methoxyl analogs of DFHBI (e.g., DFHBI-1MO, (Z)-4-(3,5-difluoro-4-hydroxybenzylidene)-1-methoxy-2-methyl-1H-imidazol-5(4H)-one); and fluorophores described by Song et al., J Am Chem Soc. 2014 Jan. 29; 136(4): 1198-1201. In some instances, the Spinach aptamer domain is capable of specifically binding (Z)-4-(3,5-difluoro-4-hydroxybenzylidene)-1,2-dimethyl-1H-imidazol-5(4H)-one (DFHBI).

In some embodiments, the signaling chromophore is a compound comprising a methyne bridge between a substituted aromatic ring system and a substituted imidazol(thi)one, oxazol(thi)one, pyrrolin(thi)one, or furan(thi)one ring, wherein binding of a nucleic acid molecule (e.g., as described herein) to the compound substantially enhances fluorescence of the compound upon exposure to radiation of suitable wavelength, such as a compound described by Jaffrey et al. in US Publication No. 20120252699, the disclosure of which is herein incorporated by reference. In certain embodiments, the signaling chromophore is a compound according to formula (I)

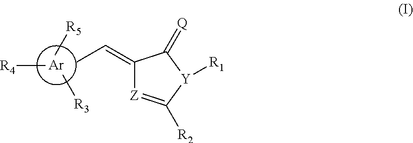

wherein, Q is S or O, Y is O or N, Z is N or C(H), Ar is an aromatic or hetero-aromatic ring system comprising one or two rings; R₁ is present when Y is N, and is a C₁₋₈ hydrocarbon or —(CH₂)ₙ—R₆ where n is an integer greater than or equal to 1; R₂ is methyl, a mono-, di-, or tri-halo methyl, oxime, O-methyl-oxime, imine, substituted or unsubstituted phenyl with up to three substituents (R₇-R₉), C₂₋₈ unsaturated hydrocarbon optionally terminated with an amine, amide, carboxylic acid, (meth)acrylate, ester, enone, oxime, O-methyl-oxime, imine, nitromethane, nitrile, ketone, mono-, di-, tri-halo, nitro, cyano, acrylonitrile, acrylonitrile-enoate, acrylonitrile-carboxylate, acrylonitrile-amide, or a second aromatic or hetero-aromatic ring; R₃-R₅ are independently selected from H, hydroxy, alkyl, alkoxy, fluoro, chloro, bromo, amino, alkylamino, dialkylamino, alkylthio, cyano, mercapto, nitro, and mono-, di-, or tri-halo methyl, ketone, and carboxylic acid; R₆ is H, a surface-reactive group, a solid surface, or a functional group that can be linked to a surface-reactive group or solid surface; R₇-R₉ are independently selected from H, hydroxy, alkyl, alkoxy, fluoro, chloro, bromo, amino, alkylamino, dialkylamino, alkylthio, cyano, mercapto, nitro, and mono-, di-, or tri-halo methyl, ketone, and carboxylic acid; and salts thereof;

In some instances, the signaling chromophore is a compound of formula (I) wherein: (i) R₃-R₅ cannot all be H; (ii) when R₁ and R₂ are methyl, and R₄ and R₅ are H, R₃ is not hydroxy, methoxy, or dimethylamino; and (iii) when R₁ is methyl, R₄ and R₅ are H, and R₃ is hydroxy, R₂ is not a conjugated hydrocarbon chain.

### P2/P2′ Stem

The SAH-binding domain and the Spinach aptamer domain may be connected as described herein by integration of the P2′ stem of the SAH-binding domain with the P2 stem of the Spinach aptamer domain. The P2′ and P2 stems may be integrated and connected in a variety of ways to provide a transducer stem capable of operably connecting the SAH-binding site with the signaling transducer binding site (e.g., the sequence BS). In some cases, the resulting integrated P2/P2′ transducer stem comprises 5 base pairs or less (e.g., 4 or 5 base pairs, NNNN or NNNNN, where N is any convenient nucleotide), where additional optional bulging nucleotides may also be included.

In some embodiments, the connecting P2/P2′ stem includes the following pair of sequences:

5′-GCTCG-3′;
and
3′-TGAGC-5′.

In some embodiments, the connecting P2/P2′ stem includes the following pair of sequences:

5′-CAGGA-3′;
and
3′-GTCCT-5′.

In some embodiments, the connecting P2/P2′ stem includes the following pair of sequences:

5′-CAGGG-3′;
and
3′-GTCCC-5′.

In some embodiments, the connecting P2/P2′ stem includes the following pair of sequences:

5′-CCCG-3′;
and
3′-GGGC-5′.

In some embodiments, the connecting P2/P2′ stem includes the following pair of sequences:

5′-CCCGA-3′;
and
3′-GGGCC-5′.

In some embodiments, the connecting P2/P2′ stem includes the following pair of sequences:

5′-CTCCC-3′;
and
3′-GAGGG-5′.

It is understood that in any of the P2/P2′ stem sequences described above, one or more additional nucleotides may be inserted as a bulging nucleotide.

In some embodiments, the single strand nucleic acid of the subject biosensor has the following sequence:
NNNNYYRAGGRGCGYUGCRRNNNNNTTGTTGAGTAGAGTGTGAGCTCCGTAACTAGTTACA TCGCAAGATGTAACTGAATGAAATGGTGAAGGACGGGTCCANNNNNYCAGGCTYRR(N)ₙCA ACGRCGCYCRNNNNNN (SEQ ID NO:123), where each N represents any convenient nucleotide; each R is independently a G or A nucleotide; each Y is independently a C or T nucleotide; and each n is independently 1 to 10.

In some embodiments, the single strand nucleic acid of the subject biosensor comprises the following sequence:
(N)nYYRAGGRGCGYUGCRR(N)mUUGUUGAGUAGAGUGUGAGCUCCGUAACUAGUUACAU CGCAAGAUGUAACUGAAUGAAAUGGUGAAGGACGGGUCCA(N)mYCAGGCUYRR(N)ₙCAAC GRCGCYCR(N)n (SEQ ID NO:124), where each N represents any convenient nucleotide; each R is independently a G or A nucleotide; each Y is independently a C or T nucleotide; each m is independently 3, 4, 5 or 6; and each n is independently 1 to 10.

In some embodiments, the single strand nucleic acid of the subject biosensor comprises the following sequence:
NNNNYCGAGGGGCGYTGCRRNNNNNTTGTTGAGTAGAGTGTGAGCTCCGTAACTAGTTACA TCGCAAGATGTAACTGAATGAAATGGTGAAGGACGGGTCCANNNNNYYAGGCTCGG(N)ₙCA ACGRCGCCCRNNNNNN (SEQ ID NO:125), where each N represents any convenient nucleotide; each R is independently a G or A nucleotide; each Y is independently a C or T nucleotide; n is 1 to 10.

In some embodiments, the single strand nucleic acid of the subject biosensor comprises the following sequence:
(N)nYCGAGGGGCGYTGCRR(N)mTTGTTGAGTAGAGTGTGAGCTCCGTAACTAGTTACATCG CAAGATGTAACTGAATGAAATGGTGAAGGACGGGTCCA(N)mYYAGGCTCGG(N)ₙCAACGRC GCCCR(N)n (SEQ ID NO:126), where each N represents any convenient nucleotide; each R is independently a G or A nucleotide; each Y is independently a C or T nucleotide; each m is independently 3, 4, 5 or 6; and each n is independently 1 to 10.

In some embodiments, the single strand nucleic acid of the subject biosensor comprises the following sequence:
NNNNCCGAGGGGCGYTGCRRNNNNNTTGTTGAGTAGAGTGTGAGCTCCGTAACTAGTTACA TCGCAAGATGTAACTGAATGAAATGGTGAAGGACGGGTCCANNNNNYCAGGCTCGG(N)ₙCA ACGGCGCCCRNNNNNN (SEQ ID NO:127), where each N represents any convenient nucleotide; each R is independently a G or A nucleotide; each Y is independently a C or T nucleotide; n is 1 to 10.

In some embodiments, the single strand nucleic acid of the subject biosensor comprises the following sequence:
(N)nCCGAGGGGCGYTGCRR(N)mTTGTTGAGTAGAGTGTGAGCTCCGTAACTAGTTACATCGC AAGATGTAACTGAATGAAATGGTGAAGGACGGGTCCA(N)mYCAGGCTCGG(N)ₙCAACGGCG CCCR(N)n (SEQ ID NO:128), where each N represents any convenient nucleotide; each R is independently a G or A nucleotide; each Y is independently a C or T nucleotide; each m is independently 3, 4, 5 or 6; and each n is independently 1 to 10.

In some embodiments, the single strand nucleic acid of the subject biosensor comprises the following sequence:
NNNNCCGAGGGGCGYTGCARNNNNNTTGTTGAGTAGAGTGTGAGCTCCGTAACTAGTTACA TCGCAAGATGTAACTGAATGAAATGGTGAAGGACGGGTCCANNNNNYCAGGCTCGG(N)ₙCA ACGGCGCCCRNNNNNN (SEQ ID NO:129), where each N represents any convenient nucleotide; each R is independently a G or A nucleotide; each Y is independently a C or T nucleotide; n is 1 to 10.

In some embodiments, the single strand nucleic acid of the subject biosensor comprises the following sequence:
(N)nCCGAGGGGCGYTGCAR(N)mTTGTTGAGTAGAGTGTGAGCTCCGTAACTAGTTACATCG CAAGATGTAACTGAATGAAATGGTGAAGGACGGGTCCA(N)mYCAGGCTCGG(N)ₙCAACGGC GCCCR(N)n (SEQ ID NO:130), where each N represents any convenient nucleotide; each R is independently a G or A nucleotide; each Y is independently a C or T nucleotide; each m is independently 3, 4, 5 or 6; and each n is 1 to 10.

In some embodiments, the single strand nucleic acid of the subject biosensor comprises a sequence having 80% or greater sequence identity (e.g., 85% or greater, 90% or greater, 95% or greater, 98% or greater sequence identity) to the following sequence (Pme):
CCGTCCGAGGGGCGCTGCAGCAGGTTTGTTGAGTAGAGTGTGAGCTCCGTAACTAGTTACA TCGCAAGATGTAACTGAATGAAATGGTGAAGGACGGGTCCAGTTTGTCAGGCTCGGATGGG GCGTTAACCATACGCATCAACGGCGCCCATTCGCA (SEQ ID NO:32). In some embodiments, the single strand nucleic acid of the subject biosensor comprises SEQ ID NO:32.

In some embodiments, the single strand nucleic acid of the subject biosensor comprises a sequence having 80% or greater sequence identity (e.g., 85% or greater, 90% or greater, 95% or greater, 98% or greater sequence identity) to the following sequence (Nmo):
CTCGCCGAGGAGCGCTGCAACAGGATTGTTGAGTAGAGTGTGAGCTCCGTAACTAGTTACA TCGCAAGATGTAACTGAATGAAATGGTGAAGGACGGGTCCATCCTGCCAGGCTCGGCGATT ATCGGGACCTTTAAACCAACGGCGCTCGG (SEQ ID NO:33). In some embodiments, the single strand nucleic acid of the subject biosensor comprises SEQ ID NO:33.

In some embodiments, the single strand nucleic acid of the subject biosensor comprises a sequence having 80% or greater sequence identity (e.g., 85% or greater, 90% or greater, 95% or greater, 98% or greater sequence identity) to the following sequence (Aeh):
CGCCCCGAGGAGCGCTGCAACAGGGTTGTTGAGTAGAGTGTGAGCTCCGTAACTAGTTACA TCGCAAGATGTAACTGAATGAAATGGTGAAGGACGGGTCCACCCTGCCAGGCTCGGGGATG GCGCCCCATCGTTCCAACGGCGCTCAGCGAAA (SEQ ID NO:34). In some embodiments, the single strand nucleic acid of the subject biosensor comprises SEQ ID NO:34.

In some embodiments, the single strand nucleic acid of the subject biosensor comprises a sequence having 80% or greater sequence identity (e.g., 85% or greater, 90% or greater, 95% or greater, 98% or greater sequence identity) to the following sequence (Azo):
CCTGCCGAGGAGCGCTGCGACCCGATTGTTGAGTAGAGTGTGAGCTCCGTAACTAGTTACA TCGCAAGATGTAACTGAATGAAATGGTGAAGGACGGGTCCACCGGGCCAGGCTCGGCAAC CGAACAACGGCGCTCGCAAACC (SEQ ID NO:35). In some embodiments, the single strand nucleic acid of the subject biosensor comprises SEQ ID NO:35.

In some embodiments, the single strand nucleic acid of the subject biosensor comprises a sequence having 80% or greater sequence identity (e.g., 85% or greater, 90% or greater, 95% or greater, 98% or greater sequence identity) to the following sequence (Nmu):
CAGACCGAGGAGCGCTGCAACGGGCTTGTTGAGTAGAGTGTGAGCTCCGTAACTAGTTACA TCGCAAGATGTAACTGAATGAAATGGTGAAGGACGGGTCCATCCCGCGAGGCTCGGCAAG GAAGGATCGCTACAACGGCGCTCGTTCATT (SEQ ID NO:36). In some embodiments, the single strand nucleic acid of the subject biosensor comprises SEQ ID NO:36.

In some embodiments, the single strand nucleic acid of the subject biosensor comprises a sequence having 80% or greater sequence identity (e.g., 85% or greater, 90% or greater, 95% or greater, 98% or greater sequence identity) to the following sequence (Dar):
TGCCGAGGAGCGCTGCGACCCTTTTGTTGAGTAGAGTGTGAGCTCCGTAACTAGTTACATCG CAAGATGTAACTGAATGAAATGGTGAAGGACGGGTCCAGGGGGCCAGGCTCGGCAATGAT CAACGGCGCTCGCAAACC (SEQ ID NO:37). In some embodiments, the single strand nucleic acid of the subject biosensor comprises SEQ ID NO:37.

In some embodiments, the single strand nucleic acid of the subject biosensor comprises a sequence having 80% or greater sequence identity (e.g., 85% or greater, 90% or greater, 95% or greater, 98% or greater sequence identity) to the following sequence (Rfem):
TCTTCCAAGGAGCGTTGCAGTCGGCTTGTTGAGTAGAGTGTGAGCTCCGTAACTAGTTACAT CGCAAGATGTAACTGAATGAAATGGTGAAGGACGGGTCCAGCCGGTCAGGCTTGGATGACC CCAACGACGCTCACCTGAT (SEQ ID NO:38). In some embodiments, the single strand nucleic acid of the subject biosensor comprises SEQ ID NO:38.

In some embodiments, the single strand nucleic acid of the subject biosensor comprises a sequence having 80% or greater sequence identity (e.g., 85% or greater, 90% or greater, 95% or greater, 98% or greater sequence identity) to the following sequence (Mpe):
ACTTTCGAGGAGCGTTGCAACTCCCTTGTTGAGTAGAGTGTGAGCTCCGTAACTAGTTACAT CGCAAGATGTAACTGAATGAAATGGTGAAGGACGGGTCCAGGGAGCCAGGCTCGAAAGTC ATCCCCCGATGCAACGGCGCTCACCCGCA (SEQ ID NO:39). In some embodiments, the single strand nucleic acid of the subject biosensor comprises SEQ ID NO:39.

In some embodiments, the single strand nucleic acid of the subject biosensor comprises a sequence having 80% or greater sequence identity (e.g., 85% or greater, 90% or greater, 95% or greater, 98% or greater sequence identity) to the following sequence (Cac):
CAATCCGAGGAGCGCTGCAGGGCCGTTGTTGAGTAGAGTGTGAGCTCCGTAACTAGTTACA TCGCAAGATGTAACTGAATGAAATGGTGAAGGACGGGTCCACGGTTTCAGGCTCGGATTCC TTAAACGGCGCTCATCCGAC (SEQ ID NO:40). In some embodiments, the single strand nucleic acid of the subject biosensor comprises SEQ ID NO:40.

In some embodiments, the single strand nucleic acid of the subject biosensor comprises a sequence having 80% or greater sequence identity (e.g., 85% or greater, 90% or greater, 95% or greater, 98% or greater sequence identity) to the following sequence (Rfea):
TTCCGAGGAGCGTTGCAGCTCGTTGTTGAGTAGAGTGTGAGCTCCGTAACTAGTTACATCGC AAGATGTAACTGAATGAAATGGTGAAGGACGGGTCCACGAGTGAGGCTCGGAGCACGCGG CCCTGATGGGGCTGCGACAGTCAACGGCGCTCACCCACT (SEQ ID NO:41). In some embodiments, the single strand nucleic acid of the subject biosensor comprises SEQ ID NO:41.

In some embodiments, the single strand nucleic acid of the subject biosensor comprises a sequence having 80% or greater sequence identity (e.g., 85% or greater, 90% or greater, 95% or greater, 98% or greater sequence identity) to the following sequence (Mxa):
GACTTCGAGGAGCGCTGCGAGGCCGTTGTTGAGTAGAGTGTGAGCTCCGTAACTAGTTACA TCGCAAGATGTAACTGAATGAAATGGTGAAGGACGGGTCCACGGCCCCAGGCTCGAAGCG GAATCACCCAACGGCGCTCACCTGTA (SEQ ID NO:42). In some embodiments, the single strand nucleic acid of the subject biosensor comprises SEQ ID NO:42.

In some embodiments, the single strand nucleic acid of the subject biosensor comprises a sequence having 80% or greater sequence identity (e.g., 85% or greater, 90% or greater, 95% or greater, 98% or greater sequence identity) to the following sequence (Pol):
CTTCCTGAGGAGCGTTGCAACCCACTTGTTGAGTAGAGTGTGAGCTCCGTAACTAGTTACAT CGCAAGATGTAACTGAATGAAATGGTGAAGGACGGGTCCAGTGGACCAGGCTCAGGACTTC ATTGCAACCACGCTCACCCGCA (SEQ ID NO:43). In some embodiments, the single strand nucleic acid of the subject biosensor comprises SEQ ID NO:43.

In some embodiments, the single strand nucleic acid of the subject biosensor comprises a sequence having 80% or greater sequence identity (e.g., 85% or greater, 90% or greater, 95% or greater, 98% or greater sequence identity) to any one of the sequences set forth in Table 3 (e.g., any one of SEQ ID NOs: 44-91).

In some embodiments, the single strand nucleic acid of the subject biosensor comprises a sequence having 80% or greater sequence identity (e.g., 85% or greater, 90% or greater, 95% or greater, 98% or greater sequence identity) to any one of the sequences set forth in Table 2 (e.g., any one of SEQ ID NOs: 94-99).

### Biosensor

Aspects of the present disclosure include biosensors for SAH. The biosensor may include a single stranded nucleic acid (e.g., as described herein); and a signaling chromophore (e.g., as described herein) specifically bound to the Spinach aptamer domain In some cases, the single stranded nucleic acid includes a S-adenosylhomocysteine (SAH)-binding riboswitch domain comprising a P2′ stem; and a contiguous Spinach aptamer domain terminated at a P2 stem that is operably connected to the P2′ stem of the SAH-binding riboswitch domain. In certain instances, the connecting P2/P2′ stem comprises 5 base pairs or less. The sensor is configured to fluorescently activate the signaling chromophore upon specific binding of SAH to the SAH-binding riboswitch domain, e.g., via a ligand activated conformational change. In some instances, the signaling chromophore is a DFHBI fluorophore.

A subject biosensor may be modified in a variety of ways depending on the application in which the biosensor finds use. Moieties of interest suitable for adapting for use in modifying the nucleic acid component of the subject biosensors include, but are not limited to, any convenient moiety suitable for attachment to the 3′ or 5′ terminal of a nucleic acid, a protein domain, a polypeptide, a peptide tag, a specific binding moiety, a polymeric moiety such as a polyethylene glycol (PEG), a carbohydrate, a dextran or a polyacrylate, a linker, a chemoselective functional group, a moiety that imparts desirable drug-like properties, a detectable label (e.g., a fluorophore), a support, a half-life extending moiety, a fatty acid, a solid support and a linker.

In some instances, the single stranded nucleic acid of the biosensor is attached to a solid support, via an optional linker. Any convenient solid supports may be utilized, including but not limited to, a bead, a microarray, a flat surface, a chromatography support, etc.

### Constructs

Nucleic acid molecules of the present disclosure (e.g., RNA) can be delivered to target cells in vitro or in vivo. A number of methods have been developed for delivering nucleic acids into cells; e.g., they can be injected directly into the tissue site, or modified nucleic acids, designed to target the desired cells can be administered systematically. Another approach utilizes a recombinant DNA construct in which the RNA or other biosensor nucleic acid is placed under the control of a strong pol III or pol II promoter. The use of such a construct to transfect target cells will result in the transcription of sufficient amounts of the subject RNA. For example, a vector or expression construct can be such that it is taken up by a target cell and directs the transcription of a subject RNA. Such a vector or expression construct can remain episomal or become chromosomally integrated, as long as it can be transcribed to produce the desired product. Such vectors can be constructed by recombinant DNA technology methods standard in the art.

Vectors can be plasmid, viral, or others known in the art, used for replication and expression in mammalian cells. A promoter may be operably linked to the sequence encoding the subject RNA. Expression of the subject encoded sequences can be by any promoter known in the art to act in mammalian, in some cases, human cells. Such promoters can be inducible or constitutive. Such promoters include but are not limited to: the SV40 early promoter region (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. USA 78:1441-1445 (1981)), the regulatory sequences of the metallothionein gene (Brinster et al, Nature 296:3942 (1982)), etc. Any type of plasmid, cosmid, YAC or viral vector can be used to prepare the recombinant DNA construct that can be introduced directly into the sample or tissue site. Alternatively, viral vectors can be used which selectively infect the desired sample or tissue, in which case administration may be accomplished by another route (e.g., systematically).

Also provided is an expression vector or construct having a coding sequence that is transcribed to produce one or more transcriptional products that produce a subject nucleic acid of interest in the treated cells. Any convenient expression vectors appropriate for producing an aptamer-regulated nucleic acid may be utilized. For example, the expression vector is selected from an episomal expression vector, an integrative expression vector, and a viral expression vector.

In certain embodiments, the expression vector can be designed to include one or more subject nucleic acid or transcript, such as in the 3′ untranslated region (3′-UTR), so as to regulate transcription, stability and/or translation of a RNA transcript in a manner dependent on a ligand. To further illustrate, the expression construct can include a coding sequence for a polypeptide such that the mRNA transcript includes both the polypeptide coding sequence as well as one or more of the RNA of the invention. In this way, expression of the construct can be controlled dependent on the ligand(s) to which the aptamer(s) bind.

### Host Cells

Aspects of the invention include providing a biosensor, or nucleic acid component thereof, in a host cell. The cells that are provided with the biosensor or nucleic acid component thereof may include a methyltransferase enzyme of interest. The cell that is provided with the biosensor may vary depending on the specific application being performed. Target cells of interest include eukaryotic cells, e.g., animal cells, where specific types of animal cells include, but are not limited to: insect, worm or mammalian cells. Various mammalian cells may be used, including, by way of example, equine, bovine, ovine, canine, feline, murine, non- human primate and human cells. Among the various species, various types of cells may be used, such as hematopoietic, neural, glial, mesenchymal, cutaneous, mucosal, stromal, muscle (including smooth muscle cells), spleen, reticulo-endothelial, epithelial, endothelial, hepatic, kidney, gastrointestinal, pulmonary, fibroblast, and other cell types. Hematopoietic cells of interest include any of the nucleated cells which may be involved with the erythroid, lymphoid or myelomonocytic lineages, as well as myoblasts and fibroblasts. Also of interest are stem and progenitor cells, such as hematopoietic, neural, stromal, muscle, hepatic, pulmonary, gastrointestinal and mesenchymal stem cells, such as ES cells, epi-ES cells and induced pluripotent stem cells (iPS cells).

In some embodiments, these cells are cells that have been engineered to include the nucleic acid component of a subject biosensor. The protocol by which the cells are engineered to include the desired nucleic acid may vary depending on one or more different considerations, such as the nature of the target cell, the nature of the biosensor, etc. The cell may include expression constructs having coding sequences for the single stranded nucleic acid component of the subject biosensor under the control of a suitable promoter. The coding sequences will vary depending on the particular nature of the nucleic acid encoded thereby, and in some cases will include at least a first domain that encodes the SAH-binding riboswitch domain and a second domain that encodes a Spinach aptamer domain. The two domains may be joined directly or linked to each other by a linking domain, e.g., a P2-P2′ transducer stem. The domains encoding the nucleic acid are in operational combination, i.e., operably linked, with requisite transcriptional mediation or regulatory element(s). Requisite transcriptional mediation elements that may be present in the expression module include promoters (including tissue specific promoters), enhancers, termination and polyadenylation signal elements, splicing signal elements, and the like. Of interest in some instances are inducible expression systems. The various expression constructs in the cell may be chromosomally integrated or maintained episomally, as desired. Accordingly, in some instances the expression constructs are chromosomally integrated in a cell. Alternatively, one or more of the expression constructs may be episomally maintained, as desired. In yet other embodiments, the nucleic acid of interest may be provided via microinjection of mRNA or proteins. The cells may be prepared using any convenient protocol, where the protocol may vary depending on nature of the cell, the location of the cell, e.g., in vitro or in vivo, etc. Where desired, vectors, such as viral vectors, may be employed to engineer the cell to express the chimeric proteins as desired. Protocols of interest include those described in published PCT application WO1999/041258, the disclosure of which protocols are herein incorporated by reference.

As desired, cells may be engineered in vitro or in vivo. For target cells that are engineered in vitro, such cells may ultimately be introduced into a host organism. Depending upon the nature of the cells, the cells may be introduced into a host organism, e g a mammal, in a wide variety of ways. In some instances, the cell comprising the biosensor system(s) is part of a multicellular organism, e.g., a transgenic animals or animal comprising a graft of such cells that comprise a biosensor system(s). Any convenient methods for generating cells having targeted gene modifications through homologous recombination may be utilized. For various techniques for transfecting mammalian cells, see Keown et al., (1990), Meth. Enzymol. 185:527-537.

### Methods

As summarized above, aspects of the present disclosure include methods for determining the level of SAH in a sample. As such, aspects of the method include, contacting the sample with a biosensor, e.g., as described above, under conditions in which SAH, if present in the sample, specifically binds to the biosensor to activate fluorescence of a bound signaling chromophore (e.g., fluorophore). The detected fluorescent signal may then be used to determine the level, e.g., concentration, of SAH in the sample. Any convenient controls and standards may be utilized in determining the level of SAH in the sample.

In some instances, the fluorescence activation of the signaling chromophore is an increase in fluorescence of 10% or more, such as, 20% or more, 30% or more, 40% or more, 50% or more, 60% or more, 70% or more, 80% or more, 90% or more, 100% or more, 120% or more, 150% or more, 200% or more, 300% or more, 500% or more, or even more relative to the fluorescence of the biosensor when it is not bound to SAH. In some instances, the fluorescence activation of the signaling chromophore is by 40% or more.

In some cases, the biosensor is configured to specifically bind SAH over SAM, e.g., with an affinity for SAH over SAM that is at least 10-fold stronger affinity, such as at least 30-fold stronger affinity, at least 100-fold stronger affinity, at least 300-fold stronger affinity, at least 1000-fold stronger affinity, e.g., as measured by a dose response curve as shown in FIG. 8.

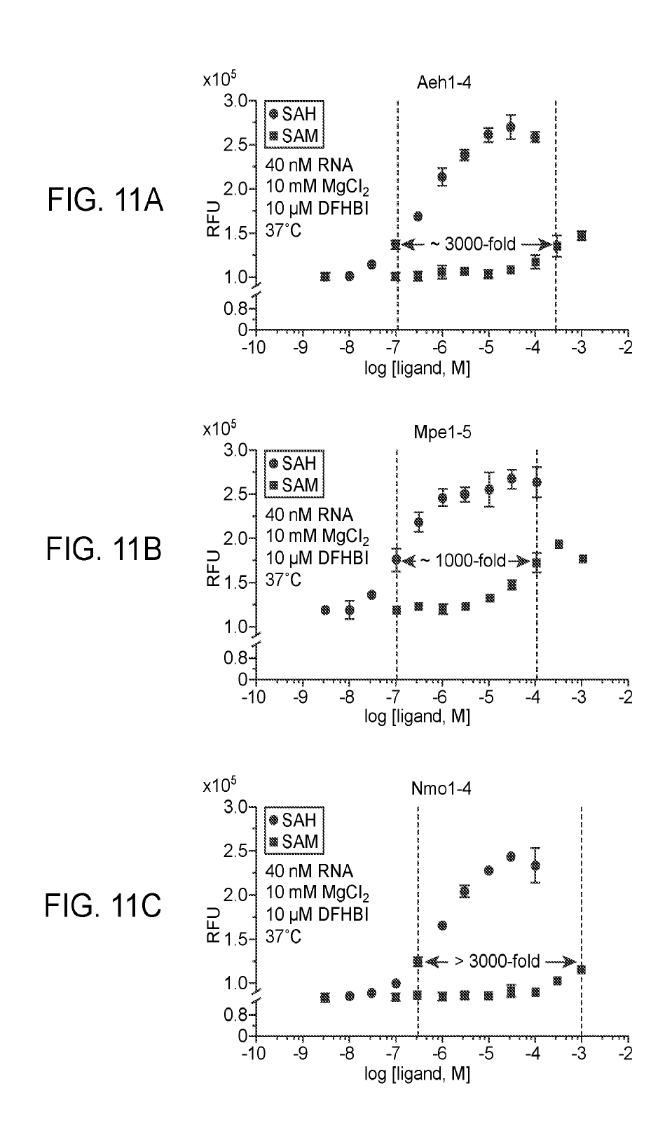

In some instances, the subject biosensor activates fluorescence of the bound signaling chromophore when bound to SAH with a response that is at least 10 fold more sensitive to SAH than SAM, such as at least 30 fold more sensitive, at least 100 fold more sensitive, at least 300-fold more sensitive, at least 1000-fold more sensitive, at least 2000-fold more sensitive, at least 3000-fold more sensitive, or even more sensitive to SAH than SAM, e.g., as measured by a dose response curse (see, e.g., FIG. 11, panels A-C). Based on the response of the biosensor, a level of SAH in the sample may be determined. In some cases, the determined level of SAH is independent of the level of SAM. MTases of interest convert SAM to SAH, and as such, the level of SAH in the sample is dependent, at least in part, on the MTase activity in the cell. In certain embodiments, the subject method further includes determining a methyltransferase activity of the sample based on the determined level of SAH.

Any convenient protocol for contacting the sample with the biosensor may be employed. The particular protocol that is employed may vary, e.g., depending on whether the sample is in vitro or in vivo. For in vitro protocols, contact of the sample with the biosensor may be achieved using any convenient protocol. In some instances, the sample includes cells that are maintained in a suitable culture medium, and the biosensor is introduced into the culture medium. In certain in vitro protocols, no cells are present and the biosensor is simply contacted with other components (e.g., proteins, etc.) of the desired protocol in a convenient container, e.g., vial. For in vivo protocols, any convenient administration protocol may be employed. Depending upon the binding affinity of the biosensor, the response desired, the manner of administration, e.g. intravenous, subcutaneous, intraperitoneal, oral, intramuscular, etc., the half-life, the number of cells present, various protocols may be employed.

The term “sample” as used herein relates to a material or mixture of materials, in some cases, in fluid form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). The term “sample” may also refer to a “biological sample”. As used herein, the term “a biological sample” refers to a whole organism or a subset of its tissues, cells or component parts (e.g. body fluids, including, but not limited to, blood, mucus, lymphatic fluid, synovial fluid, cerebrospinal fluid, saliva, amniotic fluid, amniotic cord blood, urine, vaginal fluid and semen). A “biological sample” can also refer to a homogenate, lysate or extract prepared from a whole organism or a subset of its tissues, cells or component parts, or a fraction or portion thereof, including but not limited to, plasma, serum, spinal fluid, lymph fluid, the external sections of the skin, respiratory, intestinal, and genitourinary tracts, tears, saliva, milk, blood cells, tumors and organs. In certain embodiments, the sample has been removed from an animal or plant. Biological samples may include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi.

Components in a sample are termed “analytes” herein. In many embodiments, the sample is a complex sample containing at least about 10², 5×10², 10³, 5×10³, 10⁴, 5×10⁴, 10⁵, 5×10⁵, 10⁶, 5×10⁶, 10⁷, 5×10⁷, 10⁸, 10⁹, 10¹⁰, 10¹¹, 10¹² or more species of analyte.

The subject methods may further include evaluating the sample for appearance of a detectable signal. Evaluation of the sample may be performed using any convenient method, and at any convenient time before, during or after application of a biosensor to the sample. Evaluation of the sample may be performed continuously, or by sampling at one or more time points during the subject method. Aspects of the subject methods include detecting fluorescence from the biosensor thereby determining the level of SAH in the sample. Detecting fluorescence may include exciting the biosensor with one or more lasers at an interrogation point of the sample, and subsequently detecting fluorescence emission from the signaling chromophore using one or more optical detectors.

Also provided by the present disclosure is a method for determining level of methyltransferase activity in a cell. Aspects of the method include contacting the cell with a single stranded nucleic acid (e.g., as described herein) and a signaling chromophore (e.g., as described herein) to produce a SAH biosensor in situ. In certain instances, contacting the cell with the subject nucleic acid includes expressing a construct encoding the subject nucleic acid (e.g., as described herein). In some instances, contacting the cell with the subject nucleic acid includes introducing a composition including the subject nucleic acid to the cell.

In some instances, the method is a method including detecting fluorescence from the signaling chromophore (e.g., a DFHBI fluorophore) of the SAH biosensor thereby determining the level of an enzyme activity in the sample, where the enzyme either utilizes SAH as a substrate or co-substrate (e.g., methylthioadenosine nucleosidase MTAN) or produces SAH as a product (SAM-dependent methyltransferase). In certain cases, the sample is a cellular sample.

Aspects of the method include detecting fluorescence from the signaling chromophore (e.g., a DFHBI fluorophore) of the SAH biosensor thereby determining the level of methyltransferase activity in the cell. As used herein, the terms “determining,” “measuring,” and “assessing,” and “assaying” are used interchangeably and include both quantitative and qualitative determinations. In certain instances, the methyltransferase activity is assessed by detecting the production of SAH in the cell.

Cells of interest which may be targeted for assessing a methyltransferase activity in the subject methods include, but are not limited to, stem cells, T cells, dendritic cells, B Cells, granulocytes, leukemia cells, lymphoma cells, virus cells (e.g., HIV cells) NK cells, macrophages, monocytes, fibroblasts, epithelial cells, endothelial cells, and erythroid cells. Target cells of interest include cells that contain, or are suspected of containing a SAM-dependent methyltransferase. In some embodiments, the target cell is selected from a virus-infected cell, a regulatory T (Treg) cell, an antigen-specific T -cell, a tumor cell, or a hematopoietic progenitor cell (CD34⁺ cell).

Any convenient SAM-dependent methyltransferases (MTases) may be targeted for assay according to the subject methods. In some embodiments, the MTase is a class I or class V MTase, such as one of those described by Struck et al., ChemBioChem, Volume 13, Issue 18, pages 2642-2655, Dec. 21, 2012. In some cases, the MTase is a protein methyltransferase, see e.g., Richon, V. M. et al. Chemogenetic analysis of human protein methyltransferases, Chem. Biol. Drug Des. 78, 199-210 (2011). In certain instances, the MTase is a histone lysine methyltransferase (HKMT), see e.g., Copeland et al., Nature Rev. Drug Discov. 8, 724-732 (2009).

In certain embodiments, the MTase is SETT. In certain embodiments, the MTase is M.SssI. In certain embodiments, the MTase is a protein lysine MTase SET7/9.

Methyltransferases of interest include, but are not limited to, 5-adenosyl-L-methionine (SAM)-dependent O-methyltransferases (OMT) described by Decker, H. et al. J. Bacteriol. (1993) 175:3876-3886) which are found in polyketide synthase (PKS) gene clusters. Plant O-methyltransferases of interest include, but are not limited to, IOMT from Medicago sativa (alfalfa; AAC49927), ChOMT from Medicago sativa (alfalfa; AAB48059), caffeic acid OMT from Medicago sativa (alfalfa; AAB46623), scoulerine OMT from Coptis japonica (goldenthread; BAA06192), isoeugenol OMT from Clarkia breweri (fairy fans; AAC01533), hydroxymaakiain OMT from Pisum sativa (pea; AAC49856), diphenol OMT from Capsicum annum (hot pepper; AAC17455), catechol OMT from Nicotiana tabacum (tobacco; CAA52461), and flavonoid OMT from Hordeum vulgare (barley; CAA54616).

The subject methods may be performed using any convenient assay formats. In some cases, the method is performed using a flow cytometer. In some instances, the method further includes flow cytometrically analyzing the target cell. Detecting the cell in a flow cytometer may include exciting a biosensor with one or more lasers at an interrogation point of the flow cytometer, and subsequently detecting fluorescence emission from the signaling chromophore using one or more optical detectors. In some embodiments, the methods further include counting and/or sorting the target cells according to the biosensor fluorescence.

### Screening Assays

In some cases, a subject method further comprises monitoring fluorescence of the signaling chromophore upon application of a stimulus to the cell. Any convenient stimuli may be applied to the cells depending on the application of interest, e.g., including research application involving the investigation of an MTase of interest and drug discovery applications involving the identification of an agent that binds to an MTase of interest. In some cases, application of a stimulus to the cell refers to contacting the cell with an agent of interest.

Aspects of the present disclosure also include screening assays configured to identify agents that find use in modulating the activity of an MTase of interest, e.g., as reviewed above. Screening assays of interest include methods of assessing whether a test compound modulates the activity of an MTase of interest. By assessing is meant at least predicting that a given test compound will have a desirable activity, such that further testing of the compound in additional assays, such as animal model and/or clinical assays, is desired. The specific detection of SAH produced by the action of SAM-dependent MTases provides for a sensitive assay of MTAse activity. A subject biosensor may be introduced to a sample in vitro, and the behavior of an MTase of interest in the presence of the test compound may be assessed.

Drug screening may be performed using an in vitro model, a genetically altered cell or animal (e.g., non-human animal), or purified MTase of interest. One can identify ligands or substrates that compete with, modulate or mimic the action of a SAM-dependent MTase. Drug screening can identify agents that modulate MTase activity, either as an antagonist or as an agonist. A wide variety of assays may be adapted for use in conjunction with the subject methods, including labeled in vitro protein-protein binding assays, electrophoretic mobility shift assays, immunoassays for protein binding, and the like.

The term “agent” as used herein describes any molecule, e.g., protein or pharmaceutical, with the capability of modulating an MTase of interest. In some cases, a plurality of assay mixtures are run in parallel with different agent concentrations to obtain a differential response to the various concentrations. In some cases one of these concentrations serves as a negative control, i.e., at zero concentration or below the level of detection.

Candidate agents encompass numerous chemical classes, such as organic molecules, e.g., small organic compounds having a molecular weight of more than 50 and less than about 2,500 daltons. Candidate agents may include functional groups necessary for structural interaction with a target enzyme, particularly hydrogen bonding, and in some cases include at least an amine, carbonyl, hydroxyl or carboxyl group, in some cases at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

### Kits

Aspects of the present disclosure include kits, where the kits include one or more components employed in the biosensors and methods of inventions, e.g., nucleic acids, constructs, host cell, as described herein. In some embodiments, the kit includes a single stranded nucleic acid or a nucleic acid construct encoding the single stranded nucleic acid (e.g., as described herein). The subject kit may further include one or more components selected from a DFHBI fluorophore, SAH, SAM, a promoter, a cell, a cloning vector and an expression cassette. Any of the components described herein may be provided in the subject kit, e.g., cells comprising biosensor systems, biosensors, nucleic acids, constructs (e.g., vectors) encoding for components of the biosensor, e.g., aptamer domains, genomic constructs, components suitable for use in expression systems (e.g., cells, cloning vectors, multiple cloning sites (MSC), bi-directional promoters, an internal ribosome entry site (IRES), etc.), etc. A variety of components suitable for use in making and using constructs, cloning vectors and expression systems may find use in the subject kits. Kits may also include tubes, buffers, etc., and instructions for use. The various reagent components of the kits may be present in separate containers, or some or all of them may be pre-combined into a reagent mixture in a single container, as desired.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), Hard Drive etc., on which the information has been recorded. Yet another form of these instructions that may be present is a website address which may be used via the internet to access the information at a removed site.

### Utility

The biosensors, nucleic acids, constructs and methods of the present disclosure, e.g., as described herein, find use in a variety of different applications where it is desirable to detect SAH or investigate a SAM-dependent methyltransferase of interest. Applications of interest include, but are not limited to, research applications and diagnostic applications. The present disclosure also finds use in applications where biological samples may be assessed for SAM-dependent methyltransferase activity for research, laboratory testing or for use in therapy.

The subject biosensors and methods find use in a variety of research applications. The subject biosensors and methods may be used as a research or diagnosis tool for diseases such as cancer, HIV infection, and diabetes.

The following examples are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Design of a Riboswitch-Based Fluorescent Biosensors for SAH

Fluorescent biosensors were developed that provide direct detection of SAH. In order to incorporate a riboswitch aptamer with a 3′ terminal pseudoknot, an inverted fusion design is demonstrated to a circular permutant of the Spinach2 aptamer, cpSpinach2 (FIG. 1). The cpSpinach2 aptamer functions as a fluorophore-binding RNA with similar fluorescence turn-on properties as Spinach2. Using this design strategy, a phylogenetic screen of riboswitch sequences identified fluorescent biosensors with selectivity (e.g., at least 100-fold selectivity) and affinities for SAH ranging from 0.075-1.3 μM. Using one of these SAH biosensors, a fluorescence-based assay was developed to measure the activity of DNA CpG MTase M.Sssl and protein lysine MTase SET7/9. The reliability of the assay was demonstrated in a high-throughput format for substrate and inhibitor screens.

Other strategies for construction of allosteric RNA-based fluorescent biosensors have taken advantage of the formation of a paired stem (P1′) by the 5′ and 3′ ends of the ligand-sensing aptamer domains (see Paige et al., Science 2012, 335, 1194; Kellenberger et al., J. Am. Chem. Soc. 2013, 135, 4906-4909; and Kellenberger et al., Proc. Natl. Acad. Sci. 2015, 112, 5383-5388). However, structural studies revealed that the SAH riboswitch aptamer harbors a 3′ pseudoknot that forms part of the ligand-binding pocket (Edwards et al., RNA 2010, 16, 2144-55), which spaces the 5′ and 3′ ends considerably apart. Thus, in order to design an SAH biosensor, an alternate design was developed in which an internal paired stem (P2′) of the riboswitch aptamer is used as the transducer stem to connect to the fluorophore-binding domain, the Spinach2 aptamer. However, fusion to Spinach2 would lead to a bipartite biosensor composed of two single-stranded RNAs. It was reasoned that if a circular permutant of the Spinach2 aptamer was functional, an SAH biosensor could be generated via an inverted design (FIG. 1). As shown, the cpSpinach2 is inserted internally in place of the natural P2 loop of the riboswitch aptamer, with a GCAA loop sequence added to link the original 5′ and 3′ ends (FIG. 2).

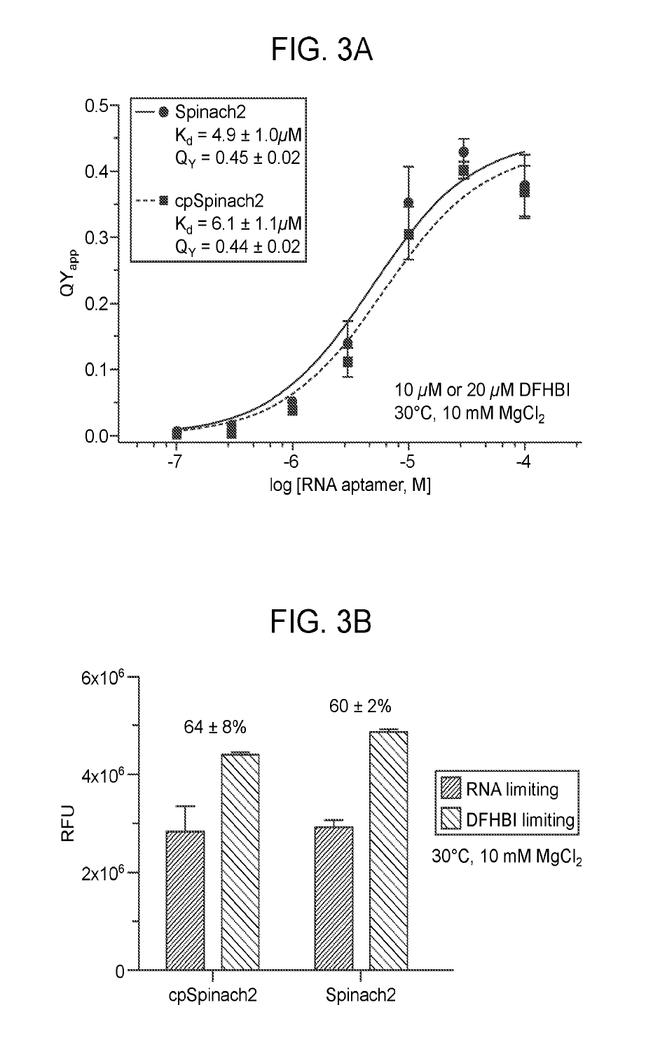

The dye binding and fluorescence activation of cpSpinach2 was examined in comparison to Spinach2 in vitro. Following a previously reported method (see Babendure et al., J. Am. Chem. Soc. 2003, 125, 14716-7.), the quantum yield (QY) and apparent dissociation constants (K_{d}) of the dye-aptamer complexes were derived (FIG. 3, panel A), which showed that there was no significant difference in performance between the two aptamers under these conditions. A comparison to literature-reported values of DFHBI-Spinach2 complexes is given in Table 1.

TABLE 1
Comparison of photophysical and binding properties of DFHBI-RNA aptamer complexes.

| | Excitation | Emission | Extinction | Fluorescence | | |
|---|---|---|---|---|---|---|
| | Maximum | maximum | coefficient | quantum | K_{d} | Tₘ |
| | (nm) | (nm) | (M⁻¹⁻ cm⁻¹) | yield | (μM) | (° C.) |
| DFHBI | 423 | 489 | 30100 | 0.0007 | — | — |
| (Nawrocki et | | | | | | |
| al., Nucleic | | | | | | |
| Acids Research | | | | | | |
| 2015, 43, D130- | | | | | | |
| 137) | | | | | | |
| DFHBI | 428 ± 1 | 502 ± 1 | 30018 | 0.00072 ± 0.00013 | — | — |
| DFHBI- | 447 | 501 | 22000 | 0.72 | 0.530 | 38 ± 0.4 (1 mM Mg²⁺) |
| Spinach2 | | | | | | |
| (Nawrocki et | | | | | | |
| al.) | | | | | | |
| DFHBI- | 450 ± 2 | 506 ± 1 | — | 0.45 ± 0.02 | 2.1 ± 0.8 (30° C.)** | 37 ± 0.3 (1 mM Mg²⁺) |
| Spinach2 | | | | | | |
| | | | | | 4.9 ± 1.0 (30° C.)* | 40 ± 0.4 (3 mM Mg²⁺) |
| | | | | | 8.6 ± 2.2 (37° C.)** | 40 ± 0.3 (10 mM Mg²⁺) |
| DFHBI- | 450 ± 2 | 506 ± 1 | — | 0.44 ± 0.02 | 2.0 ± 0.5 (30° C.)** | 36.5 ± 0.3 (1 mM Mg²⁺) |
| cpSpinach2 | | | | | | |
| | | | | | 6.1 ± 1.1 (30° C.)* | 39.5 ± 0.3 (3 mM Mg²⁺) |
| | | | | | 5.4 ± 1.0 (37° C.)** | 40 ± 0.3 (10 mM Mg²⁺) |

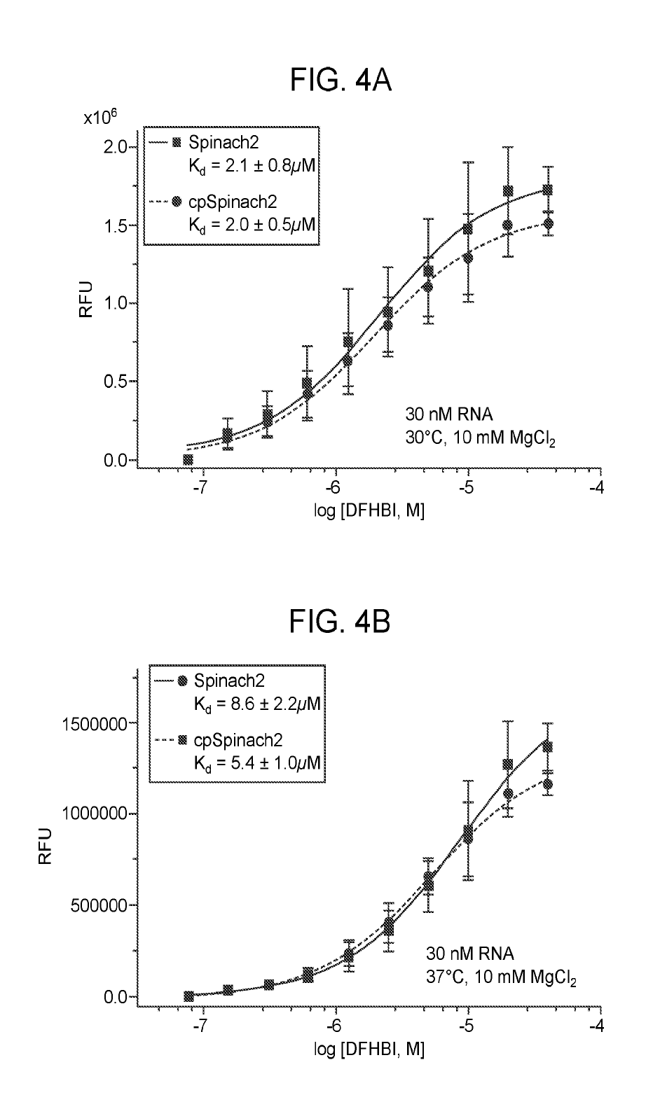

*From measurement of quantum yield by titrating varied concentrations of RNA aptamer to a fixed concentration of DFHBI; for more details, see FIG. 3A.
**From measurement of fluorescence by titrating varied concentrations of DFHBI to fixed concentration of RNA aptamer; for more details, see FIG. 4A, FIG. 4B.

One caveat to the apparent K_{d} values determined above is that it was determined that only a fraction of total RNA is functionally folded (˜60% at 30° C., FIG. 3, panel B). Taking this into account, the corrected K_{d} values are closer to those determined by measuring fluorescence increase with titration of DFHBI to a solution containing the RNA aptamer at limiting concentrations (FIG. 4, panel A). It also should be noted that the K_{d} values are temperature sensitive, since a decrease in DFHBI binding affinity was observed for both aptamers when the assay was performed at higher temperature (37° C.) (FIG. 4, panel B).

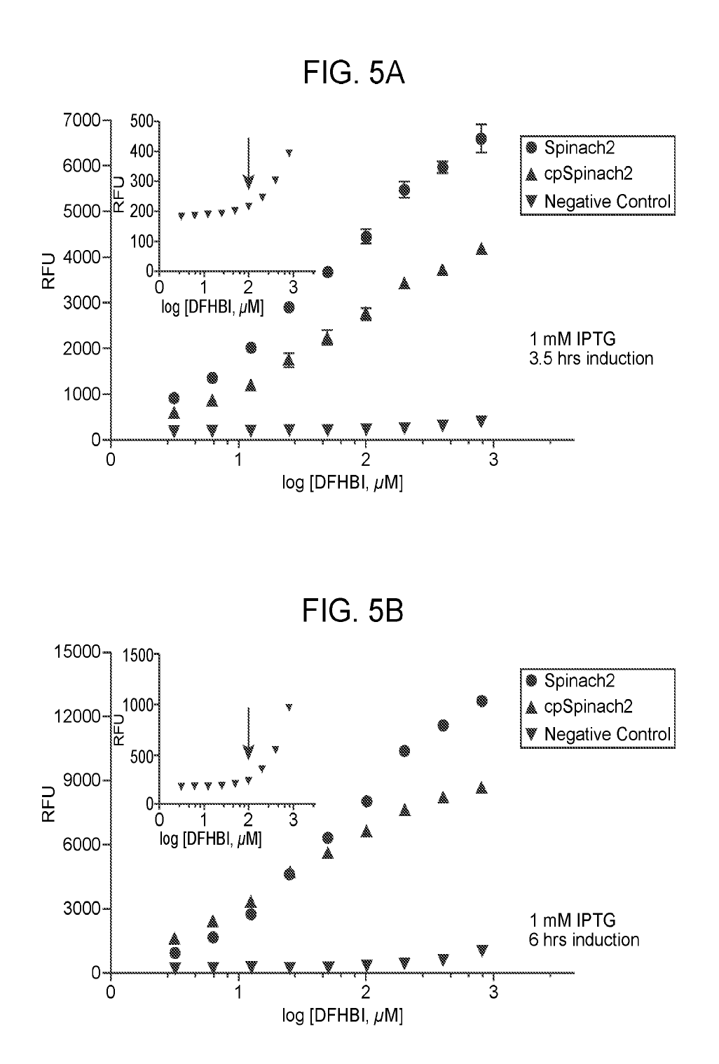

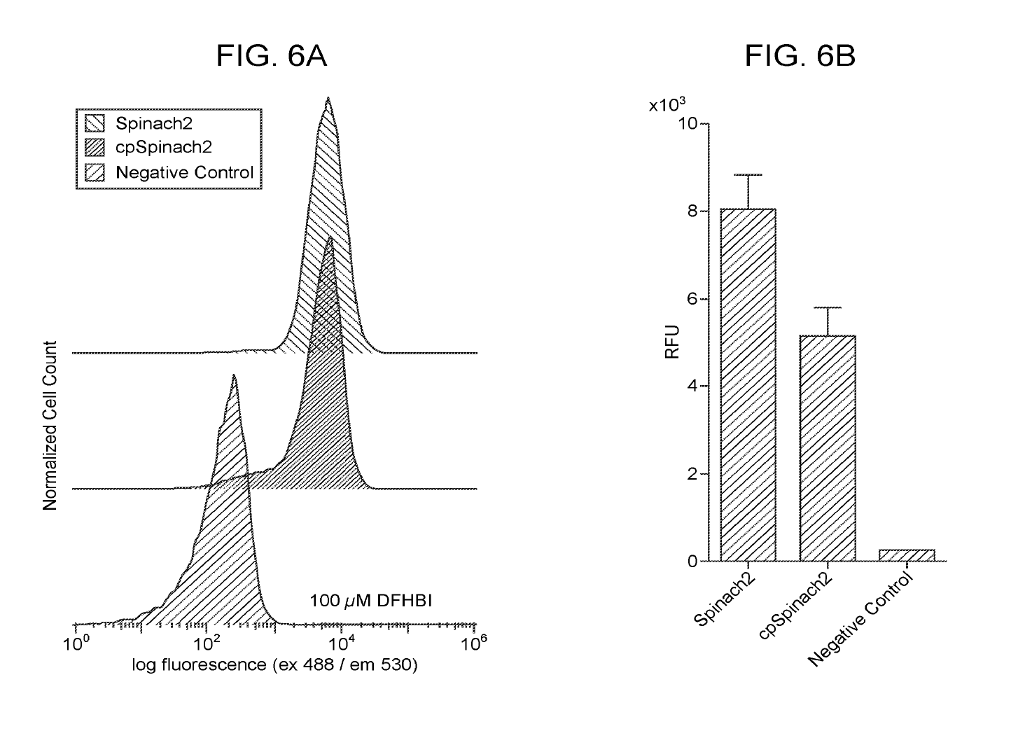

To compare the fluorescence of Spinach2 and cpSpinach2 in live cells, pET31b plasmids encoding the RNA aptamers in a tRNA scaffold were transformed into E. coli BL21* cells. Following IPTG induction of transcription, cells were incubated with DFHBI and fluorescence was quantitated by flow cytometry. The mean cellular fluorescence was measured as a function of increasing DFHBI concentration in the media, in order to determine the optimal conditions for aptamer fluorescence. Unexpectedly, full saturation of fluorescence was not observed, even when E. coli cells were incubated in media containing up to 800 μM DFHBI (FIG. 5). However, the non-specific fluorescence signal increased when DFHBI concentration exceeded 100 μM. Thus, to balance higher fluorescence activation and lower non-specific background, a concentration of 100 μM was adopted for further experiments. Under these conditions, cellular fluorescence activation for Spinach2 and cpSpinach2 was on average 34 and 22-fold over background, respectively (FIG. 6). Thus, cpSpinach2 is a functional alternative to Spinach2. Although it currently performs slightly poorer in terms of fluorescence activation in live cells, improvements are likely possible through directed mutagenesis for higher thermal stability, improved folding efficiency and less Mg²⁺ dependency. Interestingly, this result also suggests that fully circular Spinach2 aptamers or Spinach-based biosensors should also retain activity.

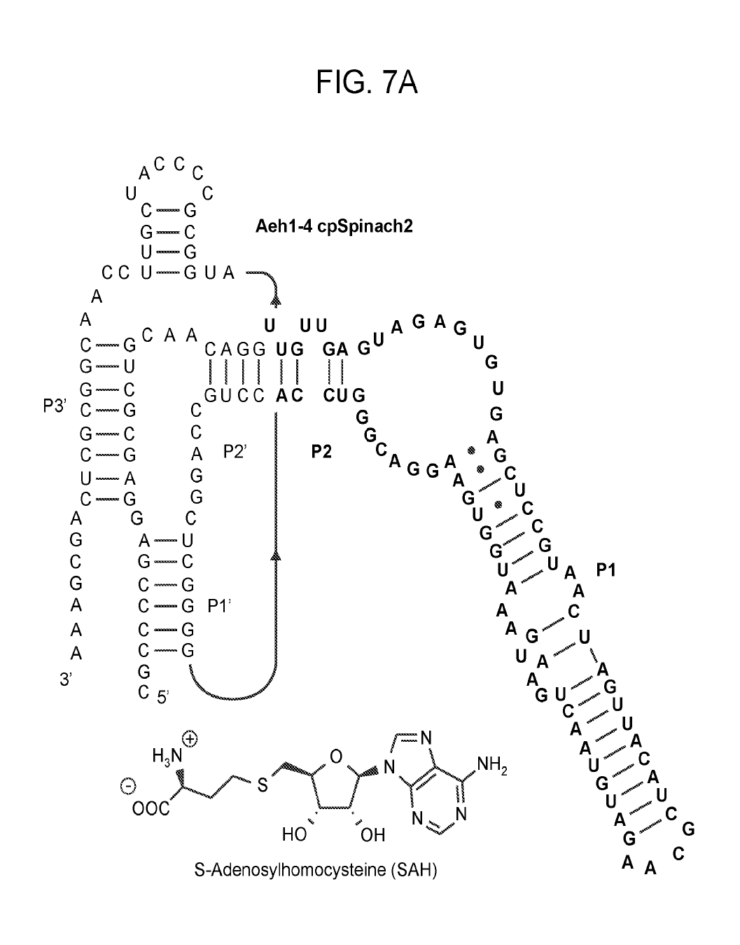

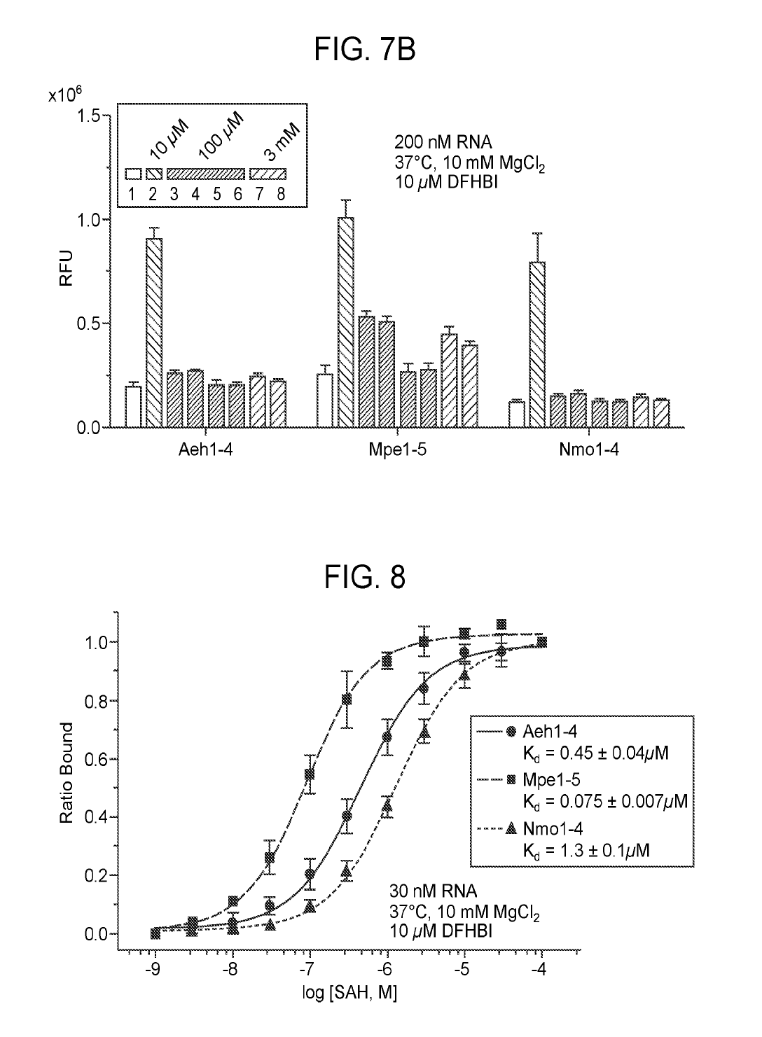

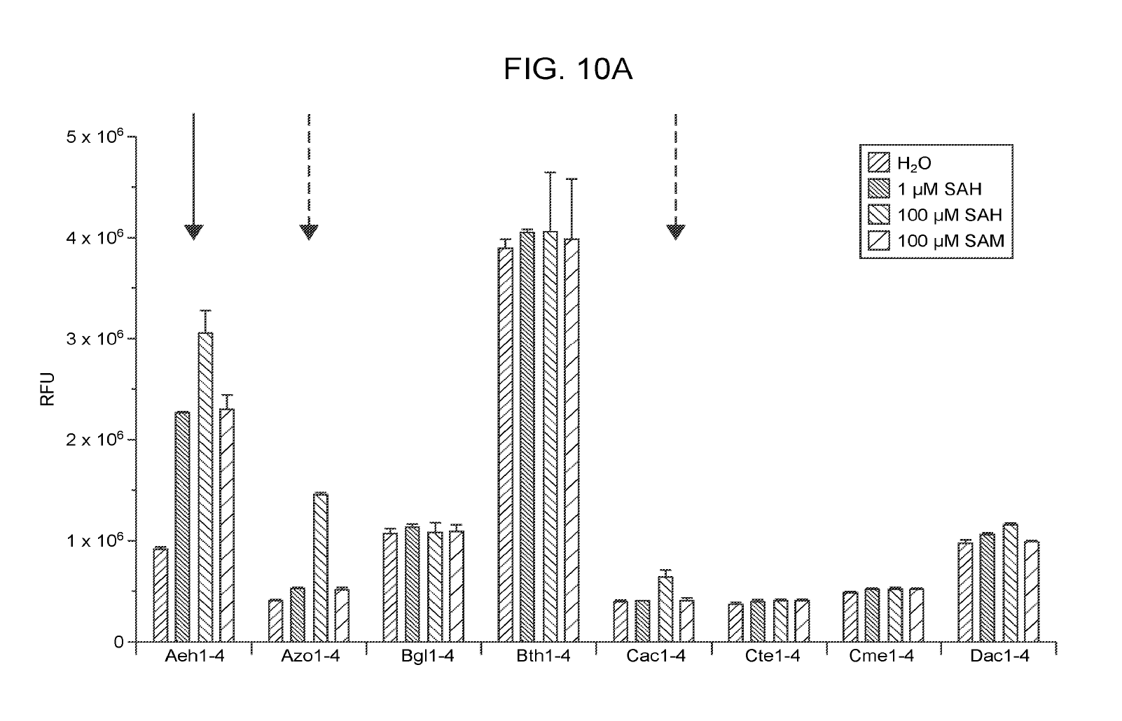

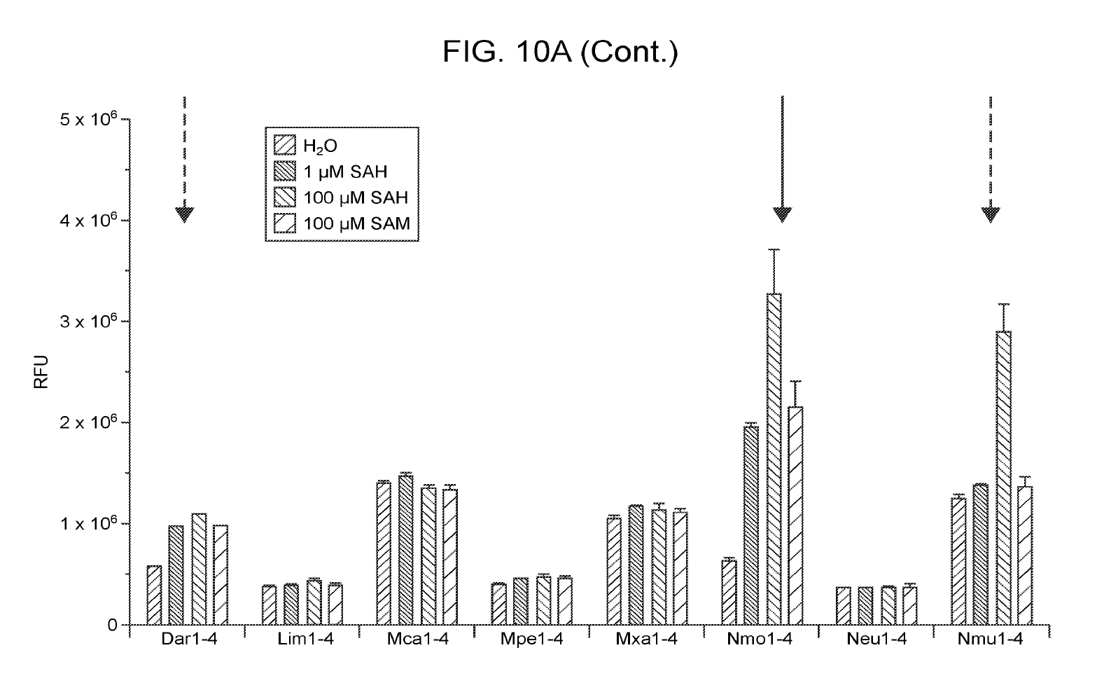

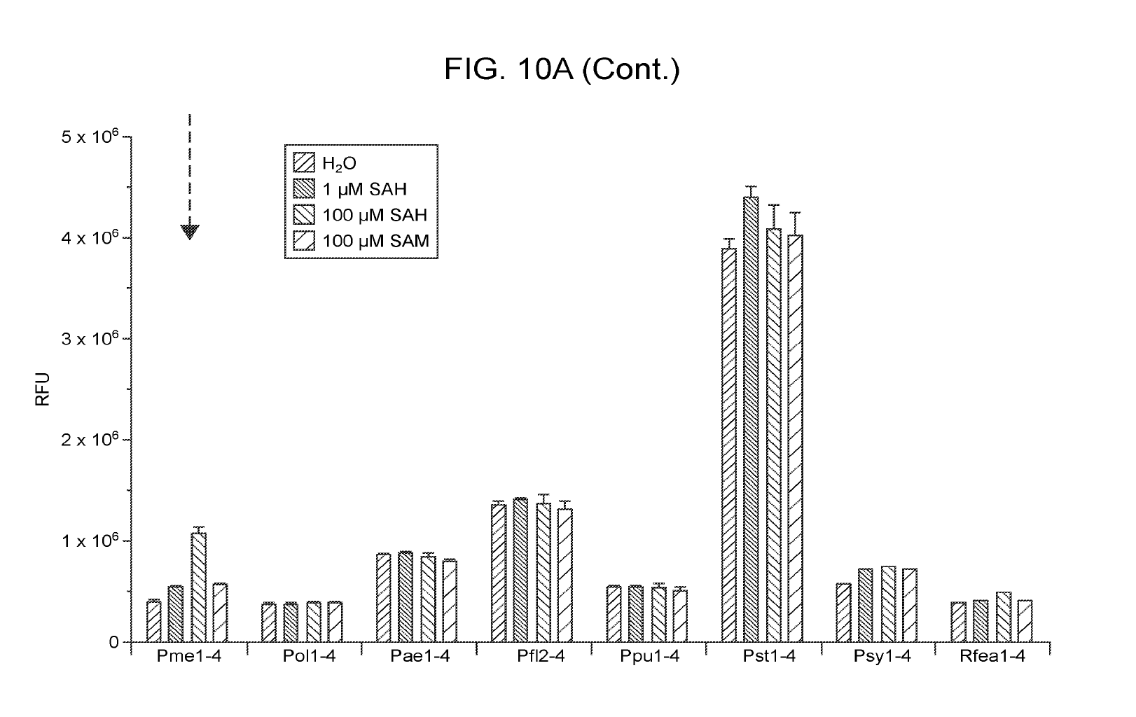

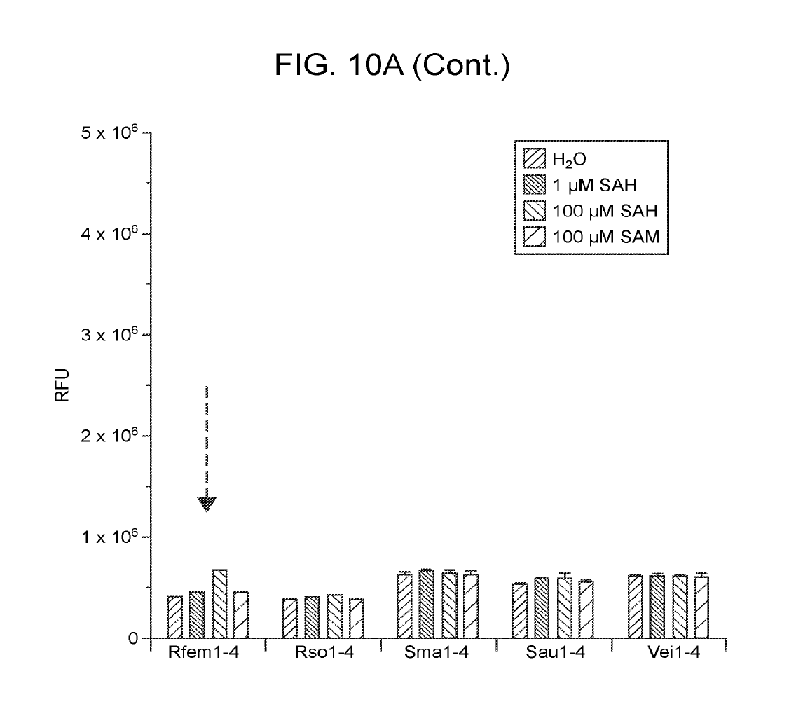

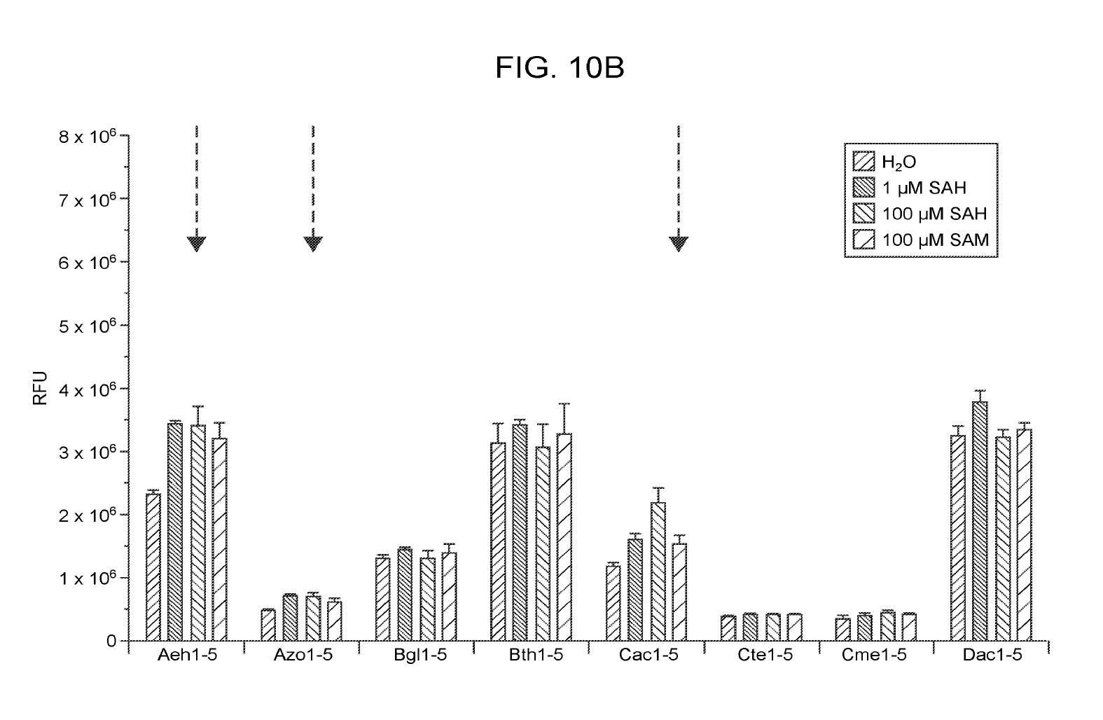

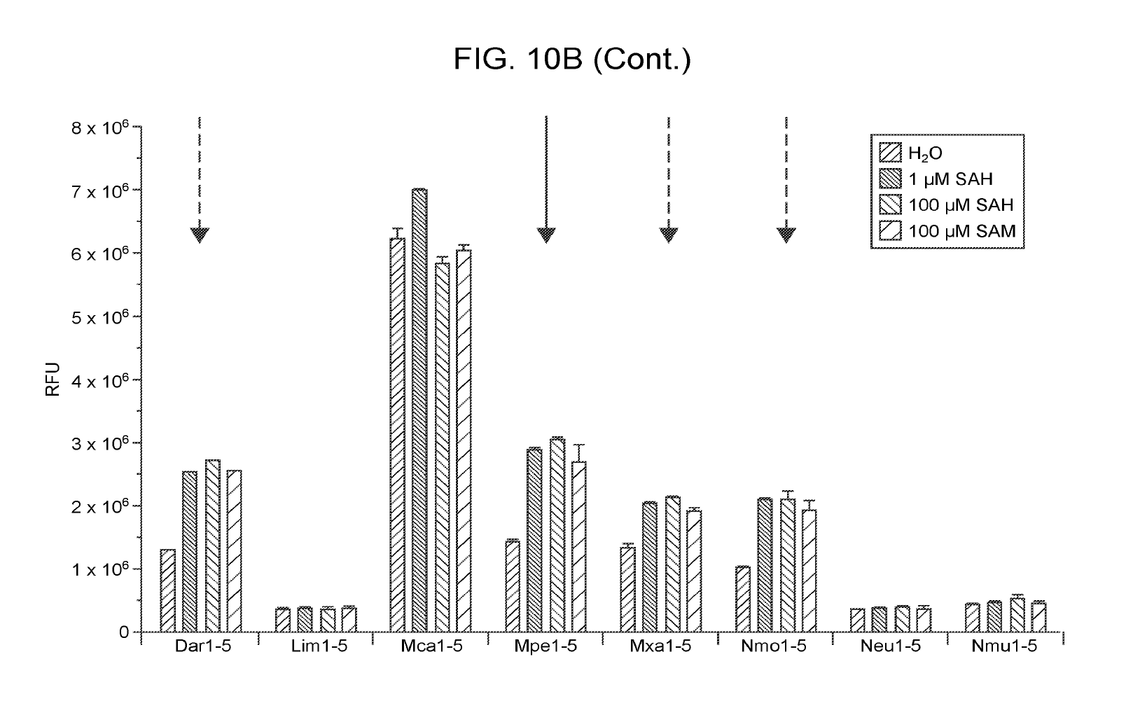

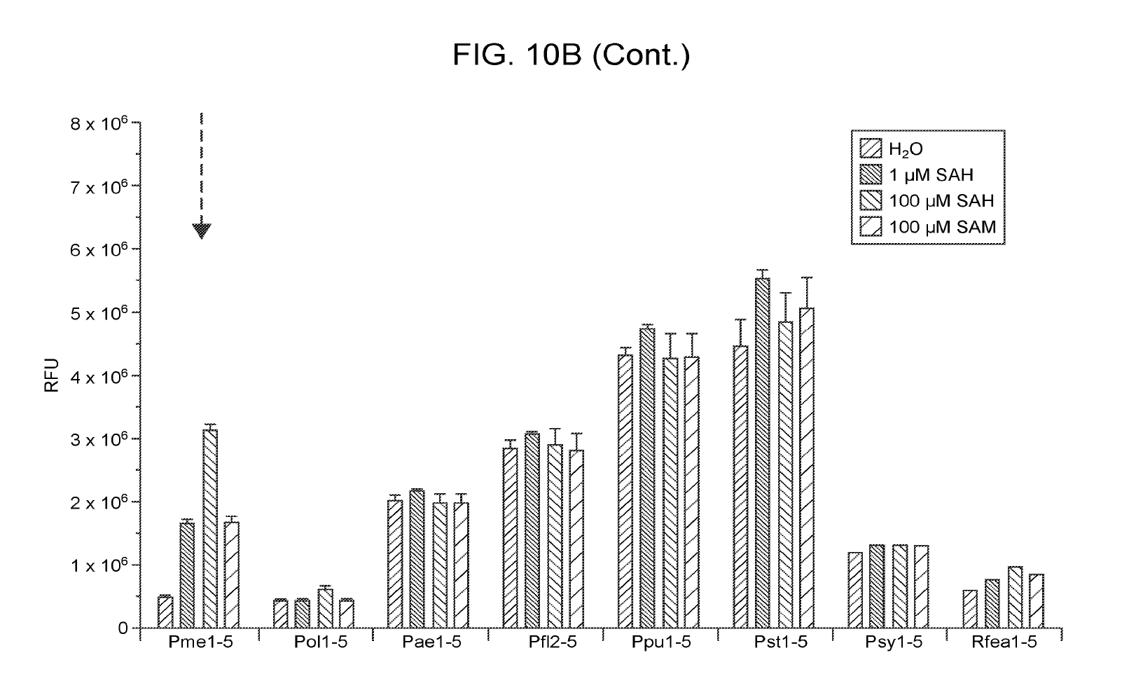

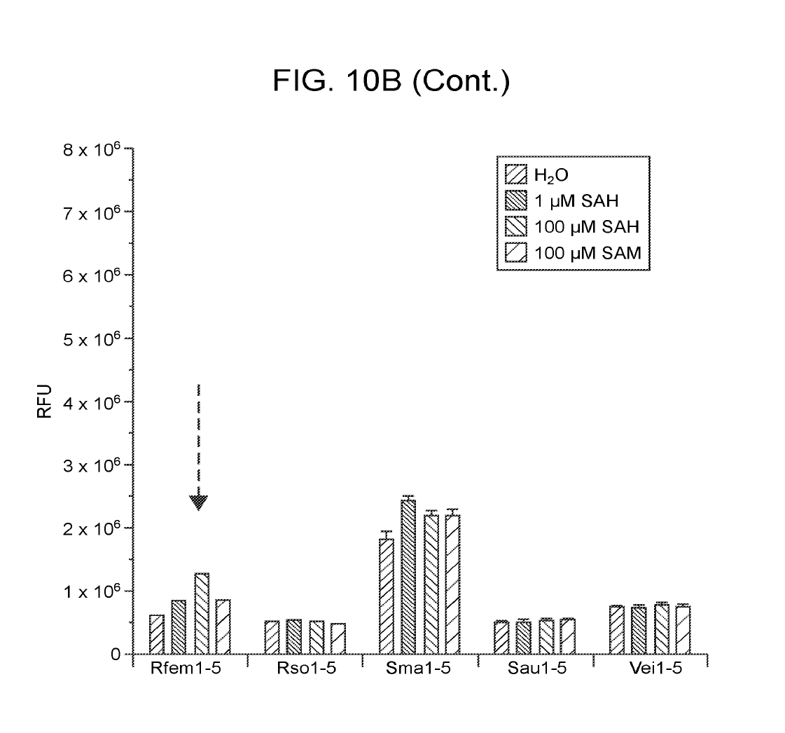

Sampling the phylogenetic diversity of riboswitch sequences can lead to improved biosensors. 29 representatives were selected from among the seed sequences of the SAH riboswitch family deposited in Rfam (Nawrocki et al., Nucleic Acids Res. 2014, 43, D130-D137) and the P2′ loops were replaced with cpSpinach2 (FIG. 7, panel A, FIG. 9A-9B). The natural P2′ stems are longer than 4 base pairs, but were truncated to 4 or 5 base pairs. In this way, 58 biosensor candidates were constructed and screened for fluorescence activation upon addition of SAH or SAM (FIG. 10). In the initial screen, 17 of the putative biosensors showed selective fluorescence response to SAH.

Further analysis was performed on three hits that showed more than 2-fold fluorescence activation in the screen (solid arrows, FIG. 10). These biosensors incorporated SAH riboswitches from Alkalilimnicola ehrl, Methylibium trolei, and Nitrococcus mobilis and were called Aeh1-4, Mpe1-5, and Nmo1-4, respectively. The effect of Mg²⁺ and DFHBI concentrations on fluorescence turn-on was investigated and found to have differential effects that depend on the riboswitch sequence (results not shown).

Magnesium concentrations in fluorescent assays. Magnesium often is a critical cofactor for RNA folding. Folding of the Spinach aptamer has been described as highly magnesium-dependent, (see Filonov et al., Journal of the American Chemical Society 2014, 136, 16299-16308) and magnesium appears to promote folding and stabilize the binding pocket of the SAH riboswitch (Edwards et al., RNA 2010, 16, 2144-2155). Magnesium concentration was investigated in an effort to improve the fluorescence turn-on of the biosensors (around 5-, 4-, 9-fold for Aeh1-4, Mpe1-5 and Nmo1-4, respectively, at 37° C., 10 mM Mg²⁺). Interestingly, only Aeh1-4 exhibited marked improvement in fluorescence turn-on with increasing magnesium from 1 to 20 mM. For the other two biosensors, increases in fluorescence activation with SAH was counteracted by increases in fluorescence background without SAH, so fold turn-on remained relatively unchanged.

However, the biosensors are uniformly selective for SAH over related metabolites, though a slight response to SAM was observed, especially with Mpe1-5 with 10 μM of SAM (FIG. 7, panel B). Comparison of fluorescence responses to SAH and SAM as a function of ligand concentrations revealed that in fact, all three of the biosensors are ˜1000-fold selective for SAH over SAM at 37° C. (FIG. 11, panels A-C). The Mpe1-5 biosensor exhibits the highest affinity for SAH (K_{d} of 75±7 nM at 10 mM Mg²⁺, 37° C., FIG. 8), which explains its responsiveness to mid-micromolar concentrations of SAM. The combined suite of three biosensors have a dynamic range that spans 3 orders of magnitude for detection of SAH, from tens of nanomolar to tens of micromolar.

The affinity for SAH and the selectivity between SAH and SAM are reduced relative to values reported (see Wang et al., Mol. Cell 2008, 29, 691-702) for natural SAH riboswitch aptamers for these biosensors. This may be partly due to differences in conditions for the assays. Also, the specific SAH riboswitch aptamers identified in the biosensor screen had not been previously characterized, nor had the effect of truncations of the P2 stem been evaluated. It should be noted, however, that these first-generation biosensors still exhibit selectivity for SAH over SAM that is comparable to that of commercial antibodies for SAH (180-fold) (see Graves et al., Anal. Biochem. 2008, 373, 296-306) In fact, the selectivity of the subject biosensors is likely higher than ˜100-fold, as the commercial source of SAM utilized guarantees only 80-90% purity due to hydrolysis to SAH. Taken together, these results present riboswitch-based biosensors as a practical alternative to antibodies for selective detection of small molecules like SAH. Advantages for assay development using RNA-based fluorescent biosensors include ease of rational design, synthesis, and testing in vitro.

### Example 2

### Sequence analysis of Biosensors

Based on the results of the phylogenetic screen, sequences of interest were selected that have more than 1.4×fluorescence turn-on between adding H₂O and 100 μM SAH. The screen was performed on 29 riboswitches, with two lengths of stem (4 bp or 5 bp) for biosensors construction. Most of the hits that have more than 1.4×fluorescence turn-on appeared as pairs (both 4 bp and 5 bp). Thus, for the purpose of consensus sequence alignment, length of stem is ignored in the following alignments.

The following sequences are the biosensors hits that meet the aforementioned criteria. Those portions of the sequences that are underlined stand for cpSpinach2 sequences while the portions of the sequences that are in bold and italic stand for the transducer stem between SAH riboswitch and cpSpinach2 aptamer sequences.

>Pme
(SEQ ID NO: 32)
CCGTCCGAGGGGCGCTGCAG [symbol] TTGTTGAGTAGAGTGTGAGCTCCGTAACTAGTTACATCGCAAGAT
GTAACTGAATGAAATGGTGAAGGACGGGTCCA [symbol] TCAGGCTCGGATGGGGCGTTAACCATACGCATC
AACGGCGCCCATTCGCA
>Nmo
(SEQ ID NO: 33)
CTCGCCGAGGAGCGCTGCAA [symbol] TTGTTGAGTAGAGTGTGAGCTCCGTAACTAGTTACATCGCAAGAT
GTAACTGAATGAAATGGTGAAGGACGGGTCCA [symbol] CCAGGCTCGGCGATTATCGGGACCTTTAAACCA
ACGGCGCTCGG
>Aeh
(SEQ ID NO: 34)
CGCCCCGAGGAGCGCTGCAA [symbol] TTGTTGAGTAGAGTGTGAGCTCCGTAACTAGTTACATCGCAAGAT
GTAACTGAATGAAATGGTGAAGGACGGGTCCA [symbol] CCAGGCTCGGGGATGGCGCCCCATCGTTCCAAC
GGCGCTCAGCGAAA
>Azo
(SEQ ID NO: 35)
CCTGCCGAGGAGCGCTGCGA [symbol] ATTGTTGAGTAGAGTGTGAGCTCCGTAACTAGTTACATCGCAAGAT
GTAACTGAATGAAATGGTGAAGGACGGGTCCAC [symbol] CCAGGCTCGGCAACCGAACAACGGCGCTCGCAA
ACC
>Nmu
(SEQ ID NO: 36)
CAGACCGAGGAGCGCTGCAA [symbol] CTTGTTGAGTAGAGTGTGAGCTCCGTAACTAGTTACATCGCAAGAT
GTAACTGAATGAAATGGTGAAGGACGGGTCCAT [symbol] CGAGGCTCGGCAAGGAAGGATCGCTACAACGGC
GCTCGTTCATT
>Dar
(SEQ ID NO: 37)
TGCCGAGGAGCGCTGCGA [symbol] TTGTTGAGTAGAGTGTGAGCTCCGTAACTAGTTACATCGCAAGATGT
AACTGAATGAAATGGTGAAGGACGGGTCCA [symbol] CCAGGCTCGGCAATGATCAACGGCGCTCGCAAACC
>Rfem
(SEQ ID NO: 38)
TCTTCCAAGGAGCGTTGCAG [symbol] TTGTTGAGTAGAGTGTGAGCTCCGTAACTAGTTACATCGCAAGAT
GTAACTGAATGAAATGGTGAAGGACGGGTCCA [symbol] TCAGGCTTGGATGACCCCAACGACGCTCACCTG
AT
>Mpe
(SEQ ID NO: 39)
ACTTTCGAGGAGCGTTGCAA [symbol] TTGTTGAGTAGAGTGTGAGCTCCGTAACTAGTTACATCGCAAGAT
GTAACTGAATGAAATGGTGAAGGACGGGTCCA [symbol] CCAGGCTCGAAAGTCATCCCCCGATGCAACGGC
GCTCACCCGCA
>Cac
(SEQ ID NO: 40)
CAATCCGAGGAGCGCTGCA [symbol] TTGTTGAGTAGAGTGTGAGCTCCGTAACTAGTTACATCGCAAGAT
GTAACTGAATGAAATGGTGAAGGACGGGTCCA [symbol] TCAGGCTCGGATTCCTTAAACGGCGCTCATCCG
AC
>Rfea
(SEQ ID NO: 41)
TTCCGAGGAGCGTTGCA [symbol] TTGTTGAGTAGAGTGTGAGCTCCGTAACTAGTTACATCGCAAGATGTA
ACTGAATGAAATGGTGAAGGACGGGTCCA [symbol] GAGGCTCGGAGCACGCGGCCCTGATGGGGCTGCGAC
AGTCAACGGCGCTCACCCACT
>Mxa
(SEQ ID NO: 42)
GACTTCGAGGAGCGCTGCGA [symbol] TTGTTGAGTAGAGTGTGAGCTCCGTAACTAGTTACATCGCAAGAT
GTAACTGAATGAAATGGTGAAGGACGGGTCCA [symbol] CCAGGCTCGAAGCGGAATCACCCAACGGCGCTC
ACCTGTA
>Pol
(SEQ ID NO: 43)
CTTCCTGAGGAGCGTTGCAAC [symbol] TTGTTGAGTAGAGTGTGAGCTCCGTAACTAGTTACATCGCAAGAT
GTAACTGAATGAAATGGTGAAGGACGGGTCCA [symbol] ACCAGGCTCAGGACTTCATTGCAACCACGCTCAC
CCGCA

It is understood that any of the subject nucleic acids may be a DNA sequence that includes T nucleotides. In some cases, the subject nucleic acid is a RNA sequences wherein U nucleotides are utilized in place of T nucleotides in the sequences set forth herein.

Based on the sequences selected above and the structure of the biosensor, the following biosensor consensus sequence was devised:
NNNNYYRAGGRGCGY [symbol] NNNNNTTGTTGAGTAGAGTGTGAGCTCCGTAACTAGTTACA TCGCAAGATGTAACTGAATGAAATGGTGAAGGACGGGTCCANNNNN [symbol] TYRR(N)ₙ [symbol] [symbol] RCGCYCRNNNNNN (SEQ ID NO:123), where N represents any convenient nucleotide; R represents G or A nucleotides; and Y represents C or T nucleotides. Those nucleotides in bold and italic in SEQ ID NO: 123 represent nucleotides that are located within a close distance from the SAH binding pocket.

In addition, the following biosensor consensus sequence was also devised:
NNNNYC[G]AGG[G]GCGYTGCRRNNNNNTTGTTGAGTAGAGTGTGAGCTCCGTAACTAGTTA CATCGCAAGATGTAACTGAATGAAATGGTGAAGGACGGGTCCANNNNNYYAGGCT[CGG](N)ₙC AACGRCGC[C]CRNNNNNN (SEQ ID NO:124) where N represents any convenient nucleotide; R represents G or A nucleotides; Y represents C or T nucleotides, and those nucleotides in brackets are further specified by comparison to SEQ ID NO:123.

In addition, the following biosensor consensus sequence was also devised:
NNNN[C]CGAGGGGCGYTGCRRNNNNNTTGTTGAGTAGAGTGTGAGCTCCGTAACTAGTTAC ATCGCAAGATGTAACTGAATGAAATGGTGAAGGACGGGTCCANNNNNY[C]AGGCTCGG(N)ₙC AACG[G]CGCCCRNNNNNN (SEQ ID NO:125) where N represents any convenient nucleotide; R represents G or A nucleotides; and Y represents C or T nucleotides and those nucleotides in brackets are further specified by comparison to SEQ ID NO:124.

In addition, the following biosensor consensus sequence was also devised:
NNNNCCGAGGGGCGYTGC[A]RNNNNNTTGTTGAGTAGAGTGTGAGCTCCGTAACTAGTTAC ATCGCAAGATGTAACTGAATGAAATGGTGAAGGACGGGTCCANNNNNYCAGGCTCGG(N)ₙC AACGGCGCCCRNNNNNN (SEQ ID NO:126) where N represents any convenient nucleotide; R represents G or A nucleotides; and Y represents C or T nucleotides and those nucleotides in brackets are further specified by comparison to SEQ ID NO:125.

### Example 3

### High-Throughput MTase Activity Assays

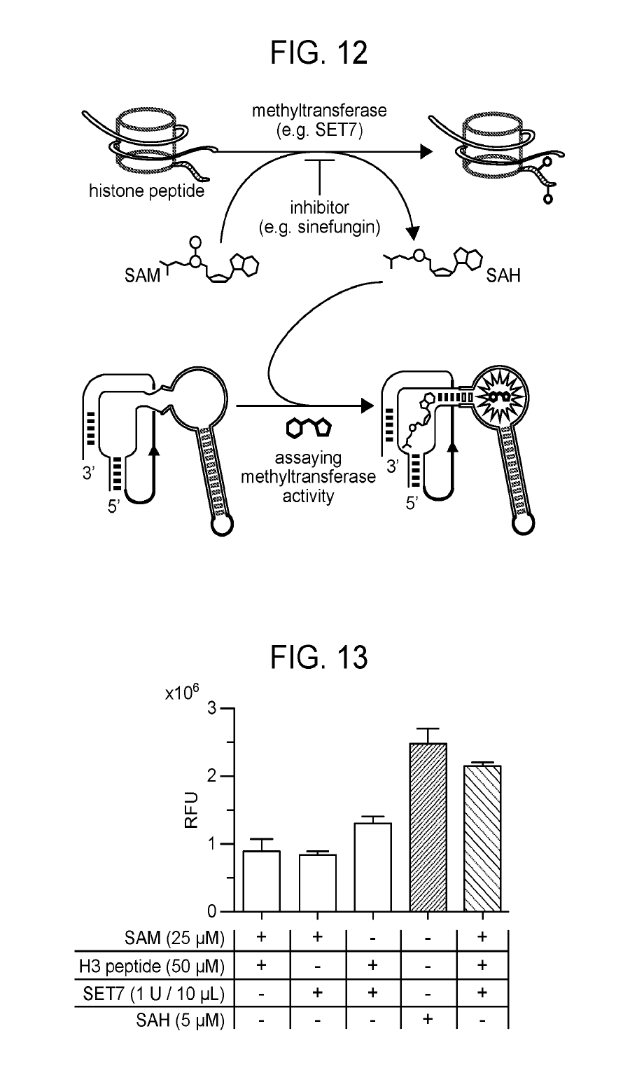

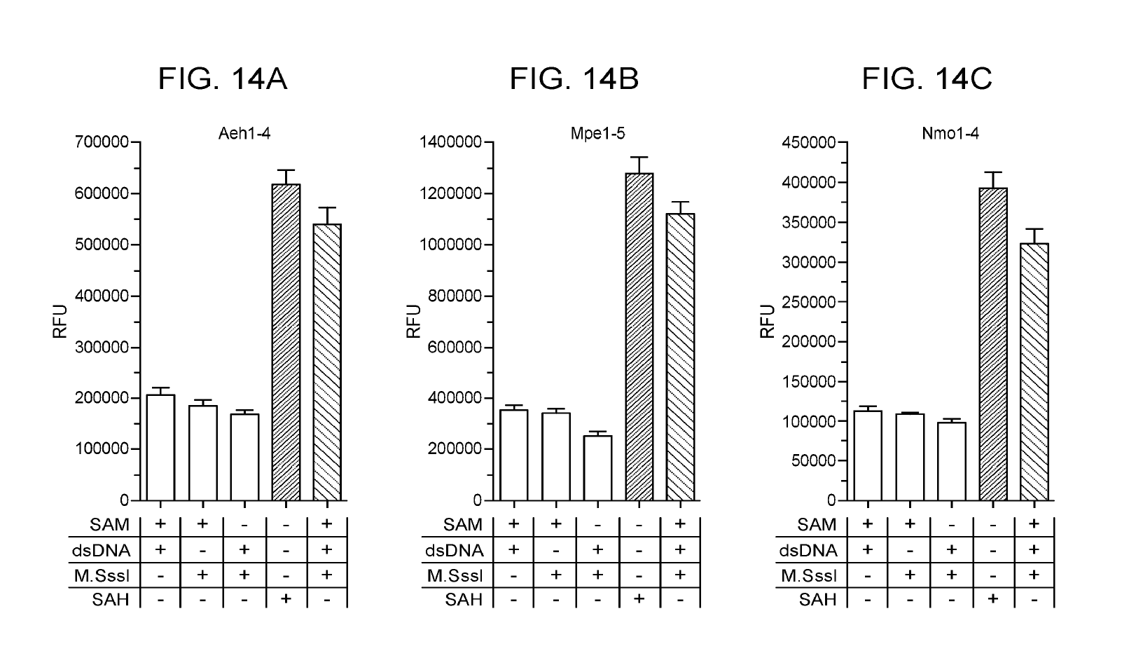

The use of riboswitch-based biosensors was investigated in the development of high-throughput MTase activity assays (FIG. 12). As shown, enzymatic reactions were performed, and then a fixed aliquot of the reaction mixture was diluted into the assay reaction containing the RNA biosensor, DFHBI, and buffer. The bacterial DNA MTase M.SssI possesses robust enzymatic activity and shares structural similarities with human DNA MTase 1 (DNMT1), an important epigenetic regulator and drug target (see Brueckner, et al., Cancer Res. 2005, 65, 6305-6311; and Hupkes et al., J. Biomol. Screen. 2012, 18, 348-355). Using any of the fluorescent biosensors, M.SssI activity was detected as an increase in fluorescence only in the presence of both SAM and dsDNA substrate (FIG. 14). To determine the generality of the strategy, this assay was also tested on a histone protein MTase. Protein lysine MTases are of great interest in epigenetic and pharmaceutical research, but they have lower turnover rates than DNA MTases, which makes them harder to assay. With the exception of DOT1L, all protein lysine MTases harbor a conserved domain (SET) for methylation, therefore SET domain-containing MTase 7/9 (SET7/9) has been established as a model MTase (see Xiao et al., Nature 2003, 421, 652-656; and Luo, ACS Chem. Biol. 2012, 7, 443-63). As shown, the Aeh1-4 biosensor displayed a fluorescence response to SET7/9 activity, although signal-to-noise was lowered by increased background fluorescence in the control without SAM (FIG. 13). Such increased background might be due to incomplete removal of SAM or SAH during the purification of commercial SET7/9 MTase. These results show that MTase activity can be detected by RNA-based biosensors in a simple mix-and-go format with read-out in a fluorescent plate reader.

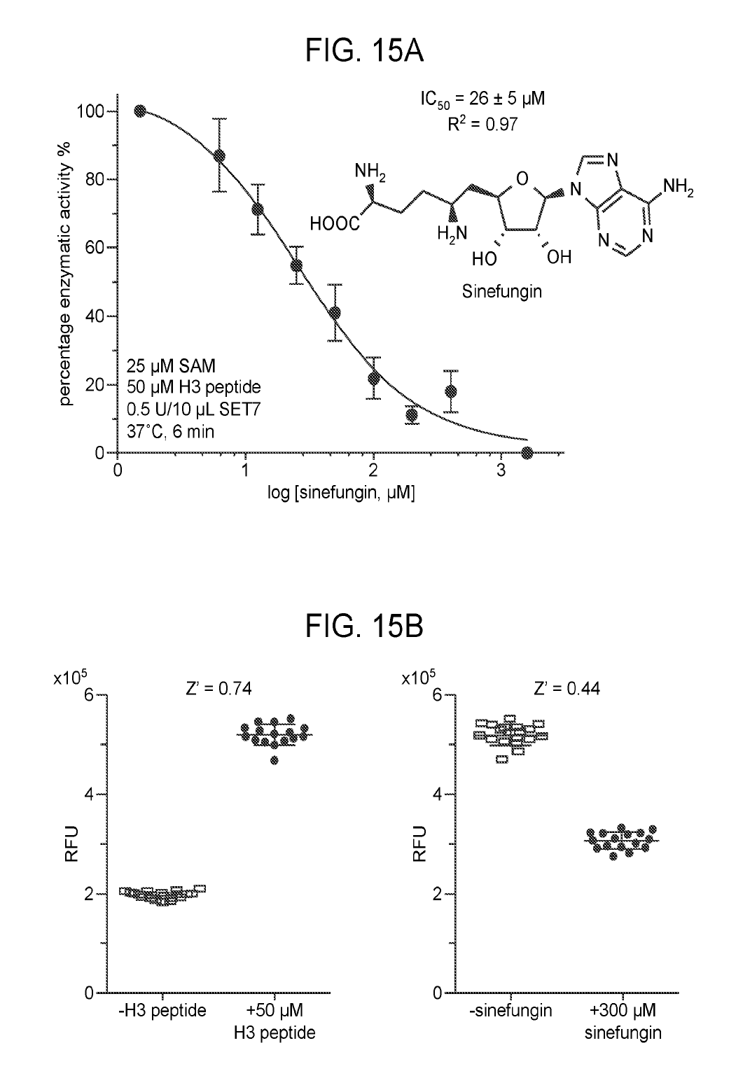

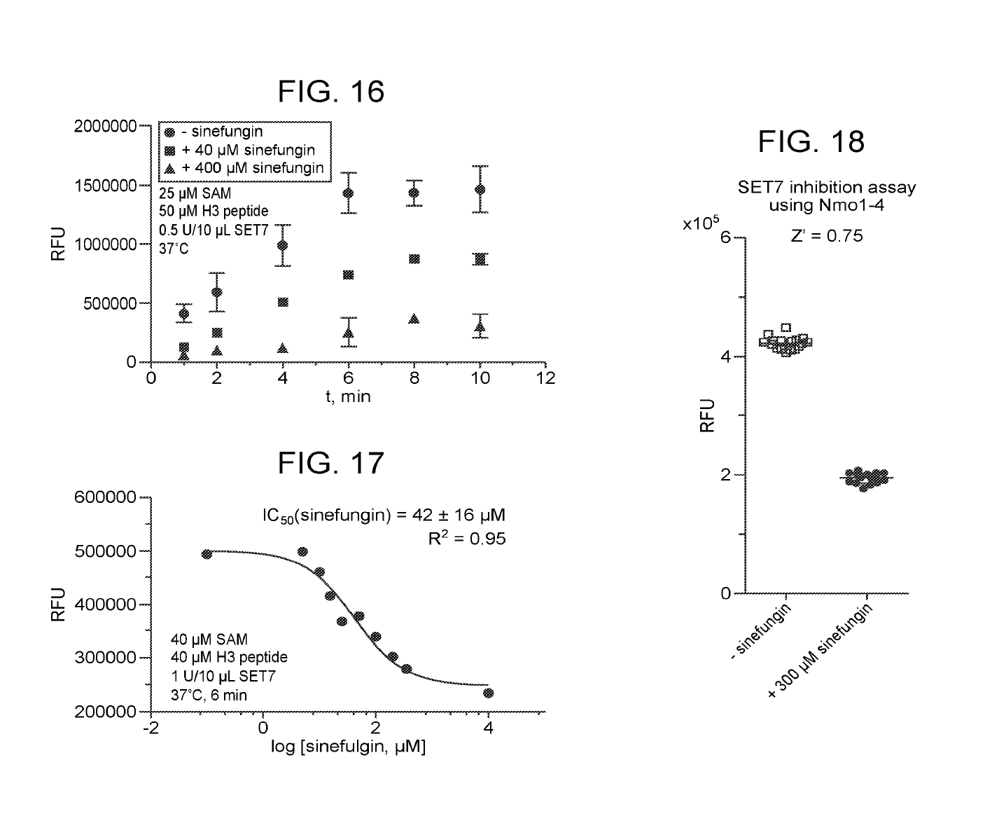

Activity assays using SAH biosensors also provide for characterization and discovery of inhibitors of clinically relevant MTases. Sinefungin, a structural analogue of SAM, is a broad-spectrum MTase inhibitor that competes with SAM in the cofactor-binding pocket. Sinefungin has a similar binding affinity for SET7/9 as SAM, with an IC₅₀ close to the starting concentration of SAM. Accordingly, dose-dependent loss of fluorescence was observed that corresponds to inhibition of SET7/9 by sinefungin (FIG. 15, panel A and FIG. 16). The IC₅₀ was determined to be 26±5 μM, as expected given an initial SAM concentration of 25 μM (FIG. 15, panel A). When the initial concentration of SAM was increased to 40 μM, IC₅₀ also increased to be 42±16 μM (FIG. 17), which confirmed the correlation between sinefungin and SAM.

All of the MTase activity and inhibition assays described above were performed in a 96-well plate and analyzed in a fluorescence plate reader. These conditions lend themselves to high-throughput screening (HTS), a format that would facilitate substrate and drug discovery for MTases of interest. To assess the statistical reliability and performance of HTS methods, a Z′ score is commonly used, where a Z′ score higher than 0.5 is considered to be excellent.^{[27]} To calculate the Z′ score for our activity and inhibition assays, 16 independent replicates of the methylation reactions were manually pipetted into a 384-well plate. The ability to identify substrates and inhibitors using these assays was assessed. Using the Aeh1-4 biosensor, the Z′ scores was 0.74 for the substrate screen and 0.44 for the inhibitor screen (FIG. 15, panel B). In the latter case, the lower Z′ score is due to the biosensor signal not fully decreasing to background even at saturating inhibitor concentrations. This is attributed to sinefungin binding to the biosensor when used at high micromolar concentrations. Thus, it was found that Nmo1-4 biosensor has a Z′ score of 0.75 for the inhibitor screen, because it has a lower affinity to SAM and presumably sinefungin as well (FIG. 18).

In summary, a circular permutation strategy is demonstrated for incorporating pseudoknot-containing aptamers into RNA-based fluorescent biosensors, and a robust MTase assay has been established using these biosensors. The cpSpinach2 apatmer described provides a different scaffold for RNA tagging and biosensor design. Besides the SAH riboswitch, many other natural riboswitch and in vitro selected aptamers contain pseudoknots and they were thought to be incompatible with former biosensor designs (see You et al., Proc. Natl. Acad. Sci. 2015, 112, E2756-E2765. These results show that cpSpinach2 provides a way to overcome this issue, and also indicates that fully circular biosensors would be functional and more stable.

This work also demonstrates how to develop a high-throughput fluorescence plate-reader assay for enzyme activity or inhibition using designed fusions of an aptamer to cpSpinach2. As applied to the MTase enzyme class, the RNA-based biosensors for SAH exhibit comparable selectivity and fluorescence turn-on in comparison to commercial assays that require an antibody and a fluorescent analog of SAH. This system benefits from ease of synthesis and purification, as the RNA-based biosensors are enzymatically synthesized from DNA templates (˜150 nts) in vitro in one step, and the profluorescent dye DFHBI is readily synthesized in three steps.

### Example 4

### In vivo Application of Fluorescent Biosensors to Detect SAH in Live E. coli Cells

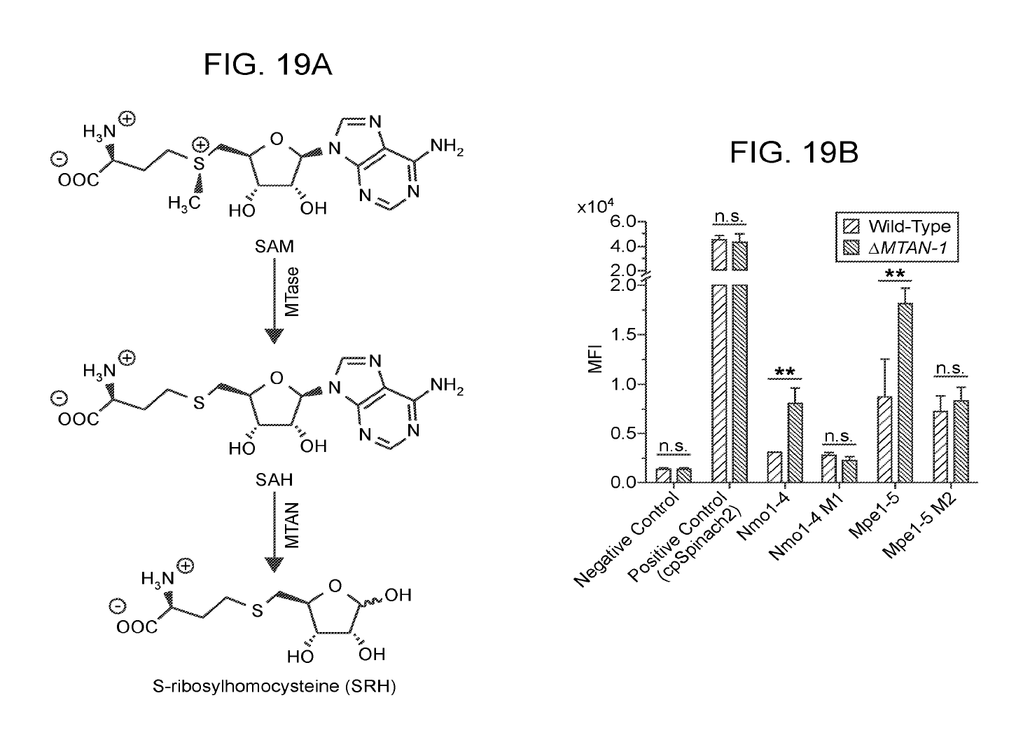

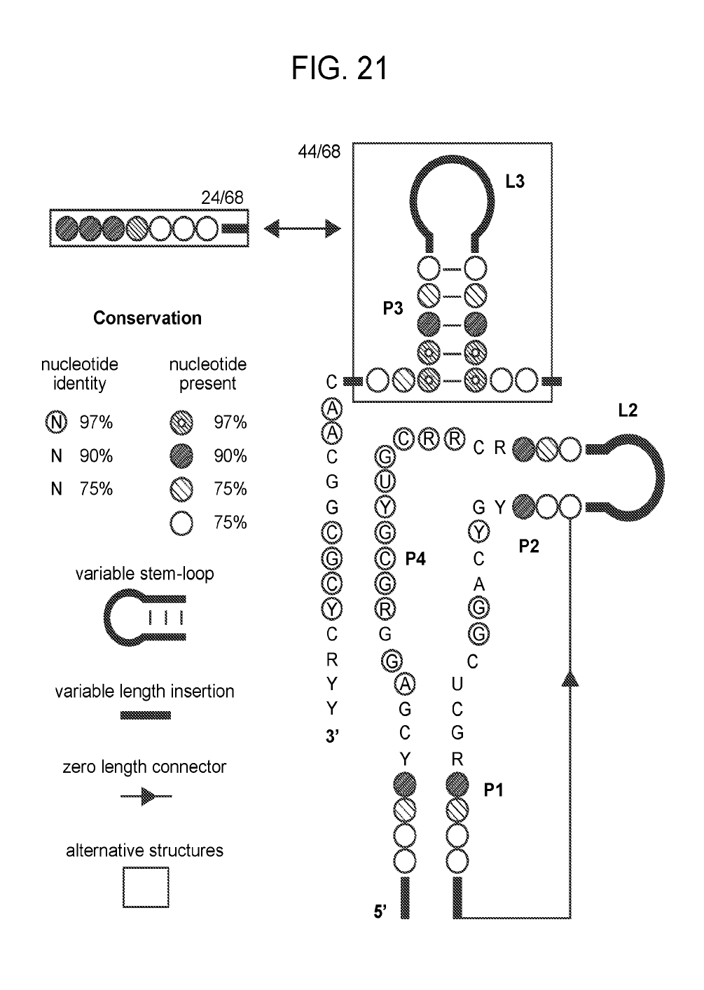

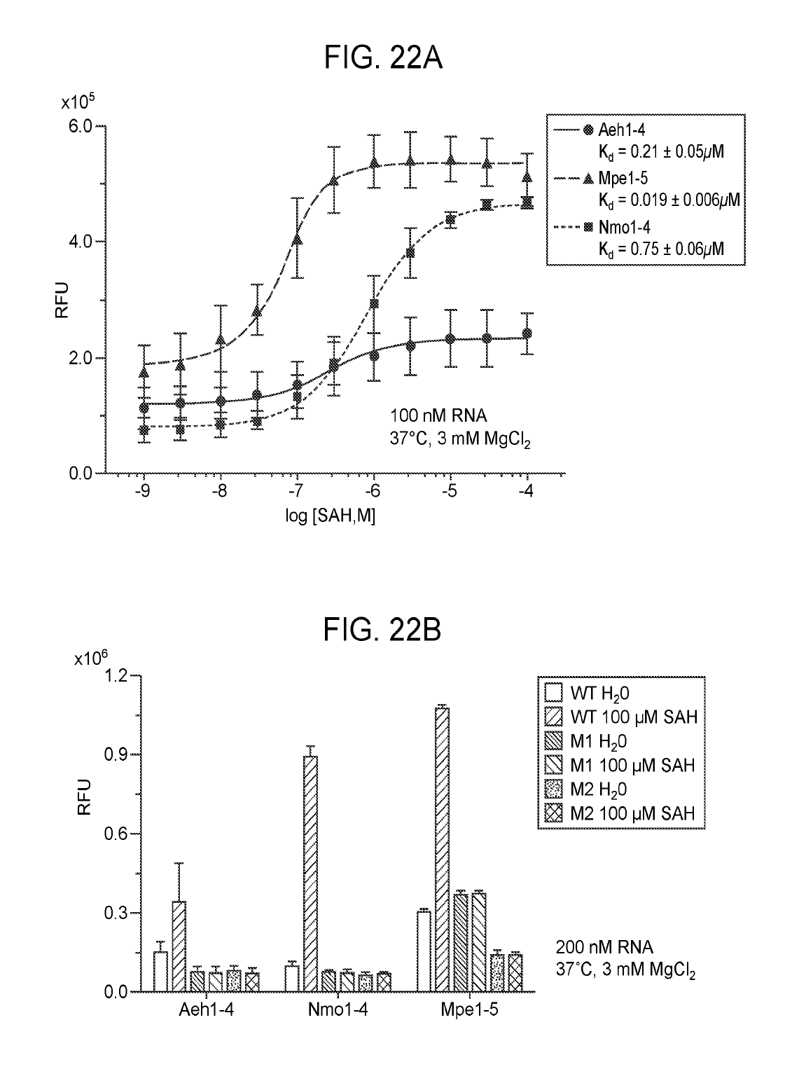

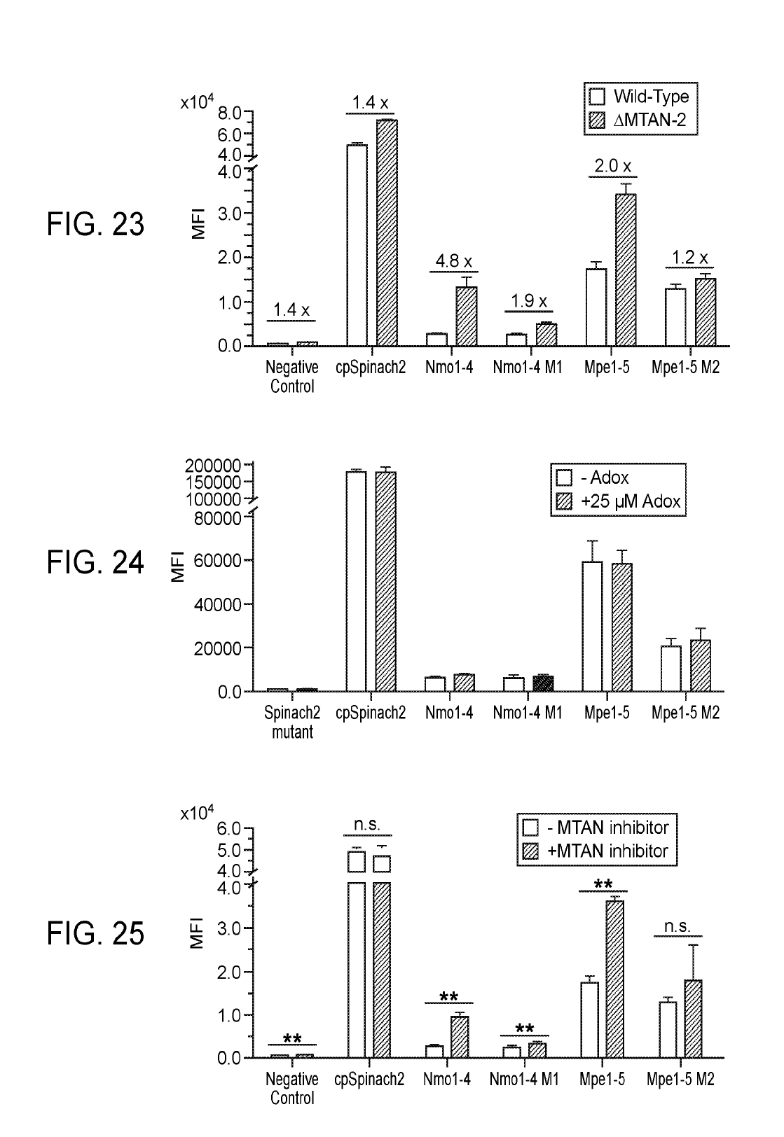

The ability of these RNA-based biosensors to detect SAH in living cells was investigated. In E. coli, SAH is generated from SAM by cellular MTases and then metabolized by MTAN to form S-ribosylhomocysteine (FIG. 19A). Another SAH degradation pathway involves SAH hydrolase (SAHH), but E. coli lacks SAHH activity and does not have any genes homologous to Pseudomonas aeruginosa SAHH. Knock-out of the MTAN gene causes SAH accumulation to levels that are about 50-fold greater than those found in the wild-type E. coli MG1655 strain (1.3 μM for wild-type), while SAM levels remain relatively unaffected (0.4 mM). In order to detect SAH accumulation, both E. coli BL21* wild-type and AMTAN cells expressing SAH biosensors in a tRNA scaffold were analyzed by flow cytometry. Nmo1-4 and Mpe1-5 were chosen for their greater fluorescence brightness and fluorescence turn-on at 37° C. and 3 mM Mg²⁺ , and their corresponding mutants, which disrupt the binding pocket and show no SAH response, were used as controls (FIG. 22, Table 1). As expected, only the functional SAH biosensors exhibited significant increase in fluorescence signal in ΔMTAN versus wild-type cells (FIG. 19B). A second ΔMTAN strain generated from a different target sequence also exhibited a similar trend for SAH detection (FIG. 23). Because the ΔMTAN strain was observed to grow slower than wild-type, cpSpinach2 was also used to evaluate RNA expression; no significant difference in signal was observed for this control, consistent with results for the mutant biosensors. Furthermore, adenosine-2′,3′-dialdehyde (Adox), an inhibitor of SAH hydrolase, does not increase SAH biosensor fluorescence in E. coli cells (FIG. 24), which is consistent with the observation that the enzyme activity was not detected in E. coli.

Given the in vitro measured binding affinities for Nmo1-4 (0.75 μM) and Mpe1-5 (19 nM) (FIG. 22), it was expected that in wild-type E. coli cells, Mpe1-5 should exhibit saturated fluorescence, while Nmo1-4 should show fluorescence turn-on. However, the observations (FIG. 19B and 23) instead showed that SAH levels in wild-type E. coli BL21* cells are close to the middle of the detection range for Mpe1-5 and below the detection limit of Nmo1-4 (FIG. 19B and 23). There are several possible explanations for the observations. First, the biosensors may have poorer binding affinities for SAH under physiological conditions, which are difficult to fully recapitulate in vitro. In fact, it was found that the presence of 3 mM ATP in the in vitro binding reactions slightly reduced the apparent binding affinity of the biosensors for SAH. This effect is likely due to some competitive binding of ATP to the SAH binding pocket, as the riboswitch aptamer has been shown to have weak affinity to ATP. It was found that 3 mM of NAD⁺, which had even weaker affinity to the riboswitch, had no effect on SAH binding affinity. The physiological concentrations of ATP and of NAD⁺ are 3-10 mM and 3 mM, respectively, in E. coli.

Interestingly, the biosensors do not show fluorescence turn-on in direct response to ATP or NAD⁺. Mpe1-5 actually has lower fluorescence in the presence of 10 mM ATP. It was determined that this quenching effect is likely due to competitive binding of ATP to the DFHBI binding pocket, as the Spinach2 aptamer alone showed the same effect. In fact, this result explains the discrepancy between the in vitro binding affinity measured for DFHBI and the inability to saturate fluorescence signal in vivo for Spinach2 and cpSpinach2 (FIG. 5). Importantly, however, the fluorescence signals of Spinach2, cpSpinach2, and the subject biosensors are not fully quenched in vivo, which may reflect that free ATP concentrations are lower than 10 mM.

Another possible explanation is if the cellular concentration of biosensor is much higher than the actual K_{d} value (RNA sensor levels vary from 13.5 μM to 75 μM), the signal becomes proportional to the amount of ligand up to saturation of the biosensor, rather than dependent on K_{d}. The results also show that the relative affinities of different biosensors measured in vitro are consistent with their in vivo performance. Finally, the in vivo data further confirm that the selectivity of our biosensors for SAH over SAM is ˜1000-fold or greater, because little to no background fluorescence activation is observed even though cellular SAM concentrations are high.

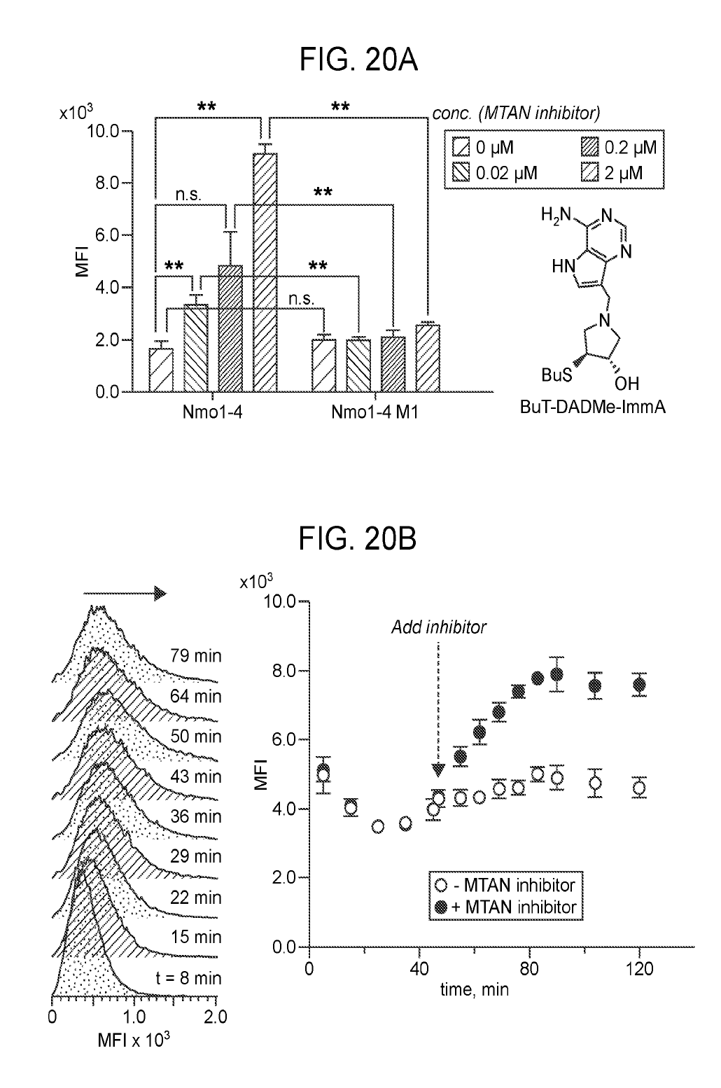

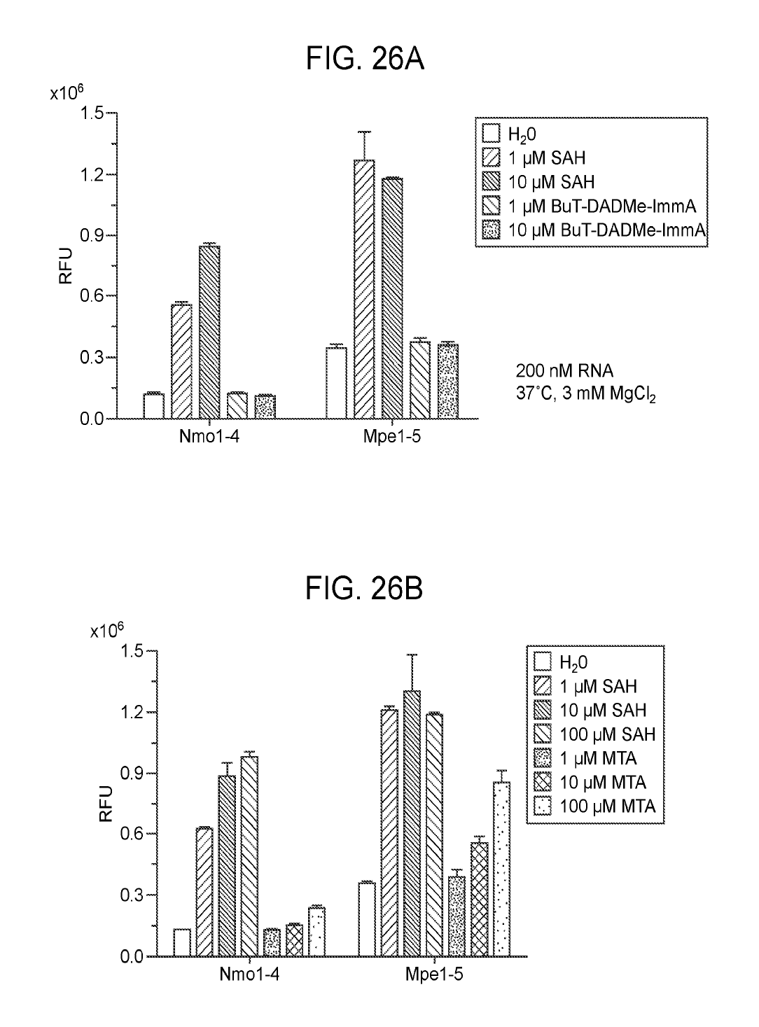

It was next attempted to monitor the dynamic change of SAH levels in live cells. For this purpose, BuT-DADMe-ImmA, a potent MTAN inhibitor, was used to chemically regulate MTAN activity and thus SAH levels. This inhibitor has a dose-dependence inhibition of MTAN in live E. coli cells with an IC₅₀ of 125 nM, and 0.5 μM inhibitor reduced the MTAN activity to a level comparable to an MTAN knockout strain. E. coli BL21* wild-type cells were grown overnight in the presence of various concentrations of MTAN inhibitor. These concentrations are not lethal to the bacteria. Dose-dependent MTAN inhibition led to elevation of SAH levels as detected by Nmo1-4, while the mutant biosensor (Nmo1-4 M1) exhibited no fluorescence increase (FIG. 20A). Importantly, the subject biosensors are highly selective for SAH and thus exhibit no fluorescence response to the MTAN inhibitor and little response to methylthioadenosine (MTA), the other substrate of MTAN, even at high micromolar concentrations (FIGS. 25 and 26). Thus, the dose-dependent fluorescence activation can be attributed to SAH accumulation in treated cells. Gradual SAH accumulation is observed in live cells upon inhibitor treatment, but flow cytometry analysis also reveals that the inhibition effect is not uniform, as the distribution of cellular fluorescence broadens with time (FIG. 20B). Analysis of cpSpinach2 fluorescence under related conditions confirm that biosensor levels remain relatively constant during the time-course. Taken together, these results demonstrate the application of the subject biosensors in detecting and monitoring SAH levels in live E. coli cells.

FIG. 22 shows results of in vitro characterization of SAH biosensors and corresponding mutants under mock physiological conditions. (A) In vitro analysis of biosensor binding affinities for SAH under condition of low concentration of magnesium ion (3 mM). (B) Profile of SAH RNA-based biosensors and their corresponding mutants in response to SAH. Error bars represent standard deviation of 2 independent replicates.

Effect of magnesium concentration on biosensor binding affinity for SAH: Under condition of low concentration of magnesium (3 mM), SAH biosensors Mpe1-5 and Nmo1-4 show high fluorescence response to SAH, with affinity comparable to that measured under high magnesium concentration (10 mM), while Aeh1-4 showed decreased fluorescence turn-on.

Mutants of SAH RNA-based biosensors: No fluorescence response is observed for RNA-based SAH biosensors mutants when high micromolar concentrations of SAH are present. However, background fluorescence of these mutants differs from that of wild-type biosensors (with H₂O). This might be explained by the fact that the mutated nucleotides lie close to the transducer stem of the biosensor (Table 2). Thus, mutation in these positions might affect the stability of the transducer stem, which leads to altered background fluorescence of the biosensors.

FIG. 23 illustrates the live cell detection of SAH accumulation in E. coli BL21*MTAN knockout strain 2 using SAH RNA-based biosensors. The number above each pair of bars represents fold change in fluorescence between wild-type and the MTAN knockout strain. Error bars represent standard deviation of 3 independent biological replicates.

Live cell detection of SAH accumulation in E. coli MTAN-knockout strain 2: Similar to FIG. 4B, Nmo1-4 and Mpe1-5 biosensors detect accumulation of SAH in another E. coli BL21* MTAN knockout strain, though modest fluorescence increases are observed for both negative control (GFP mutant) and samples with cpSpinach2 transcribed (1.4×fluorescence increase). Similar to FIG. 4B, except that a different target sequence for Targetron knockout of the same MTAN gene was used to generate MTAN knockout strain 2. The difference in the exact target sequence might lead to a different extent of off-target effects, which affects the transcription and regulation of RNA levels in the bacterial cells and thus eventually leads to the observed fluorescence increase.

However, it should be noted that despite such background fluorescence increase, both wild-type biosensors exhibited fluorescence changes (4.8× and 2.0×) that were much greater than the background changes (1.4×), as well as greater than their corresponding mutant (1.9× and 1.2×). This observation clearly indicated that both Nmo1-4 and Mpe1-5 are capable of detecting accumulation of SAH in live cells, which is the result of knocking out MTAN in E. coli cells.

Fluorescence of cpSpinach2-expressing E. coli cells grown 16 hrs in auto-induction media, then pelleted and re-suspended in M9 minimal media without RNA transcription inducers.

RNA stability in E. coli: For the time-course experiment shown in FIG. 4D, E. coli cells from overnight culture (16 h post-inoculation and induction) were pelleted and re-suspended in M9 minimal media without transcriptional inducers (lactose). To analyze the stability of RNA levels under these conditions, E. coli cells expressing cpSpinach2 were processed using the same procedures and fluorescence was monitored for 4 h. Fluorescence readings remained stable for up to 3 h in M9 minimal media. Therefore, we expect that the related biosensor levels would be stable in the same time-frame, so the fluorescence change shown in FIG. 4D reflects the dynamic change in SAH levels.

### Materials and Methods

Bioinformatic Analysis of SAH Riboswitches

52 SAH riboswitches seed sequences from Rfam database (accession RF01051, rfam.xfam.org) (Nawrocki et al., Nucleic Acids Research 2015, 43, D130-137) were analysed. Discarding all metagenomic sequences among them gave 41 sequences. Jalview software was used to align these sequences and to identify each riboswitch's P2 stem. Sequences with P2 stems shorter than 4 base pairs were discarded due to their likely instability when fused to cpSpinach2. Among the remaining 39 sequences, a pairwise alignment was carried out using Jalview. If more than 85% of the two sequences were identical, one sequence was discarded. After this analysis, 29 sequences were chosen for phylogenetic screening.

Reagents and Oligonucleotides

DNA oligonucleotides for biosensor constructs were purchased. S-Adenosyl-L-methionine (SAM), S-adenosyl-L-homocysteine (SAH), DL-homocysteine, adenosine and L-methionine were purchased from Sigma-Aldrich (St Louis, Mo.). Sinefungin was purchased from Santa Cruz Biotech (Santa Cruz, Calif.). DFHBI and DFHBI-1T were either purchased from Lucerna (New York, N.Y.) or were synthesized (e.g., by the procedure of Song et al., Journal of the American Chemical Society 2014, 136, 1198-1201) and were stored as a 10 mM stock in DMSO. Commercially available reagents were used without further purification. H3 peptide was either purchased or synthesized. T7 RNA polymerase, Phusion DNA polymerase, DNA methyltransferase M.Sssl and protein methyltransferase SET7/9 were purchased from New England Biolabs Inc (Ipswich, Mass.). Chemically competent BL21 (DE3) Star cells were purchased from Life Technologies (Carlsbad, Calif.).

In vitro Transcription

DNA templates for in vitro transcription were prepared through PCR amplification using Phusion DNA polymerase (NEB) from sequence-confirmed plasmids or Ultramer oligonucleotides (for screening experiment only) using primers that added the T7 polymerase promoter sequence. PCR products were purified either by a 96-well format ZR-96 DNA Clean-up kit (Zymo Research) for screening experiments or by QlAquick PCR purification kit (Qiagen) for characterization and application experiments. Templates were then transcribed using T7 RNA polymerase in 40 mM Tris-HCl, pH 8.0, 6 mM MgCl₂, 2 mM spermidine, and 10 mM DTT. RNAs were either purified by a 96-well format ZR-96 Clean & Concentrator (Zymo Research) or by denaturing (7.5 M urea) 6% PAGE. RNAs purified from PAGE were subsequently extracted from gel pieces using Crush Soak buffer (10 mM Tris-HCl, pH 7.5, 200 mM NaCl and 1 mM EDTA, pH 8.0). RNAs were precipitated with ethanol, dried, and then resuspended in TE buffer (10 mM Tris-HCl pH 8.0, 1 mM EDTA). Accurate measurement of RNA concentration was determined by measuring the absorbance at 260 nm after performing a hydrolysis assay to eliminate the hypochromic effect due to secondary structure in these RNAs (see Wilson et al., RNA 2014, 20, 1153-1160).

Determination of Fluorescence Quantum Yields and Apparent Binding Affinities

In vitro analysis of RNA-DFHBI complex quantum yields and binding affinity for DFHBI and quantum yields was carried out as described by Babendure et al., Journal of the American Chemical Society 2003, 125, 14716-14717. Briefly, all quantum yields were determined by comparing the integral of the background-subtracted emission spectra of DFHBI or RNA-DFHBI complex in buffered water (40 mM HEPES, pH 7.5, 125 mM KCl, 10 mM MgCl₂) with the corresponding integral obtained from the background-subtracted emission spectra of Acridine Yellow in ethanol. The absorbance was measured at 448 nm and the fluorescence emission spectra was measured with 448 nm excitation for three different concentrations of the Acridine Yellow standard. The absolute quantum yield of Acridine Yellow was taken to be 0.47 as reported by Olmsted, Journal of Physical Chemistry 1979, 83, 2581-2584. The refractive index (η) was taken as 1.36 for ethanol and 1.333 for water (E. Hecht, Optics (4th edition), 2001). UV absorbance and fluorescence spectra were recorded on a Shimadzu 2501 Spectrophotometer (Shimadzu) or a Quantamaster Master 4 L-format scanning spectrofluorometer (Photon Technologies International) equipped with an LPS-220B 75-W xenon lamp and power supply, A-1010B lamp housing with integrated igniter, switchable 814 photon counting/analog photomultiplier detection unit, and MD5020 motor driver.

Quantum yields for DFHBI or RNA-DFHBI complex were determined by measuring the apparent quantum yields (Φ_{sample.app}) as a function of increasing total RNA (C_{total RNA}) at a fixed concentration of total DFHBI (C_{DFHBI}). The measurements were performed in binding buffer (40 mM HEPES, pH 7.5, 125 mM KCl, and 10 mM MgCl₂) at 30° C. The RNA was renatured in buffer at 70° C. for 3 min and cooled to ambient temperature for 5 min prior to addition to the reaction solution. The DFHBI concentration was fixed at 10 or 20 μM in different replicates. The apparent quantum yield is described using the following equation:

$$\begin{matrix}{Φ}_{sample , app} = {Φ}_{ST} ( \frac{{FI}_{sample} \times {A}_{ST}}{{FI}_{ST} \times {A}_{sample}} ) ( \frac{{n}_{water}^{2}}{{n}_{ethanol}^{3}} ) \\ = {Φ}_{ST} ( \frac{{FI}_{sample}}{{Grad}_{ST} \times {A}_{sample}} ) ( \frac{{n}_{water}^{2}}{{n}_{ethanol}^{3}} )\end{matrix}$$

Then, the apparent quantum yield is related to the dissociation constant K_{d} and concentration of DFHBI by the following equation:

$$\begin{matrix}{Φ}_{sample , app} = \frac{{Φ}_{D} \times [ DFHBI ] + {Φ}_{RD} \times [ RD ]}{{c}_{DFHBI}} \\ = {Φ}_{D} + \frac{( {Φ}_{RD} - {Φ}_{D} ) \times [ RD ]}{{c}_{DFHBI}} \\ = [ \frac{\begin{matrix}{c}_{total RNA} + {K}_{d} + {c}_{DFHBI} - \\ \sqrt{\begin{matrix}{( {c}_{total RNA} + {K}_{d} + {c}_{DFHBI} )}^{2} - \\ 4 \times {c}_{total RNA} \times {c}_{DFHBI}\end{matrix}}\end{matrix}}{2 {c}_{total RNA}} ] ( {Φ}_{sample} - {Φ}_{D} ) + {Φ}_{D}\end{matrix}$$

Thus, the dissociation constants K_{d} and the quantum yield (Φₛₐₘₚₗₑ) of RNA-DFHBI complexes were determined by least squares fitting to the above equation in Graphpad Prism software.

Determination of Melting Temperatures of RNA-Dye Complexes

In vitro analysis of RNA-DFHBI-1T complex melting temperatures was carried out using methods described by R. L. Strack, M. D. Disney, S. R. Jaffrey, Nature Methods 2013, 10, 1219-1224. Briefly, in a buffer containing 40 mM HEPES, pH 7.5, 125 mM KCl, indicated concentrations of MgCl₂ were added 200 nM RNA and 10 μM DFHBI-1T. The RNA was renatured in buffer at 70° C. for 3 min and cooled to ambient temperature for 5 min prior to addition to the reaction solution. The temperature-dependent fluorescence was measured on a Biorad CFX96™ real-time PCR detection system, spanning from 15° C. to 65° C. with a step increment of 0.5° C./step. Melting temperatures were obtained as the corresponding temperature of half-maximal fluorescence.

General Procedures for in vitro Fluorescence Assays

In vitro fluorescence assays were carried out in a buffer containing 40 mM HEPES, pH 7.5, 125 mM KCl. Other conditions, including temperature, concentrations of MgCl₂, DFHBI, ligand or RNA, were varied in different experiments and are indicated in the figures. The RNA was renatured in buffer at 70° C. for 3 min and cooled to ambient temperature for 5 min prior to addition to the reaction solution. DFHBI was added to a solution containing buffer and ligand, and then RNA was added at the end before fluorescence measurement. Binding reactions were performed in 100 μL volumes and were incubated at the indicated temperature in a Corning Costar 3915 96-well black plate until equilibrium was reached, which in some cases takes 30 to 60 minutes. The fluorescence emission was measured using a Molecular Devices SpectraMax Paradigm Multi-Mode detection platform plate reader (Sunnyvale, Calif.) with the following instrument parameters: 448 nm excitation, 506 nm emission.

The folding assay was carried out using the methods described by Strack et al., Nature Methods 2013, 10, 1219-1224, with the following conditions: 30° C. and 10 mM MgCl₂. In the “limiting RNA” measurements, RNA concentration was 80 nM while DFHBI was 8 μM. In the “limiting DFHBI” measurements, DFHBI concentration was 80 nM while RNA was 8 μM.

In order to screen the SAH riboswitch phylogenetic library, fluorescence assays were performed with the following conditions: 37° C., 10 mM MgCl₂, and varied ligand concentrations (0, 1, 100 μM for SAH or 100 μM for SAM), 200 nM RNA and 10 μM DFHBI.

Binding Affinity Analysis of RNA-DFHBI Complexes

For experiments to measure K_{d} of Spinach2 or cpSpinach2 aptamers, fluorescence assays were run as described above, with the following conditions: 30° C. or 37° C., 10 mM MgCl₂ and 30 nM RNA. The DFHBI concentration (C_{DFHBI}) was varied as the concentration of RNA (C_{total RNA}) was held constant, and the fluorescence of the sample with DFHBI but no RNA was subtracted as background to determine relative fluorescence units. The concentration [RNA-DFHBI] of the RNA-DFHBI complex is given by:

$$\begin{matrix}[ RNA \cdot DFHBI ] = \frac{[ RNA ] [ DFHBI ]}{{K}_{d}} \\ = \frac{\begin{matrix}( {c}_{total RNA} - [ RNA \cdot DFHBI ] ) \\ ( {c}_{DFHBI} - [ RNA \cdot DFHBI ] )\end{matrix}}{{K}_{d}}\end{matrix}$$

where [RNA] and [DFHBI] are the concentrations of free RNA and free dye, respectively, and K_{d} is the dissociation constant for the complex. Solving the quadratic equation for [RNA-DFHBI] gives:

$$[ RNA \cdot DFHBI ] = \frac{\begin{matrix}{c}_{total RNA} + {K}_{d} + {c}_{DFHBI} - \\ \sqrt{\begin{matrix}{( {c}_{total RNA} + {K}_{d} + {c}_{DFHBI} )}^{3} - \\ 4 \times {c}_{total RNA} \times {c}_{DFHBI}\end{matrix}}\end{matrix}}{2}$$

Assuming Fₘᵢₙ is the background fluorescence when no RNA is bound by DFHBI (measured as DFHBI only sample), and Fₘₐₓ is the fluorescence when all the RNA is bound by DFHBI (measured as the maximum fluorescence at saturating RNA concentrations), the ratio between the concentrations of RNA-DFHBI complex and total RNA is:

$$\frac{[ RNA \cdot DFHBI ]}{{c}_{total RNA}} = \frac{F - {F}_{m i n}}{{F}_{m ax} - {F}_{m i n}}$$

Thus the measured fluorescence for varied concentration of RNA-DFHBI complex is given as:

$$\begin{matrix}F = \frac{[ RNA \cdot DFHBI ]}{{c}_{total RNA}} \times ( {F}_{m ax} - {F}_{m i n} ) + {F}_{m i n} \\ = [ \frac{\begin{matrix}{c}_{total RNA} + {K}_{d} + {c}_{DFHBI} - \\ \sqrt{\begin{matrix}{( {c}_{total RNA} + {K}_{d} + {c}_{DFHBI} )}^{2} - \\ 4 \times {c}_{total RNA} \times {c}_{DFHBI}\end{matrix}}\end{matrix}}{2 \times {c}_{total RNA}} ] \times \\ ( {F}_{ma x} - {F}_{m i n} ) + {F}_{m i n}\end{matrix}$$

The dissociation constants K_{d} were determined by least squares fitting to the above equation in Graphpad Prism software.

Binding Affinity Analysis of SAH Biosensors

To measure the binding affinities of SAH biosensors, fluorescence assays were performed with the following conditions: 37° C., 10 mM MgCl₂, 30 nM RNA, and 10 μM DFHBI. The SAH concentration (c_{SAH}) was varied, and the fluorescence of the sample with DFHBI but no RNA was subtracted as background to determine relative fluorescence units. The ratio between the fraction of biosensor that is bound by SAH and the total SAH biosensor concentration is given as:

$$ratio bound = \frac{[ RNA \cdot SAH \cdot DFHBI ]}{{c}_{total RNA}} = \frac{F - {F}_{m i n}}{{F}_{m ax} - {F}_{m i n}}$$

The dissociation constants K_{d} were determined by least squares fitting to the following equation in Graphpad Prism software:

$$\begin{matrix}ratio bound = \frac{[ RNA \cdot SAH \cdot DFHBI ]}{{c}_{total RNA}} \\ = [ \frac{\begin{matrix}{c}_{total RNA} + {K}_{d} + {c}_{SAH} - \\ \sqrt{\begin{matrix}{( {c}_{total RNA} + {K}_{d} + {c}_{SAH} )}^{2} - \\ 4 \times {c}_{total RNA} \times {c}_{SAH}\end{matrix}}\end{matrix}}{2 \times {c}_{total RNA}} ]\end{matrix}$$

HPLC Purification of SAM Stocks

0.5 mM SAM (Sigma-Aldrich) in 0.05% TFA/ddH₂O solution was injected and loaded into Agilent Polaris 5 C-18-A 250×10.0 mm column in an Agilent 1260 Infinity LC system for purification. Method of the separation was as follows: 0-12 min, 0-10% solution B; 12-15 min, 10-50% solution B. (Solution A: H₂O+0.05% TFA; solution B: Acetonitrile+0.05% TFA). Collected fractions were lyophilized to give products as white solid.

LC-MS Analysis of SAM Stocks

10 μL of 0.5 mM SAM (Sigma-Aldrich) in ddH₂O solution was injected and loaded into Poroshell 120 C-18 column in an Agilent 1260 Infinity LC-MS system for analysis. Method of the separation was as follows: 0-10 min, 5-95% solution B. (Solution A: H₂O+0.05% TFA; solution B: Acetonitrile+0.05% TFA).

In vivo Fluorescence Assays by Flow Cytometry

Preparation of cell samples for flow cytometry was carried out by inoculating 3 mL of LB/carb media with 150 μL of an overnight culture of cells containing either the pET31b-Spinach2 or pET31b-cpSpinach2 plasmid. Cells were grown aerobically to an 0D₆₀₀˜0.5-0.6, then induced with 1 mM IPTG at 37° C. for 2 hrs. Cell density was measured by OD₆₀₀, and assuming that there are 1×10⁹ cells/mL for 1 AU, 4×10⁸ cells were sampled and pelleted at room temperature for 4 min at 3,700 rcf, washed once with PBS media at pH 7.0, and then resuspended in PBS media containing 100 μM DFHBI. Cellular fluorescence was measured for 30,000 cells using either a Life Technologies Attune N×T Acoustic Focusing Cytometer or a BD Fortessa X20 flow cytometer with BD FACS Sortware (Version 1.0.0.650). The BD Fortessa X20 flow cytometer is located in the Flow Cytometry Core Facilities at UC Berkeley.

DNA Methyltransferases (MTase) M.SssI Activity Assay

To initiate the enzyme reaction, 5 μL of dsDNA substrate (5′-GAGCCCGTAAGCCCG-TTCAGGTCG-3′) (SEQ ID NO:131) was added to 5 μL of 2×enzyme reaction solution containing M.Sssl, SAM, and NEBuffer 2. Final concentrations are 40 μM dsDNA, 0.4 U/μL M.Sssl, 20 μM SAM, and 1×NEBuffer 2 (20 mM Tris, pH 7.9, 20 mM MgCl₂, and 100 mM NaCl). The reaction solution was incubated at 37° C. for 3 hours to allow the enzyme reaction to proceed, then incubated at 65° C. for 20 min for heat inactivation.

To initiate the fluorescent biosensor reaction, 10 μL of the enzyme reaction was added to 90 μL of biosensor reaction solution containing renatured RNA, DFHBI, and buffer. Final concentrations are 200 nM RNA, 1 μM DFHBI, 40 mM HEPES, pH 7.5, 125 mM KCl, 10 mM MgCl₂, and 0.1x NEBuffer2 (2 mM Tris, pH 7.9, 2 mM MgCl₂, and 10 mM NaCl). For the SAH control, SAH was added to a final concentration of 1 μM in place of the enzyme reaction. In vitro fluorescence assays were conducted as described above.

Protein MTase SET7/9 Activity, Kinetic and Inhibition Assay

To initiate the enzyme reaction, 5 μL of H3 peptide substrate (N′-ARTKQTARKSTGGKAPRKQ-LAGGKKC-C′) (SEQ ID NO:132) was added to 5 μL of 2×enzyme reaction solution containing SET7/9, SAM, and NEB HMT buffer. Generally, final concentrations are 50 μM H3 peptide, 0.1 U/μL SET7/9, 25 μM SAM, and lx NEB HMT buffer (50 mM Tris, pH 9.0, 5 mM MgCl₂, and 4 mM DTT). The reaction solution was incubated at 37° C. for 2 hours to allow the enzyme reaction to proceed.

For the kinetics assay, the reaction conditions are the same as described above except 0.05 U/μL SET7/9 was used, and the enzyme reaction was heat inactivated at different time points. For the inhibition assay, the reaction conditions are the same as described in kinetics assay except varied concentrations of sinefungin was added to the enzyme reaction solution, the reaction solution was incubated at 37° C. for 6 min (see FIG. 16), then heat inactivated by incubation at 65° C. for 15 min. For the inhibition assay shown in FIG. 17, the reaction conditions are the same as described above except 40 μM H3 peptide, 0.1 U/μL SET7/9, 40 μM SAM were used instead.

To initiate the fluorescent biosensor reaction, 10 μL of the enzyme reaction was added to 90 μL of biosensor reaction solution containing renatured RNA, DFHBI, and buffer. Final concentrations are 200 nM RNA, 10 μM DFHBI, 40 mM HEPES, pH 7.5, 125 mM KCl, 10 mM MgCl₂, and 0.1×NEB HMT buffer (5 mM Tris, pH 9.0, 0.5 mM MgCl₂, and 0.4 mM DTT). For the SAH control, SAH was added to a final concentration of 0.5 μM in place of the enzyme reaction. In vitro fluorescence assays were conducted as described above.

High-Throughput Screening (HTS) Assay of SET7/9 Activity and Inhibition

For HTS experiments, samples were dispensed into a Grenier 781077 384-well flat bottom black plate using an Eppendorf Repeater Xstream pipetter to dispense reaction components. Z′ scores were calculated from 16 well replicates in the 384-well plate.

For the HTS SET7/9 activity assay, 3 μL of H3 peptide or water was added to 9 μL of reaction solution containing SET7/9 MTase, SAM, and NEB HMT Reaction Buffer in each well. Final concentrations are: 0.25 U/10 μL SET7/9, 25 μM SAM, 50 μM H3 peptide and 1×NEB HMT Reaction Buffer (50 mM Tris, pH 9.0, 5 mM MgCl₂, and 4 mM DTT). After 30 min incubation at 37° C., to initiate the fluorescent biosensor reaction, 18 μL of biosensor reaction solution containing renatured RNA, DFHBI, and buffer was added to each well. Final concentrations are 200 nM RNA, 10 μM DFHBI, 40 mM HEPES, pH 7.5, 125 mM KCl, 10 mM MgCl₂, and 0.4×NEB HMT buffer (20 mM Tris, pH 9.0, 2 mM MgCl₂, and 1.6 mM DTT). In vitro fluorescence assays were conducted as described above.

For the HTS SET7/9 inhibition assay, 4.5 μL of sinefungin or water was added to 8 μL of reaction solution containing SET7/9 MTase, and NEB HMT Reaction Buffer in each well. After 15 minutes incubation at 37° C., methylation reaction was initiated by adding 2.5 μL solution containing both SAM and H3 peptide. Final concentrations are: 0.25 U/10 μL SET7/9, 25 μM SAM, 50 μM H3 peptide, 300 μM sinefungin and 1×NEB HMT Reaction Buffer (50 mM Tris, pH 9.0, 5 mM MgCl₂, and 4 mM DTT). After 30 min incubation at 37° C., to initiate the fluorescent biosensor reaction, 15 μL of biosensor reaction solution containing renatured RNA, DFHBI, and buffer was added to each well. Final concentrations are 200 nM RNA, 10 μM DFHBI, 40 mM HEPES, pH 7.5, 125 mM KCl, 10 mM MgCl₂, and 0.5×NEB HMT buffer (25 mM Tris, pH 9.0, 2.5 mM MgCl₂, and 2 mM DTT). In vitro fluorescence assays were conducted as described above.

Generation of MTAN Knockout Strain of E. coli BL21*

A Targetron® Gene Knockout System (Sigma-Aldrich) was used to knock out the MTAN gene from E. coli BL21*, following manufacturer protocols.

Live Cell Fluorescence Assays Using SAH Biosensors

Preparation of cell samples for flow cytometry was carried out by inoculating 2 mL of auto-induction media/Carb (F. Studier, Protein Expression and Purification 2005, 41, 207-234) with E. coli BL21* cells from colonies grown on an LB/Carb culture plate, containing pET31b plasmids encoding RNA aptamers. For experiments involving MTAN inhibitor (BuT-DADMe-ImmA) or SAHH inhibitor (adenosine-2′, 3′-dialdehyde) treatment, the inhibitor was added directly to the media to reach the indicated concentration before cell inoculation. Cells were then grown aerobically overnight to saturation (0D₆₀₀ varied from 5 to 10) and diluted to OD₆₀₀˜0.01-0.05 in M9 minimal media (pH ˜6.0) with 100 μM DFHBI-1T. Cellular fluorescence was measured for 30,000 cells using a Life Technologies Attune N×T Acoustic Focusing Cytometer.

*Instead of normal LB media with IPTG induction, auto-induction media was used due to its effectiveness in avoiding toxicity caused by high concentrations of IPTG and in order to reduce the variation between independent biological replicates. Such reduction in the variation might be a result of the auto-induction mechanism, since the induction is initiated at similar cell density among varied replicate samples, and cells are able to grow to saturation with consistently high density.

Real-Time Monitoring of MTAN Inhibition in E. coli Using SAH Biosensors

For real-time monitoring of MTAN inhibition by BuT-DADMe-ImmA, wild type E. coli BL21* containing pET31b plasmid encoding SAH biosensor was inoculated and grown overnight in auto-induction medialCarb as described above. Then, on reaching saturation (OD₆₀₀ varied from 5 to 10), bacterial cells were pelleted at room temperature for 3 min at 1000×g, washed once with M9 minimal media at pH 6.0, and then resuspended in M9 minimal media containing 100 μM DFHBI-1T. Then cells were incubated at 37° C. and aliquots ware taken out for flow cytometry measurements as described above at indicated time points. When fluorescence stayed stable over at least 20 min, MTAN inhibitor was added to a final concentration of 1 μM, and then the cells were incubated at 37° C. and aliquots ware analyzed as described above.

TABLE 2
Sequences used in the examples described herein. Bold and underlined
sequences indicated cpSpinach2 sequence used in SAH biosensors.
Italic and underlined sequences indicated mutation positions.

| Name | Sequence (5′ to 3′) |
|---|---|
| Spinach2 | GATGTAACTG AATGAAATGG TGAAGGACGG GTCCAGTAGG |
| | CTGCTTCGGC AGCCTACTTG TTGAGTAGAG TGTGAGCTCC |
| | GTAACTAGTT ACATC (SEQ ID NO: 92) |
| cpSpinach2 | GCAGCCTACT TGTTGAGTAG AGTGTGAGCT CCGTAACTAG |
| | TTACATCGCA AGATGTAACT GAATGAAATG GTGAAGGACG |
| | GGTCCAGTAG GCTGC (SEQ ID NO: 93) |
| Aeh1-4 | CGCCCCGAGG AGCGCTGCAA CAGGTTGTTG AGTAGAGTGT |
| | GAGCTCCGTA ACTAGTTACA TCGCAAGATG TAACTGAATG |
| | AAATGGTGAA GGACGGGTCC ACCTGCCAGG CTCGGGGATG |
| | GCGCCCCATC GTTCCAACGG CGCTCAGCGA AA (SEQ ID NO: 44) |
| Aeh1-4 M1 | CGCCCCGAGG AGCGCTGCAA CAGGTTGTTG AGTAGAGTGT |
| | GAGCTCCGTA ACTAGTTACA TCGCAAGATG TAACTGAATG |
| | AAATGGTGAA GGACGGGTCC ACCTGCCTCG CTCGGGGATG |
| | GCGCCCCATC GTTCCAACGG CGCTCAGCGA AA (SEQ ID NO: 94) |
| Aeh1-4 M2 | CGCCCCGAGG AGCGCTCCAA CAGGTTGTTG AGTAGAGTGT |
| | GAGCTCCGTA ACTAGTTACA TCGCAAGATG TAACTGAATG |
| | AAATGGTGAA GGACGGGTCC ACCTGCCAGG CTCGGGGATG |
| | GCGCCCCATC GTTCCAACGG CGCTCAGCGA AA (SEQ ID NO: 95) |
| Nmo1-4 | CTCGCCGAGG AGCGCTGCAA CAGGTTGTTG AGTAGAGTGT |
| | GAGCTCCGTA ACTAGTTACA TCGCAAGATG TAACTGAATG |
| | AAATGGTGAA GGACGGGTCC ACCTGCCAGG CTCGGCGATT |
| | ATCGGGACCT TTAAACCAAC GGCGCTCGGT TCAG (SEQ ID NO: 64) |
| Nmo1-4 M1 | CTCGCCGAGG AGCGCTGCAA CAGGTTGTTG AGTAGAGTGT |
| | GAGCTCCGTA ACTAGTTACA TCGCAAGATG TAACTGAATG |
| | AAATGGTGAA GGACGGGTCC ACCTGCCTCG CTCGGCGATT |
| | ATCGGGACCT TTAAACCAAC GGCGCTCGGT TCAG (SEQ ID NO: 96) |
| Nmo1-4 M2 | CTCGCCGAGG AGCGCTCCAA CAGGTTGTTG AGTAGAGTGT |
| | GAGCTCCGTA ACTAGTTACA TCGCAAGATG TAACTGAATG |
| | AAATGGTGAA GGACGGGTCC ACCTGCCAGG CTCGGCGATT |
| | ATCGGGACCT TTAAACCAAC GGCGCTCGGT TCAG (SEQ ID NO: 97) |
| Mpe1-5 | ACTTTCGAGG AGCGTTGCAA CTCCCTTGTT GAGTAGAGTG |
| | TGAGCTCCGT AACTAGTTAC ATCGCAAGAT GTAACTGAAT |
| | GAAATGGTGA AGGACGGGTC CAGGGAGCCA GGCTCGAAAG |
| | TCATCCCCCG ATGCAACGGC GCTCACCCGC A (SEQ ID NO: 39) |
| Mpe1-5 M1 | ACTTTCGAGG AGCGTTGCAA CTCCCTTGTT GAGTAGAGTG |
| | TGAGCTCCGT AACTAGTTAC ATCGCAAGAT GTAACTGAAT |
| | GAAATGGTGA AGGACGGGTC CAGGGAGCCT CGCTCGAAAG |
| | TCATCCCCCG ATGCAACGGC GCTCACCCGC A (SEQ ID NO: 98) |
| Mpe1-5 M2 | ACTTTCGAGG AGCGTTCCAA CTCCCTTGTT GAGTAGAGTG |
| | TGAGCTCCGT AACTAGTTAC ATCGCAAGAT GTAACTGAAT |
| | GAAATGGTGA AGGACGGGTC CAGGGAGCCA GGCTCGAAAG |
| | TCATCCCCCG ATGCAACGGC GCTCACCCGC A (SEQ ID NO: 99) |
| T7 promoter | CCAAGTAATA CGACTCACTA TAGG (SEQ ID NO: 100) |
| T7 terminator | TAGCATAACC CCTTGGGGCC TCTAAACGGG TCTTGAGGGG TTTTTTG |
| | (SEQ ID NO: 101) |
| 5′ tRNA scaffold | CGGCCGCGGG TCCAGGGTTC AAGTCCCTGT TCGGGCGCCA (SEQ ID |
| | NO: 133) |
| 3′ tRNA scaffold | GCCCGGATAG CTCAGTCGGT AGAGCAGCGG CCG (SEQ ID NO: 113) |

TABLE 3
Sequences used in the phylogenetic screen. Bold sequences indicate cp5pinach2
sequences used in the SAH biosensors. Italic and underlined sequences indicate
the transducer stern of the biosensors.

| Name | Sequence (5′ to 3′) |
|---|---|
| Aeh1-4 | CGCCCCGAGG AGCGCTGCAA CAGGTTGTTG AGTAGAGTGT GAGCTCCGTA |
| | ACTAGTTACA TCGCAAGATG TAACTGAATG AAATGGTGAA GGACGGGTCC |
| | ACCTGCCAGG CTCGGGGATG GCGCCCCATC GTTCCAACGG CGCTCAGCGA AA |
| | (SEQ ID NO: 44) |
| Aeh1-5 | CGCCCCGAGG AGCGCTGCAA CAGGGTTGTT GAGTAGAGTG TGAGCTCCGT |
| | AACTAGTTAC ATCGCAAGAT GTAACTGAAT GAAATGGTGA AGGACGGGTC |
| | CACCCTGCCA GGCTCGGGGA TGGCGCCCCA TCGTTCCAAC GGCGCTCAGC |
| | GAAA (SEQ ID NO: 34) |
| Azo1-4 | CCTGCCGAGG AGCGCTGCGA CCCGTTGTTG AGTAGAGTGT GAGCTCCGTA |
| | ACTAGTTACA TCGCAAGATG TAACTGAATG AAATGGTGAA GGACGGGTCC |
| | ACGGGCCAGG CTCGGCAACC GAACAACGGC GCTCGCAAAC C (SEQ ID NO: 45) |
| Azo1-5 | CCTGCCGAGG AGCGCTGCGA CCCGATTGTT GAGTAGAGTG TGAGCTCCGT |
| | AACTAGTTAC ATCGCAAGAT GTAACTGAAT GAAATGGTGA AGGACGGGTC |
| | CACCGGGCCA GGCTCGGCAA CCGAACAACG GCGCTCGCAA ACC (SEQ ID NO: |
| | 35) |
| Bgl1-4 | TTTCCGAGGA GCGTTGCGAC GGGTTGTTGA GTAGAGTGTG AGCTCCGTAA |
| | CTAGTTACAT CGCAAGATGT AACTGAATGA AATGGTGAAG GACGGGTCCA |
| | CCCGCCAGGC TCGGAAATCT TCACCAGGCG GTGGCCCG (SEQ ID NO: 46) |
| Bgl1-5 | TTTCCGAGGA GCGTTGCGAC GGGCTTGTTG AGTAGAGTGT GAGCTCCGTA |
| | ACTAGTTACA TCGCAAGATG TAACTGAATG AAATGGTGAA GGACGGGTCC |
| | AGCCCGCCAG GCTCGGAAAT CTTCACCAGG CGGTGGCCCG (SEQ ID NO: 47) |
| Bth1-4 | GTTTCCGAGG AGCGTTGCGA CGGGTTGTTG AGTAGAGTGT GAGCTCCGTA |
| | ACTAGTTACA TCGCAAGATG TAACTGAATG AAATGGTGAA GGACGGGTCC |
| | ACCCGCCAGG CTCGGAAATG GTCAACCGAG CGGTGTGTCT (SEQ ID NO: 48) |
| Bth1-5 | GTTTCCGAGG AGCGTTGCGA CGGGCTTGTT GAGTAGAGTG TGAGCTCCGT |
| | AACTAGTTAC ATCGCAAGAT GTAACTGAAT GAAATGGTGA AGGACGGGTC |
| | CAGCCCGCCA GGCTCGGAAA TGGTCAACCG AGCGGTGTGT CT (SEQ ID NO: 49) |
| Cac1-4 | CAATCCGAGG AGCGCTGCAG GGCCTTGTTG AGTAGAGTGT GAGCTCCGTA |
| | ACTAGTTACA TCGCAAGATG TAACTGAATG AAATGGTGAA GGACGGGTCC |
| | AGGTTTCAGG CTCGGATTCC TTAAACGGCG CTCATCCGAC (SEQ ID NO: 50) |
| Cac1-5 | CAATCCGAGG AGCGCTGCAG GGCCGTTGTT GAGTAGAGTG TGAGCTCCGT |
| | AACTAGTTAC ATCGCAAGAT GTAACTGAAT GAAATGGTGA AGGACGGGTC |
| | CACGGTTTCA GGCTCGGATT CCTTAAACGG CGCTCATCCG AC (SEQ ID NO: 51) |
| Cte1-4 | CCATCCGAGG AGCGTTGCAG CGTTTTGTTG AGTAGAGTGT GAGCTCCGTA |
| | ACTAGTTACA TCGCAAGATG TAACTGAATG AAATGGTGAA GGACGGGTCC |
| | AGGCGTGAGG CTCGGATTTC ATCCCGCAAC GACGCTCGCC CACG (SEQ ID NO: |
| | 52) |
| Cte1-5 | CCATCCGAGG AGCGTTGCAG CGTTCTTGTT GAGTAGAGTG TGAGCTCCGT |
| | AACTAGTTAC ATCGCAAGAT GTAACTGAAT GAAATGGTGA AGGACGGGTC |
| | CAGGGCGTGA GGCTCGGATT TCATCCCGCA ACGACGCTCG CCCACG (SEQ ID |
| | NO: 53) |
| Cme1-4 | CACTCCGAGG AGCGTTGCAA CGGATTGTTG AGTAGAGTGT GAGCTCCGTA |
| | ACTAGTTACA TCGCAAGATG TAACTGAATG AAATGGTGAA GGACGGGTCC |
| | ATCCGCCAGG CTCGGAATGT TTCAACGGCG CTCGCAGTTC (SEQ ID NO: 54) |
| Cme1-5 | CACTCCGAGG AGCGTTGCAA CGGATTTGTT GAGTAGAGTG TGAGCTCCGT |
| | AACTAGTTAC ATCGCAAGAT GTAACTGAAT GAAATGGTGA AGGACGGGTC |
| | CATTCCGCCA GGCTCGGAAT GTTTCAACGG CGCTCGCAGT TC (SEQ ID NO: 55) |
| Dac1-4 | CTCTCCGAGG AGCGTTGCAG CGGCTTGTTG AGTAGAGTGT GAGCTCCGTA |
| | ACTAGTTACA TCGCAAGATG TAACTGAATG AAATGGTGAA GGACGGGTCC |
| | AGTCGCGAGG CTCGGAGTCA TACCTGCAAC GACGCTCGCC CACG (SEQ ID NO: |
| | 56) |
| Dac1-5 | CTCTCCGAGG AGCGTTGCAG CGGCCTTGTT GAGTAGAGTG TGAGCTCCGT |
| | AACTAGTTAC ATCGCAAGAT GTAACTGAAT GAAATGGTGA AGGACGGGTC |
| | CAGGTCGCGA GGCTCGGAGT CATACCTGCA ACGACGCTCG CCCACG (SEQ ID |
| | NO: 57) |
| Dar1-4 | TGCCGAGGAG CGCTGCGACC CTTTGTTGAG TAGAGTGTGA GCTCCGTAAC |
| | TAGTTACATC GCAAGATGTA ACTGAATGAA ATGGTGAAGG ACGGGTCCAG |
| | GGGCCAGGCT CGGCAATGAT CAACGGCGCT CGCAAACC (SEQ ID NO: 58) |
| Dar1-5 | TGCCGAGGAG CGCTGCGACC CTTTTGTTGA GTAGAGTGTG AGCTCCGTAA |
| | CTAGTTACAT CGCAAGATGT AACTGAATGA AATGGTGAAG GACGGGTCCA |
| | GGGGGCCAGG CTCGGCAATG ATCAACGGCG CTCGCAAACC (SEQ ID NO: 37) |
| Lim1-4 | CCGTTCGAGG AGCGTTGCGA CACATTGTTG AGTAGAGTGT GAGCTCCGTA |
| | ACTAGTTACA TCGCAAGATG TAACTGAATG AAATGGTGAA GGACGGGTCC |
| | AGGTGCTAGG CTCGAACAAG AAATAAACAA CGGCGCTCAC GCTTC (SEQ ID NO: |
| | 59) |
| Lim1-5 | CCGTTCGAGG AGCGTTGCGA CACACTTGTT GAGTAGAGTG TGAGCTCCGT |
| | AACTAGTTAC ATCGCAAGAT GTAACTGAAT GAAATGGTGA AGGACGGGTC |
| | CAGGGTGCTA GGCTCGAACA AGAAATAAAC AACGGCGCTC ACGCTTC (SEQ ID |
| | NO: 60) |
| Mca1-4 | GCGCCGAGGA GCGCTGCGAC GGCTTGTTGA GTAGAGTGTG AGCTCCGTAA |
| | CTAGTTACAT CGCAAGATGT AACTGAATGA AATGGTGAAG GACGGGTCCA |
| | GCCGCCAGGC TCGGCGGGGA CAATCGGTTT TCCAACGGCG CTCTGTTTAT (SEQ |
| | ID NO: 61) |
| Mca1-5 | GCGCCGAGGA GCGCTGCGAC GGCCTTGTTG AGTAGAGTGT GAGCTCCGTA |
| | ACTAGTTACA TCGCAAGATG TAACTGAATG AAATGGTGAA GGACGGGTCC |
| | AGGCCGCCAG GCTCGGCGGG GACAATCGGT TTTCCAACGG CGCTCTGTTT AT |
| | (SEQ ID NO: 62) |
| Mpe1-4 | ACTTTCGAGG AGCGTTGCAA CTCCTTGTTG AGTAGAGTGT GAGCTCCGTA |
| | ACTAGTTACA TCGCAAGATG TAACTGAATG AAATGGTGAA GGACGGGTCC |
| | AGGAGCCAGG CTCGAAAGTC ATCCCCCGAT GCAACGGCGC TCACCCGCA (SEQ |
| | ID NO: 63) |
| Mpe1-5 | ACTTTCGAGG AGCGTTGCAA CTCCCTTGTT GAGTAGAGTG TGAGCTCCGT |
| | AACTAGTTAC ATCGCAAGAT GTAACTGAAT GAAATGGTGA AGGACGGGTC |
| | CAGGGAGCCA GGCTCGAAAG TCATCCCCCG ATGCAACGGC GCTCACCCGC A |
| | (SEQ ID NO: 39) |
| Mxa1-4 | GACTTCGAGG AGCGCTGCGA GGCCTTGTTG AGTAGAGTGT GAGCTCCGTA |
| | ACTAGTTACA TCGCAAGATG TAACTGAATG AAATGGTGAA GGACGGGTCC |
| | AGGCCCCAGG CTCGAAGCGG AATCACCCAA CGGCGCTCAC CTGTA (SEQ ID NO: |
| | 64) |
| Mxa1-5 | GACTTCGAGG AGCGCTGCGA GGCCGTTGTT GAGTAGAGTG TGAGCTCCGT |
| | AACTAGTTAC ATCGCAAGAT GTAACTGAAT GAAATGGTGA AGGACGGGTC |
| | CACGGCCCCA GGCTCGAAGC GGAATCACCC AACGGCGCTC ACCTGTA (SEQ ID |
| | NO: 42) |
| Nmo1-4 | CTCGCCGAGG AGCGCTGCAA CAGGTTGTTG AGTAGAGTGT GAGCTCCGTA |
| | ACTAGTTACA TCGCAAGATG TAACTGAATG AAATGGTGAA GGACGGGTCC |
| | ACCTGCCAGG CTCGGCGATT ATCGGGACCT TTAAACCAAC GGCGCTCGGT |
| | TCAG (SEQ ID NO: 65) |
| Nmo1-5 | CTCGCCGAGG AGCGCTGCAA CAGGATTGTT GAGTAGAGTG TGAGCTCCGT |
| | AACTAGTTAC ATCGCAAGAT GTAACTGAAT GAAATGGTGA AGGACGGGTC |
| | CATCCTGCCA GGCTCGGCGA TTATCGGGAC CTTTAAACCA ACGGCGCTCG |
| | GTTCAG (SEQ ID NO: 33) |
| Neu1-4 | TTGTACCGAG GAGCGCTGCA ACGGTTTGTT GAGTAGAGTG TGAGCTCCGT |
| | AACTAGTTAC ATCGCAAGAT GTAACTGAAT GAAATGGTGA AGGACGGGTC |
| | CACTCGTGAG GCTCGGACAA ATGAACAACC CAACAACGGC GCTCTTTCAC T |
| | (SEQ ID NO: 66) |
| Neu1-5 | TTGTACCGAG GAGCGCTGCA ACGGTCTTGT TGAGTAGAGT GTGAGCTCCG |
| | TAACTAGTTA CATCGCAAGA TGTAACTGAA TGAAATGGTG AAGGACGGGT |
| | CCATCTCGTG AGGCTCGGAC AAATGAACAA CCCAACAACG GCGCTCTTTC ACT |
| | (SEQ ID NO: 67) |
| Nmu1-4 | CAGACCGAGG AGCGCTGCAA CGGGTTGTTG AGTAGAGTGT GAGCTCCGTA |
| | ACTAGTTACA TCGCAAGATG TAACTGAATG AAATGGTGAA GGACGGGTCC |
| | ACCCGCGAGG CTCGGCAAGG AAGGATCGCT ACAACGGCGC TCGTTCATT (SEQ |
| | ID NO: 68) |
| Nmu1-5 | CAGACCGAGG AGCGCTGCAA CGGGCTTGTT GAGTAGAGTG TGAGCTCCGT |
| | AACTAGTTAC ATCGCAAGAT GTAACTGAAT GAAATGGTGA AGGACGGGTC |
| | CATCCCGCGA GGCTCGGCAA GGAAGGATCG CTACAACGGC GCTCGTTCAT T |
| | (SEQ ID NO: 36) |
| Pme1-4 | CCGTCCGAGG GGCGCTGCAG CAGGTTGTTG AGTAGAGTGT GAGCTCCGTA |
| | ACTAGTTACA TCGCAAGATG TAACTGAATG AAATGGTGAA GGACGGGTCC |
| | ATTTGTCAGG CTCGGATGGG GCGTTAACCA TACGCATCAA CGGCGCCCAT |
| | TCGCA (SEQ ID NO: 69) |
| Pme1-5 | CCGTCCGAGG GGCGCTGCAG CAGGTTTGTT GAGTAGAGTG TGAGCTCCGT |
| | AACTAGTTAC ATCGCAAGAT GTAACTGAAT GAAATGGTGA AGGACGGGTC |
| | CAGTTTGTCA GGCTCGGATG GGGCGTTAAC CATACGCATC AACGGCGCCC |
| | ATTCGCA (SEQ ID NO: 32) |
| Pol1-4 | CTTCCTGAGG AGCGTTGCAA CCCATTGTTG AGTAGAGTGT GAGCTCCGTA |
| | ACTAGTTACA TCGCAAGATG TAACTGAATG AAATGGTGAA GGACGGGTCC |
| | ATGGACCAGG CTCAGGACTT CATTGCAACC ACGCTCACCC GCA (SEQ ID NO: 70) |
| Pol1-5 | CTTCCTGAGG AGCGTTGCAA CCCACTTGTT GAGTAGAGTG TGAGCTCCGT |
| | AACTAGTTAC ATCGCAAGAT GTAACTGAAT GAAATGGTGA AGGACGGGTC |
| | CAGTGGACCA GGCTCAGGAC TTCATTGCAA CCACGCTCAC CCGCA (SEQ ID NO: |
| | 43) |
| Pae1-4 | CCGTTCGAGG GGCGCTGCAG CAGGTTGTTG AGTAGAGTGT GAGCTCCGTA |
| | ACTAGTTACA TCGCAAGATG TAACTGAATG AAATGGTGAA GGACGGGTCC |
| | ACCTGTCAGG CTCGAACGGA GCGCGCTCTC AGGCCGGCCT CGCCGGTCTG |
| | TCAACGCACC AACGGCGCCC ATTC (SEQ ID NO: 71) |
| Pae1-5 | CCGTTCGAGG GGCGCTGCAG CAGGCTTGTT GAGTAGAGTG TGAGCTCCGT |
| | AACTAGTTAC ATCGCAAGAT GTAACTGAAT GAAATGGTGA AGGACGGGTC |
| | CAGCCTGTCA GGCTCGAACG GAGCGCGCTC TCAGGCCGGC CTCGCCGGTC |
| | TGTCAACGCA CCAACGGCGC CCATTC (SEQ ID NO: 72) |
| Pfl2-4 | CTGTCCGAGG GGCGCTGCAG CAGGTTGTTG AGTAGAGTGT GAGCTCCGTA |
| | ACTAGTTACA TCGCAAGATG TAACTGAATG AAATGGTGAA GGACGGGTCC |
| | ACCTGTCAGG CTCGGATGGG GCGTTGTTGG TACAGGGCTC CCTGTATGGA |
| | CACTAAACGC ACAACGGCGC CCAT (SEQ ID NO: 73) |
| Pfl2-5 | CTGTCCGAGG GGCGCTGCAG CAGGCTTGTT GAGTAGAGTG TGAGCTCCGT |
| | AACTAGTTAC ATCGCAAGAT GTAACTGAAT GAAATGGTGA AGGACGGGTC |
| | CAACCTGTCA GGCTCGGATG GGGCGTTGTT GGTACAGGGC TCCCTGTATG |
| | GACACTAAAC GCACAACGGC GCCCAT (SEQ ID NO: 74) |
| Ppu1-4 | CTGTCCGAGG GGCGCTGCAG CAGGTTGTTG AGTAGAGTGT GAGCTCCGTA |
| | ACTAGTTACA TCGCAAGATG TAACTGAATG AAATGGTGAA GGACGGGTCC |
| | ACCTGTCAGG CTCGGATGGG GCGTTGCTCG CTCCCGCGAG CGCTTAACGC |
| | ACAACGGCGC CCATTCGCA (SEQ ID NO: 75) |
| Ppu1-5 | CTGTCCGAGG GGCGCTGCAG CAGGCTTGTT GAGTAGAGTG TGAGCTCCGT |
| | AACTAGTTAC ATCGCAAGAT GTAACTGAAT GAAATGGTGA AGGACGGGTC |
| | CAGCCTGTCA GGCTCGGATG GGGCGTTGCT CGCTCCCGCG AGCGCTTAAC |
| | GCACAACGGC GCCCATTCGC A (SEQ ID NO: 76) |
| Pst1-4 | CCACCCGAGG GGCGCTGCAG CAGGTTGTTG AGTAGAGTGT GAGCTCCGTA |
| | ACTAGTTACA TCGCAAGATG TAACTGAATG AAATGGTGAA GGACGGGTCC |
| | ACCTGTCAGG CTCGGATGGG GCGTTTCCTC GAACGCATCA ACGGCGCCCA |
| | TTCGCA (SEQ ID NO: 77) |
| Pst1-5 | CCACCCGAGG GGCGCTGCAG CAGGTTTGTT GAGTAGAGTG TGAGCTCCGT |
| | AACTAGTTAC ATCGCAAGAT GTAACTGAAT GAAATGGTGA AGGACGGGTC |
| | CAACCTGTCA GGCTCGGATG GGGCGTTTCC TCGAACGCAT CAACGGCGCC |
| | CATTCGCA (SEQ ID NO: 78) |
| Psy1-4 | CCTGTCCGAG GGGCGCTGCA GCAGTTGTTG AGTAGAGTGT GAGCTCCGTA |
| | ACTAGTTACA TCGCAAGATG TAACTGAATG AAATGGTGAA GGACGGGTCC |
| | ACTGTCAGGC TCGGATGGGG CGTTGTTTGT CAGGTTGATC ACAGCCGGCA |
| | AGCCTTAAAC GCACAACGGC GCCCATTCGC A (SEQ ID NO: 79) |
| Psy1-5 | CCTGTCCGAG GGGCGCTGCA GCAGGTTGTT GAGTAGAGTG TGAGCTCCGT |
| | AACTAGTTAC ATCGCAAGAT GTAACTGAAT GAAATGGTGA AGGACGGGTC |
| | CACCTGTCAG GCTCGGATGG GGCGTTGTTT GTCAGGTTGA TCACAGCCGG |
| | CAAGCCTTAA ACGCACAACG GCGCCCATTC GCA (SEQ ID NO: 80) |
| Rfea1-4 | TTCCGAGGAG CGTTGCAGCT CTTGTTGAGT AGAGTGTGAG CTCCGTAACT |
| | AGTTACATCG CAAGATGTAA CTGAATGAAA TGGTGAAGGA CGGGTCCAGA |
| | GTGAGGCTCG GAGCACGCGG CCCTGATGGG GCTGCGACAG TCAACGGCGC |
| | TCACCCACT (SEQ ID NO: 81) |
| Rfea1-5 | TTCCGAGGAG CGTTGCAGCT CGTTGTTGAG TAGAGTGTGA GCTCCGTAAC |
| | TAGTTACATC GCAAGATGTA ACTGAATGAA ATGGTGAAGG ACGGGTCCAC |
| | GAGTGAGGCT CGGAGCACGC GGCCCTGATG GGGCTGCGAC AGTCAACGGC |
| | GCTCACCCAC T (SEQ ID NO: 82) |
| Rfem1-4 | TCTTCCAAGG AGCGTTGCAG TCGGTTGTTG AGTAGAGTGT GAGCTCCGTA |
| | ACTAGTTACA TCGCAAGATG TAACTGAATG AAATGGTGAA GGACGGGTCC |
| | ACCGGTCAGG CTTGGATGAC CCCAACGACG CTCACCTGAT (SEQ ID NO: 83) |
| Rfem1-5 | TCTTCCAAGG AGCGTTGCAG TCGGCTTGTT GAGTAGAGTG TGAGCTCCGT |
| | AACTAGTTAC ATCGCAAGAT GTAACTGAAT GAAATGGTGA AGGACGGGTC |
| | CAGCCGGTCA GGCTTGGATG ACCCCAACGA CGCTCACCTG AT (SEQ ID NO: 38) |
| Rso1-4 | TTCCGAGGAG CGTTGCAACG GCTTGTTGAG TAGAGTGTGA GCTCCGTAAC |
| | TAGTTACATC GCAAGATGTA ACTGAATGAA ATGGTGAAGG ACGGGTCCAG |
| | CCGCCAGGCT CGGAAGTTCA AACGGCGCTC GCACTAT (SEQ ID NO: 84) |
| Rso1-5 | TTCCGAGGAG CGTTGCAACG GCCTTGTTGA GTAGAGTGTG AGCTCCGTAA |
| | CTAGTTACAT CGCAAGATGT AACTGAATGA AATGGTGAAG GACGGGTCCA |
| | GGCCGCCAGG CTCGGAAGTT CAAACGGCGC TCGCACTAT (SEQ ID NO: 85) |
| Sma1-4 | CTGCCGAGGG GCGCTGCGAC CGGTTGTTGA GTAGAGTGTG AGCTCCGTAA |
| | CTAGTTACAT CGCAAGATGT AACTGAATGA AATGGTGAAG GACGGGTCCA |
| | CCGGCCAGGC TCGGCCAGGT GGTACTGCAA CAACGGCGCC CGGCCAGA (SEQ |
| | ID NO: 86) |
| Sma1-5 | CTGCCGAGGG GCGCTGCGAC CGGATTGTTG AGTAGAGTGT GAGCTCCGTA |
| | ACTAGTTACA TCGCAAGATG TAACTGAATG AAATGGTGAA GGACGGGTCC |
| | AGCCGGCCAG GCTCGGCCAG GTGGTACTGC AACAACGGCG CCCGGCCAGA |
| | (SEQ ID NO: 87) |
| Sau1-4 | CAACCTGAGG AGCGCTGCAA CGGGTTGTTG AGTAGAGTGT GAGCTCCGTA |
| | ACTAGTTACA TCGCAAGATG TAACTGAATG AAATGGTGAA GGACGGGTCC |
| | ACCCGCCAGG CTCAGGTTCA TGTAACGGCG CTCGCTGACT (SEQ ID NO: 88) |
| Sau1-5 | CAACCTGAGG AGCGCTGCAA CGGGGTTGTT GAGTAGAGTG TGAGCTCCGT |
| | AACTAGTTAC ATCGCAAGAT GTAACTGAAT GAAATGGTGA AGGACGGGTC |
| | CATCCCGCCA GGCTCAGGTT CATGTAACGG CGCTCGCTGA CT (SEQ ID NO: 89) |
| Veil-4 | CGCTCCGAGG AGCGTTGCAG CGCCTTGTTG AGTAGAGTGT GAGCTCCGTA |
| | ACTAGTTACA TCGCAAGATG TAACTGAATG AAATGGTGAA GGACGGGTCC |
| | AGGCGTGAGG CTCGGGCATC ATCTGGCAAC GACGCTCATC CACA (SEQ ID NO: |
| | 90) |
| Veil-5 | CGCTCCGAGG AGCGTTGCAG CGCCTTTGTT GAGTAGAGTG TGAGCTCCGT |
| | AACTAGTTAC ATCGCAAGAT GTAACTGAAT GAAATGGTGA AGGACGGGTC |
| | CAGGGCGTGA GGCTCGGGCA TCATCTGGCA ACGACGCTCA TCCACA (SEQ ID |
| | NO: 91) |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

Notwithstanding the appended claims, the disclosure set forth herein is also described by the following clauses.

1. A single stranded nucleic acid, comprising:

a S-adenosylhomocysteine (SAH)-binding riboswitch domain comprising:
a 5′-terminal domain comprising the following sequence: YYRAGGRGCGYUGCRR (SEQ ID NO:102), wherein Y is C or U and R is G or A;
a 3′-terminal domain comprising the following sequences: YCAGGCUYRR (SEQ ID NO:103) and CAACGRCGCYCR (SEQ ID NO: 104), wherein Y is C or U and R is G or A; and
a P2′ stem; and

a contiguous Spinach aptamer domain terminated at a P2 stem that is operably connected to the P2′ stem of the SAH-binding riboswitch domain via a P2/P2′ stem comprising 5 base pairs or less.

2. The nucleic acid of clause 1, wherein the 5′-terminal domain comprises a sequence having at least 80% sequence identity to the following sequence: CCGAGGGGCGCUGCAG (SEQ ID NO: 105).

3. The nucleic acid of clause 1, wherein the 5′-terminal domain comprises the following sequence: CCGAGGGGCGCUGCAG (SEQ ID NO: 105).

4. The nucleic acid of any one of clauses 1-3, wherein the 3′-terminal domain comprises a sequence having at least 80% sequence identity to the following sequence: UCAGGCUCGG (SEQ ID NO: 106).

5. The nucleic acid of any one of clauses 1-4, wherein the 3′-terminal domain comprises a sequence having at least 80% sequence identity to the following sequence: CAACGGCGCCCA (SEQ ID NO: 107).

6. The nucleic acid of any one of clauses 1-3, wherein the 3′-terminal domain comprises the following sequences: UCAGGCUCGG (SEQ ID NO: 106) and CAACGGCGCCCA (SEQ ID NO: 107).

7. A nucleic acid construct encoding the single stranded nucleic acid of any one of clauses 1-6.

8. A host cell comprising the nucleic acid construct of clause 7.

9. A biosensor, comprising:

a single stranded nucleic acid comprising:
a S-adenosylhomocysteine (SAH)-binding riboswitch domain comprising a P2′ stem; and
a contiguous Spinach aptamer domain terminated at a P2 stem that is operably connected to the P2′ stem of the SAH-binding riboswitch domain via a P2/P2′ stem of 5 base pairs or less in length; and

a signaling chromophore specifically bound to the Spinach aptamer domain;

wherein the sensor is configured to fluorescently activate the signaling chromophore upon specific binding of SAH to the SAH-binding riboswitch domain.

10. The biosensor of clause 9, wherein the fluorescence activation of the signaling chromophore is by 40% or more.

11. The biosensor of clause 9 or 10, wherein the biosensor is configured to specifically bind SAH with at least 10-fold stronger affinity over SAM.

12. The biosensor of any one of clauses 9-11, comprising the single stranded nucleic acid of any one of clauses 1-6.

13. A method for determining the level of SAH in a sample, the method comprising:

contacting the sample with a biosensor according to any one of clauses 9-12; and

detecting fluorescence from the biosensor thereby determining the level of SAH in the sample.

14. The method of clause 13, wherein the determined level of SAH in the sample is independent of the level of SAM in the sample.

15. The method of clause 13 or 14, further comprising determining a methyltransferase activity of the sample based on the determined level of SAH.

16. The method of clause 15, wherein the sample is a cellular sample.

17. A method for determining level of methyltransferase activity in a cell, the method comprising:

contacting the cell with a single stranded nucleic acid according to any one of clauses 1-6 and a signaling chromophore to produce a SAH biosensor in situ; and

detecting fluorescence from the signaling chromophore of the SAH biosensor thereby determining the level of methyltransferase activity in the cell.

18. The method of clause 17, wherein the single stranded nucleic acid is expressed by the cell.

19. The method of clause 17 or 18, further comprising monitoring fluorescence of the signaling chromophore upon application of a stimulus to the cell.

20. A kit comprising:

a single stranded nucleic acid of any one of clauses 1-6 or a nucleic acid construct encoding the single stranded nucleic acid of any one of clauses 1-6; and

one or more components selected from a signaling chromophore, SAH, SAM, a promoter, a cell, a cloning vector and an expression cassette.

What is claimed is:

1. A single stranded nucleic acid, comprising:
a S-adenosylhomocysteine (SAH)-binding riboswitch domain comprising:
a 5′-terminal domain comprising the following sequence: YYRAGGRGCGYUGCRR (SEQ ID NO:102), wherein Y is C or U and R is G or A;
a 3′-terminal domain comprising the following sequences:
YCAGGCUYRR (SEQ ID NO:103) and CAACGRCGCYCR (SEQ ID NO: 104), wherein Y is C or U and R is G or A; and
a P2′ stem; and
a contiguous Spinach aptamer domain terminated at a P2 stem that is operably connected to the P2′ stem of the SAH-binding riboswitch domain via a P2/P2′ stem comprising 5 base pairs or less.

2. The nucleic acid of claim 1, wherein the 5′-terminal domain comprises a sequence having at least 80% sequence identity to the following sequence: CCGAGGGGCGCUGCAG (SEQ ID NO: 105).

3. The nucleic acid of claim 1, wherein the 5′-terminal domain comprises the following sequence: CCGAGGGGCGCUGCAG (SEQ ID NO: 105).

4. The nucleic acid of claim 1, wherein the 3′-terminal domain comprises a sequence having at least 80% sequence identity to the following sequence: UCAGGCUCGG (SEQ ID NO: 106).

5. The nucleic acid of claim 1, wherein the 3′-terminal domain comprises a sequence having at least 80% sequence identity to the following sequence: CAACGGCGCCCA (SEQ ID NO: 107).

6. The nucleic acid of claim 1, wherein the 3′-terminal domain comprises the following sequences: UCAGGCUCGG (SEQ ID NO: 106) and CAACGGCGCCCA (SEQ ID NO: 107).

7. A nucleic acid construct encoding the single stranded nucleic acid of claim 1.

8. A host cell comprising the nucleic acid construct of claim 7.

9. A biosensor, comprising:
a single stranded nucleic acid comprising:
a S-adenosylhomocysteine (SAH)-binding riboswitch domain comprising a P2′ stem; and
a contiguous Spinach aptamer domain terminated at a P2 stem that is operably connected to the P2′ stem of the SAH-binding riboswitch domain via a P2/P2′ stem of 5 base pairs or less in length; and
a signaling chromophore specifically bound to the Spinach aptamer domain;
wherein the sensor is configured to fluorescently activate the signaling chromophore upon specific binding of SAH to the SAH-binding riboswitch domain.

10. The biosensor of claim 9, wherein the fluorescence activation of the signaling chromophore is by 40% or more.

11. The biosensor of claim 9, wherein the biosensor is configured to specifically bind SAH with at least 10-fold stronger affinity over SAM.

12. The biosensor of claim 9, comprising the single stranded nucleic acid of claim 1.

13. A method for determining the level of SAH in a sample, the method comprising:
contacting the sample with a biosensor according to claim 9; and
detecting fluorescence from the biosensor thereby determining the level of SAH in the sample.

14. The method of claim 13, wherein the determined level of SAH in the sample is independent of the level of SAM in the sample.

15. The method of claim 13, further comprising determining a methyltransferase activity of the sample based on the determined level of SAH.

16. The method of claim 15, wherein the sample is a cellular sample.

17. A method for determining level of methyltransferase activity in a cell, the method comprising:
contacting the cell with a single stranded nucleic acid according to claim 1 and a signaling chromophore to produce a SAH biosensor in situ; and
detecting fluorescence from the signaling chromophore of the SAH biosensor thereby determining the level of methyltransferase activity in the cell.

18. The method of claim 17, wherein the single stranded nucleic acid is expressed by the cell.

19. The method of claim 17, further comprising monitoring fluorescence of the signaling chromophore upon application of a stimulus to the cell.

20. A kit comprising:
a single stranded nucleic acid of claim 1 or a nucleic acid construct encoding the single stranded nucleic acid of claim 1; and
one or more components selected from a signaling chromophore, SAH, SAM, a promoter, a cell, a cloning vector and an expression cassette.
